(19) United States
(12) Patent Application Publication
(10) Pub. No.: US 2018/0251548 A1
(43) Pub. Date: Sep. 6, 2018

(54) COMPOSITIONS AND METHODS FOR TREATING CANCER VIA ANTAGONISM OF THE CD155/TIGIT PATHWAY AND TGF-BETA

(71) Applicant: Compass Therapeutics LLC, Cambridge, MA (US)

(72) Inventors: Helen SABZEVARI, Cambridge, MA (US); Simon METENOU, Cambridge, MA (US)

(21) Appl. No.: 15/758,090

(22) PCT Filed: Sep. 14, 2016
(86) PCT No.: PCT/US2016/051704

§ 371 Date: Mar. 7, 2018

Related U.S. Application Data
(60) Provisional application No. 62/218,227, filed on Sep. 14, 2015.
(60) Provisional application No. 62/218,169, filed on Sep. 14, 2015.

(51) Int. Cl. C07K 16/28; C07K 16/30; A61P 35/00

(52) U.S. Cl. CPC C07K 16/2803; C07K 16/2863; C07K 16/2896; A61K 38/1774; A61P 35/00; A61K 2300/00; C07K 16/3015

(57) ABSTRACT

The present disclosure relates the combined effect of anti-CD155 [CD155, Nectin-like protein 5 (Nec1-5), Tage4, HVED, poliovirus receptor (PVR)] antibodies or anti TIGIT antibodies [TIGIT (T cell immunoreceptor with Ig and ITIM domains), WUCAM (Washington University cell adhesion molecule), Vstm3 (V-set and transmembrane domaincontaining protein 3), Vsig9 (V-set and immunoglobulin domain-containing protein 9)] with a TGF Beta 1 antagonist. The effect is measured by increased IFN-gamma production human breast adenocarcinoma cells. Use of combined antibodies /CD155 or TIGIT) with a TGF-Beta 1 antagon-ist to treat a patient with cancer is claimed.

### RELATED APPLICATIONS

This application claims the benefit of the priority date of U.S. Provisional Application No. 62/218,227 which was filed Sep. 14, 2015; and U.S. Provisional Application No. 62/218,169, which was filed Sep. 14, 2015. The content of each provisional application is hereby incorporated by reference in its entirety.

### BACKGROUND

Tumors constitute highly suppressive microenvironments in which the function of infiltrating immune cells, such as T cells, is downregulated by various molecular mechanisms.

Antibody blockade of T cell co-inhibitory molecules, also known as immune checkpoints, has recently emerged as a treatment for cancer. Antibodies targeting CTLA-4, PD-1, and PD-L1 have shown therapeutic benefit in human clinical trials. Specifically, the anti-CTLA-4 antibody ipilimumab (Yervoy®) and the anti-PD-1 antibodies nivolumab (Opdivo®) and pembrolizumab (Keytruda®), are currently approved for treating cancer patients.

However, there remains a need for additional and improved compositions and methods to modulate the activity of immunoinhibitory proteins. Such agents can be used for cancer immunotherapy and treatment of other conditions, such as chronic infection.

### SUMMARY OF THE DISCLOSURE

The present disclosure is based, in part, on the discovery that CD155 is highly expressed on many types of cancer cells, and that use of a CD155/TIGIT pathway antagonist in combination with a TGF-β1 antagonist enhances the activation of T cells exposed to cancer cells, as compared to the level of T cell activation in the presence of either antagonist alone. For example, as described in the working examples (infra), Th1 cytokine production (i.e., IL-2 and IFNγ) by CD4+ and CD8+ T cells exposed to cancer cells in the presence of agents that simultaneously neutralize both the CD155/TIGIT and TGF-β pathways was significantly enhanced as compared to the level of cytokine production by such cells under conditions in which only one of the pathways was inhibited. At least because Th1 cytokine production by T cells is associated with productive anti-tumor immune responses, administration of a CD155/TIGIT pathway antagonist in combination with a TGF-β1 antagonist is useful for treating cancer.

Accordingly, in some aspects, the disclosure relates to methods of treating cancer in a patient comprising administering to the patient an effective amount of a CD155/TIGIT antagonist and a TGF-β1 antagonist, thereby treating the patient.

In other aspects, the disclosure relates to methods for treating cancer in a patient who has received or is receiving treatment with a TGF-β1 antagonist, the method comprising administering to the patient an effective amount of a CD155/TIGIT antagonist, thereby treating the patient. In other aspects, the disclosure relates to methods for treating cancer in a patient who has received or is receiving treatment with a CD155/TIGIT antagonist, the method comprising administering to the patient an effective amount of a TGF-β1 antagonist, thereby treating the patient.

In another aspect, the disclosure relates to a CD155/TIGIT antagonist and a TGF-β1 antagonist for use in treating cancer in a patient, wherein the treatment comprises administering to the patient an effective amount of a CD155/TIGIT antagonist and an effective amount of a TGF-β1 antagonist, thereby treating the patient. In other aspects, the disclosure relates to a CD155/TIGIT antagonist for use in treating cancer in a patient who has received or is receiving treatment with a TGF-β1 antagonist, wherein the treatment comprises administering to the patient an effective amount of a CD155/TIGIT antagonist, thereby treating the patient. In yet other aspects, the disclosure relates to a TGF-β1 antagonist for use in treating cancer in a patient who has received or is receiving treatment with a CD155/TIGIT antagonist, wherein the treatment comprises administering to the patient an effective amount of a TGF-β1 antagonist, thereby treating the patient.

In these and other aspects of the disclosure, the methods and uses described herein comprise a cancer-specific immune response in the patient.

Another aspect of the disclosure relates to methods for enhancing a cancer-specific immune response in a cancer patient who has received or is receiving treatment with a TGF-β1 antagonist, the method comprising administering to the patient an effective amount of a CD155/TIGIT antagonist, thereby enhancing a cancer-specific immune response in the patient as compared to the cancer-specific immune response in the patient following administration of the TGF-β1 antagonist alone. In other aspects, the disclosure relates to methods for enhancing a cancer-specific immune response in a cancer patient who has received or is receiving treatment with a CD155/TIGIT antagonist, the method comprising administering to the patient an effective amount of a TGF-β1 antagonist thereby enhancing a cancer-specific immune response in the patient as compared to the cancer-specific immune response in the patient following administration of the CD155/TIGIT antagonist alone. In some aspects, the disclosure relates to methods for enhancing a cancer-specific immune response in a cancer patient, the method comprising administering to the patient an effective amount of: (i) a CD155/TIGIT antagonist; and (ii) a TGF-β1 antagonist, thereby enhancing a cancer-specific immune response in the patient as compared to the cancer-specific immune response in the patient following administration of either the TGF-β1 antagonist or the CD155/TIGIT antagonist alone.

Another aspect of the disclosure relates to methods for treating cancer in a patient, the method comprising administering to the patient an effective amount of:

(i) a CD155/TIGIT antagonist, wherein the CD155/TIGIT antagonist is an anti-CD155 antibody or antigen-binding fragment thereof, or an anti-TIGIT antibody or antigen-binding fragment thereof; and

(ii) a TGF-β1 antagonist, wherein the TGF-β1 antagonist comprises a fusion protein comprising an extracellular domain of the type 2 TGF-β receptor or a TGF-β1-binding fragment thereof and a human IgG1 Fc domain,

thereby treating the patient.

Another aspect of the disclosure relates to methods for enhancing a cancer-specific immune response in a cancer patient, the method comprising administering to the patient an effective amount of:

(i) a CD155/TIGIT antagonist, wherein the CD155/TIGIT antagonist is an anti-CD155 antibody or antigen-binding fragment thereof, or an anti-TIGIT antibody or antigen-binding fragment thereof; and

(ii) a TGF-β1 antagonist, wherein the TGF-β1 antagonist comprises a fusion protein comprising an extracellular domain of the type 2 TGF-β receptor or a TGF-β1-binding fragment thereof and a human IgG1 Fc domain,

thereby enhancing a cancer-specific immune response in the patient as compared to the cancer-specific immune response in the patient following administration of either the TGF-β1 antagonist or the CD155/TIGIT antagonist alone.

In these and other aspects of the disclosure, cancer cells of the patient express CD155. In other aspects, the cancer cells overexpress CD155 relative to normal cells of the same histological type. In some aspects, any of the methods disclosed herein further comprise determining whether cancer cells of the patient express or overexpress CD155.

In any of the aspects disclosed herein, the CD155/TIGIT antagonist is an anti-CD155 antibody or an antigen-binding fragment thereof. In these and other aspects of the disclosure, the CD155/TIGIT antagonist is an anti-TIGIT antibody or an antigen-binding fragment thereof. In some aspects, the antibody is a humanized antibody or a fully human antibody. In some embodiments, the CD155/TIGIT antagonist is a non-antibody, scaffold protein.

In these and other aspects of the disclosure, the TGF-β1 antagonist comprises a small molecule, nucleic acid or polypeptide. In some aspects, the TGF-β1 antagonist is a soluble form of a TGF-β receptor protein. In some aspects, the TGF-β receptor protein is dimeric. In other aspects, the TGF-β receptor protein is an extracellular domain of the type 2 TGF-β receptor or a TGF-β1-binding fragment thereof. In some aspects, the TGF-β receptor protein comprises the amino acid sequence depicted in SEQ ID NO: 5.

In these and other aspects of the disclosure, the TGF-β1 antagonist comprises a fusion protein comprising an extracellular domain of the type 2 TGF-β receptor, or a TGF-β1-binding fragment thereof, and a moiety that enhances the serum half-life of the TGF-β1 antagonist. In some aspects, the moiety that enhances the serum half-life of the TGF-β1 antagonist is polyethylene glycol, an Fc portion of an antibody, or an albumin protein. In other aspects, the TGF-β1 antagonist comprises a fusion protein comprising an extracellular domain of the type 2 TGF-β receptor or a TGF-β1-binding fragment thereof and a human IgG Fc domain. In some aspects, the human IgG Fc domain is a human IgG1 Fc domain, a human IgG2 Fc domain, a human IgG3 Fc domain, or a human IgG4 Fc domain. In further aspects, the TGF-β1 antagonist comprises (a) an extracellular domain of the type 2 TGF-β receptor comprising the amino acid sequence depicted in SEQ ID NO: 5, and (b) a human IgG1 Fc domain comprising the amino acid sequence depicted in SEQ ID NO: 2.

In these and other aspects of the disclosure, the cancer-specific immune response is a T cell response. In some aspects, the T cell response in the patient is greater than the T cell response following administration of either the CD155/TIGIT antagonist or the TGF-β1 antagonist alone. In other aspects, the T cell response comprises the production of IFNγ by one or both of CD4⁺ T cells and CD8⁺ T cells. In yet further aspects, the T cell response comprises the production of IL-2 by one or both of CD4⁺ T cells and CD8⁺ T cells. In other aspects, the T cell response comprises proliferation of one or both of CD4⁺ T cells and CD8⁺ T cells.

In further aspects, the disclosure provides methods comprising delaying cancer progression in the patient.

In some aspects, the disclosure provides methods comprising enhancing a tumor-specific immune response in the patient.

### BRIEF DESCRIPTION OF THE FIGURES

FIG. 1 is a schematic of the proposed model of enhancement of tumor immunity via CD155 pathway blockade with an anti-CD155 antibody.

FIGS. 2A-2C are bar graphs showing the frequency of mutations, deletions, amplifications or multiple alterations amongst TIGIT (FIG. 2A), CD155 (FIG. 2B) and TGF-β (FIG. 2C) genes in various cancers.

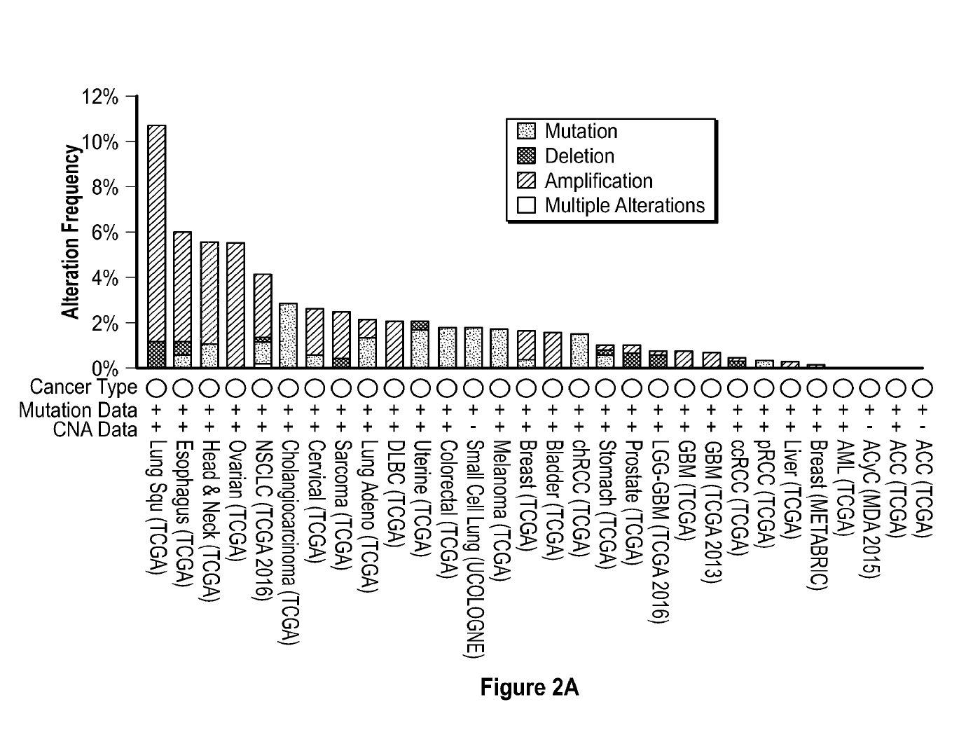

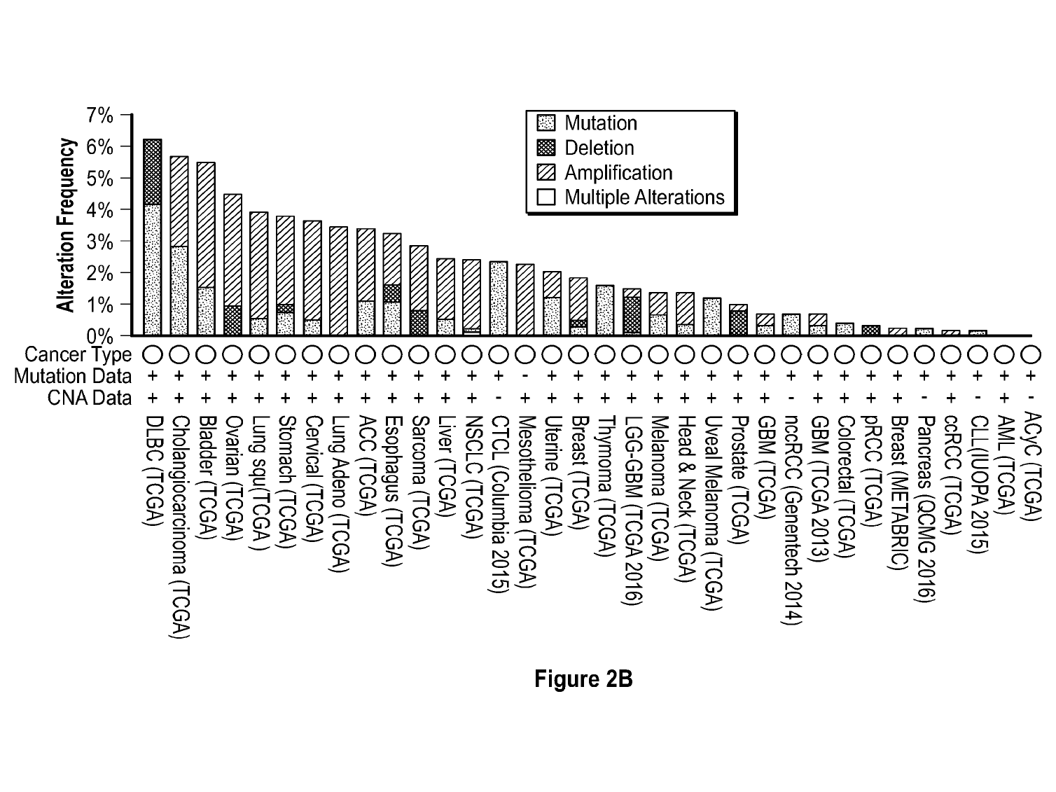

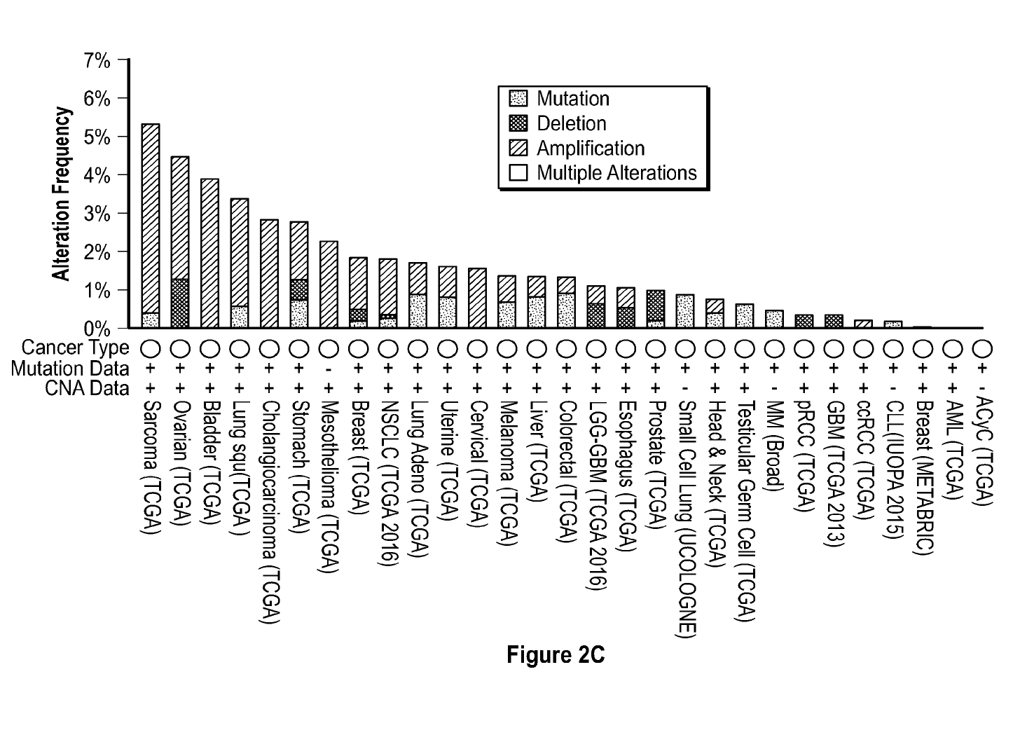

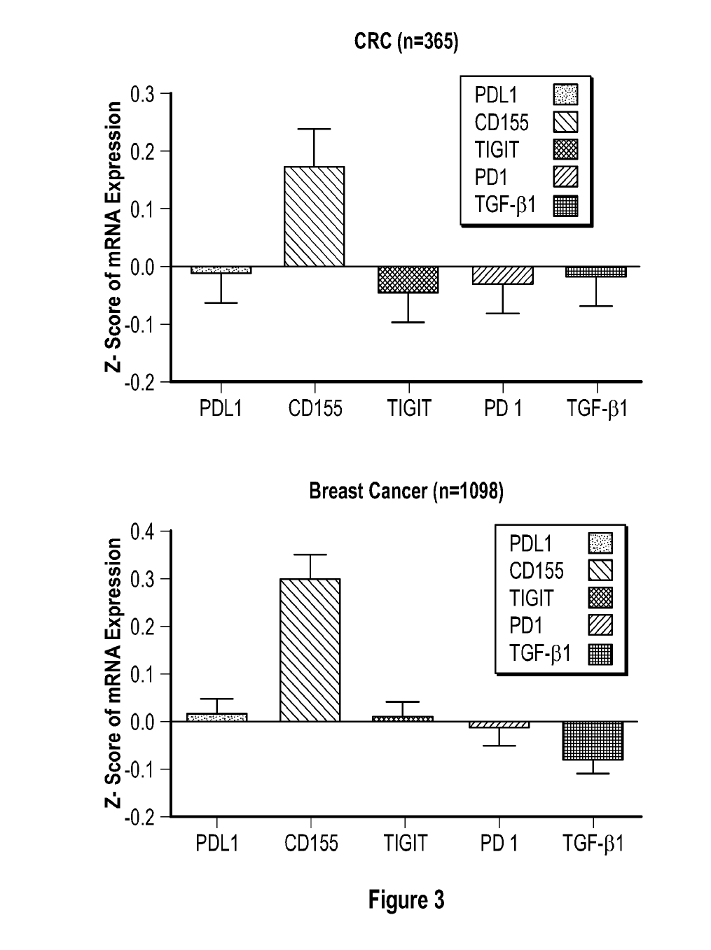

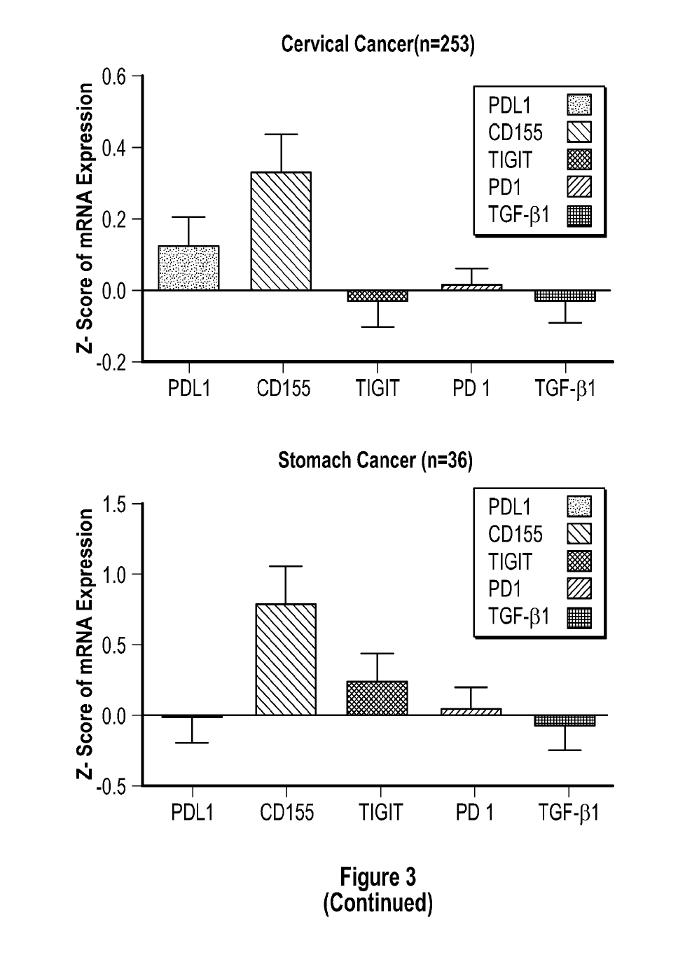

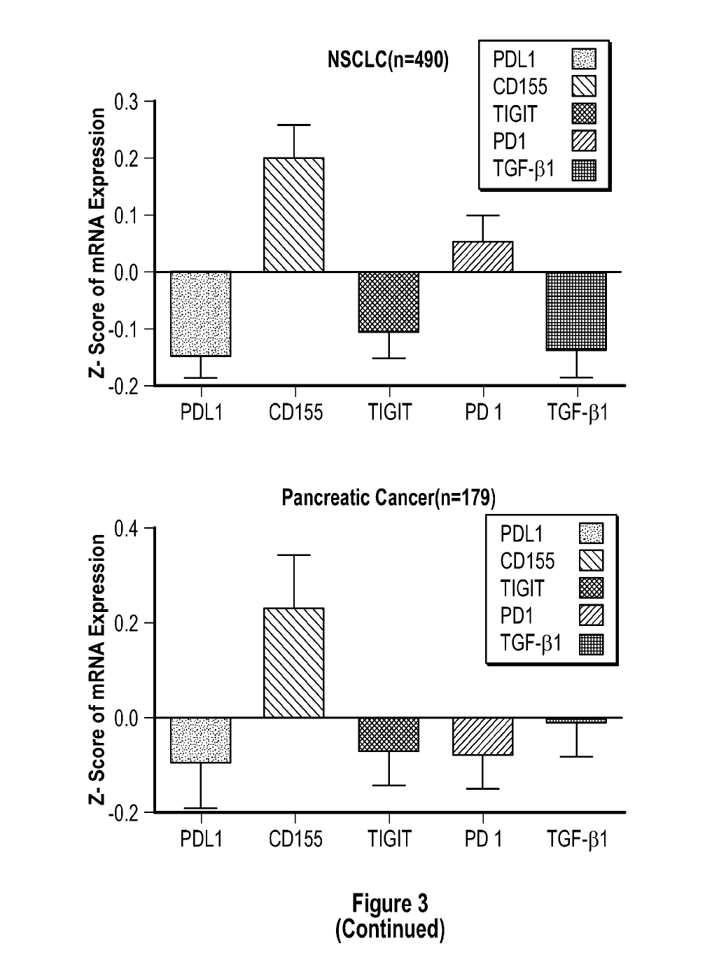

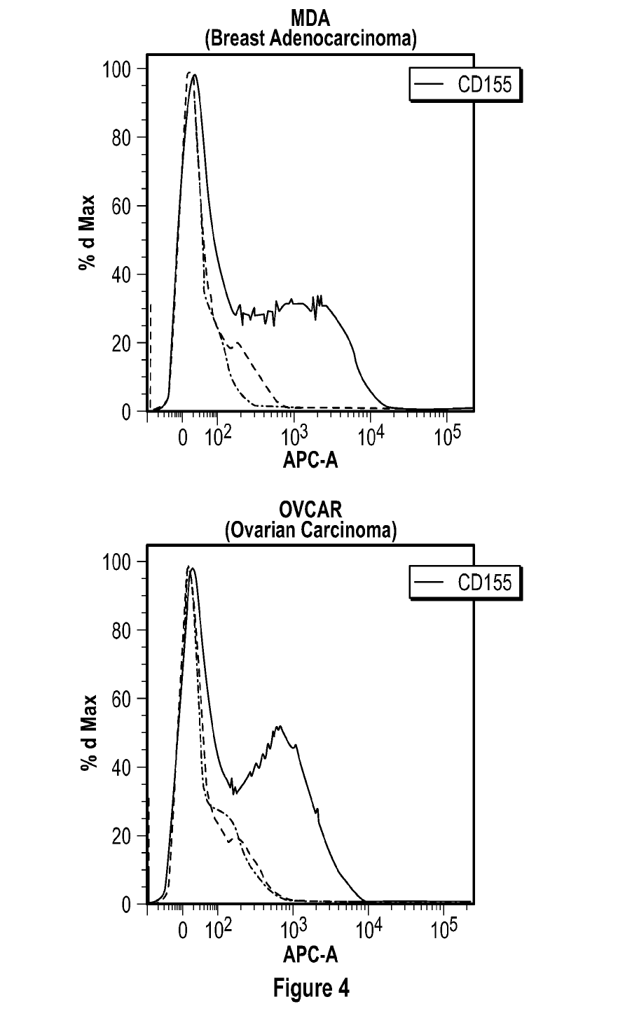

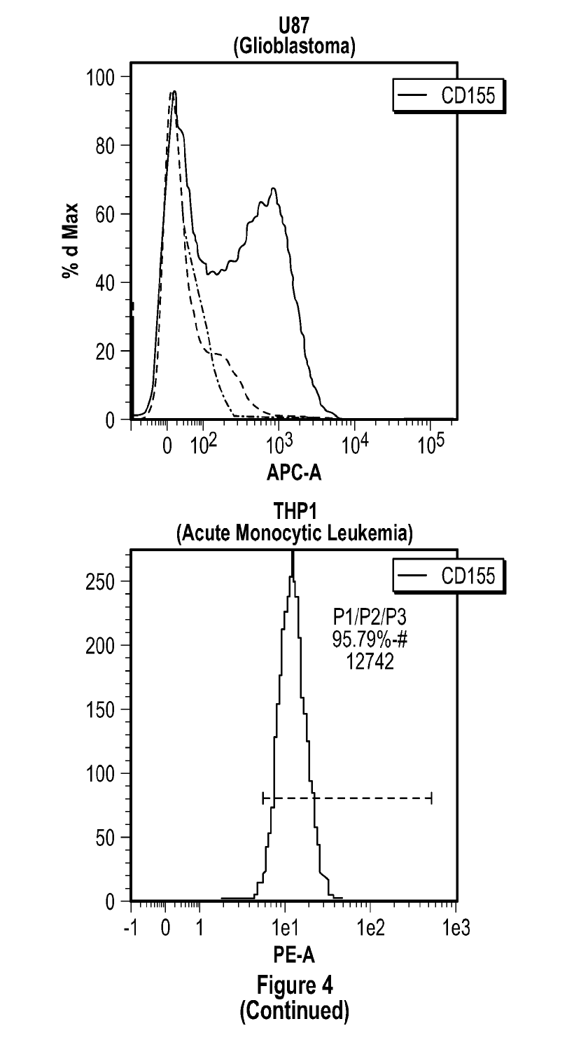

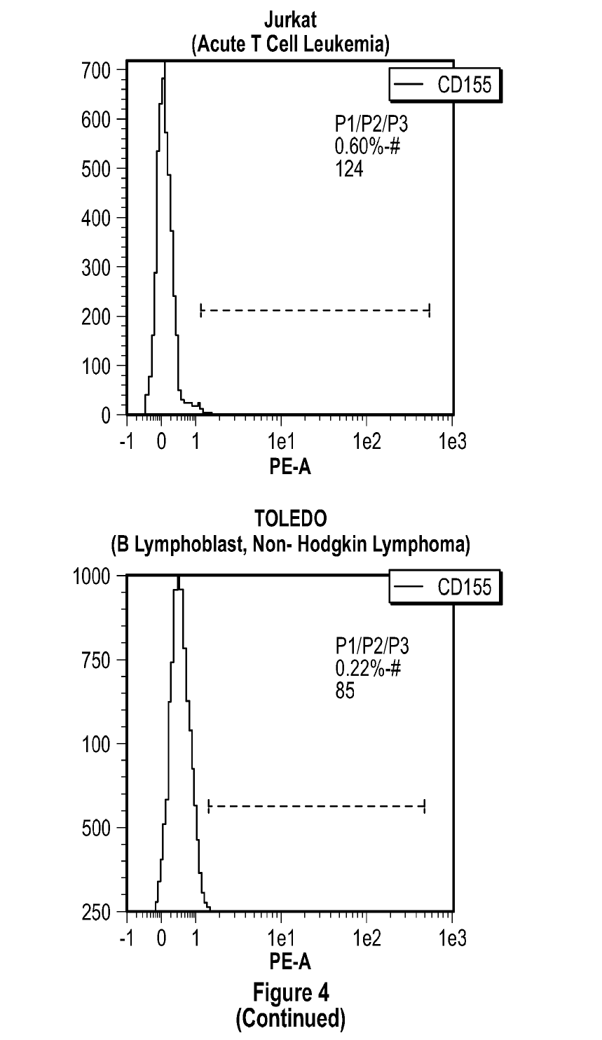

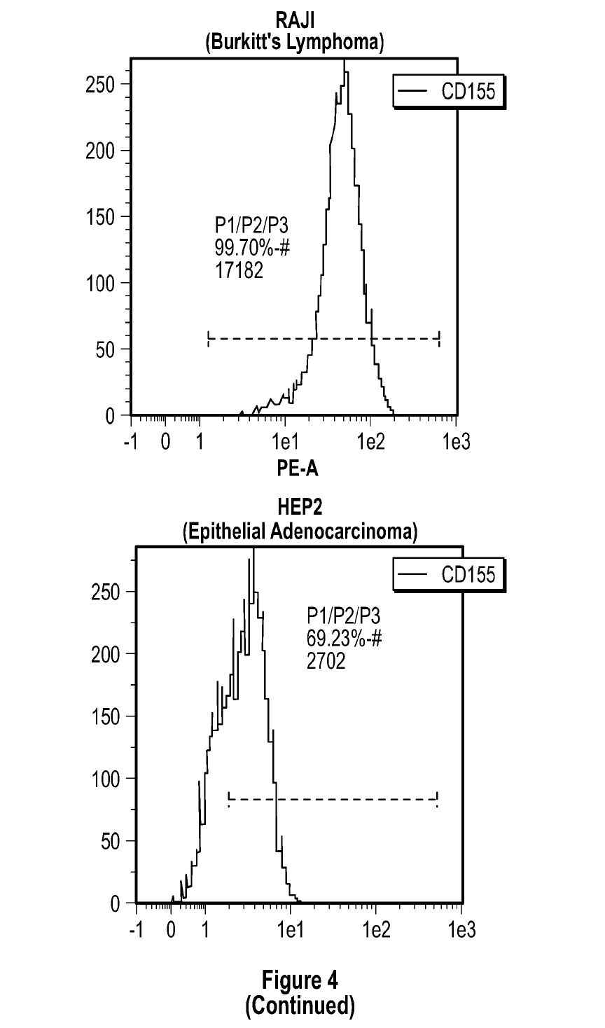

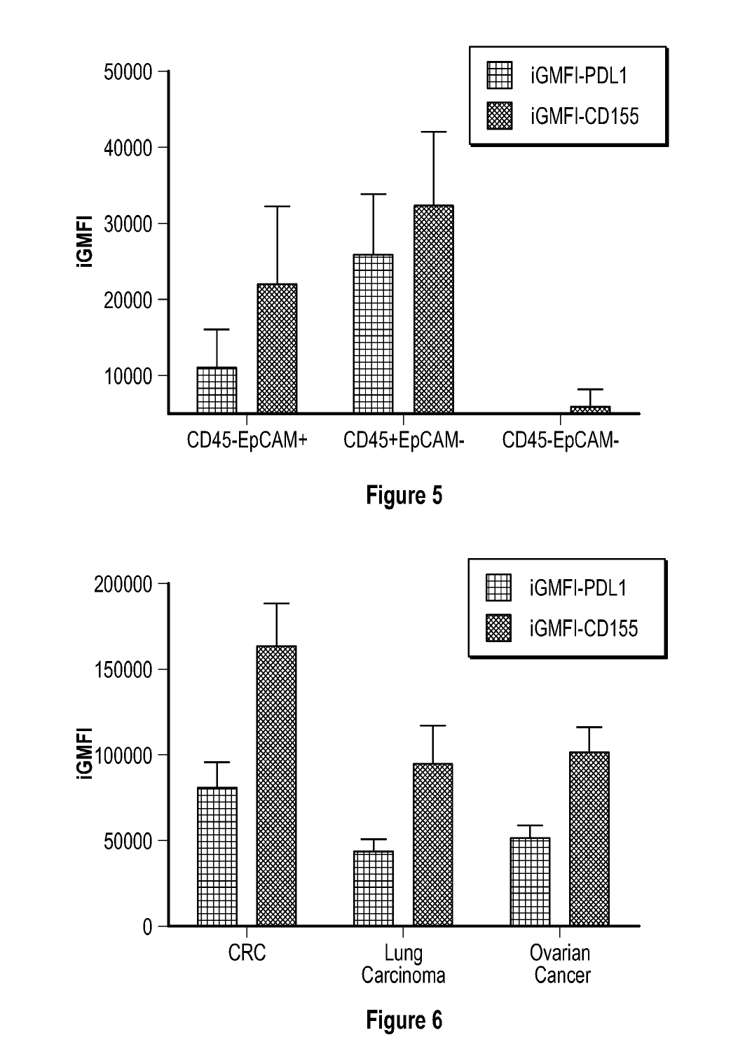

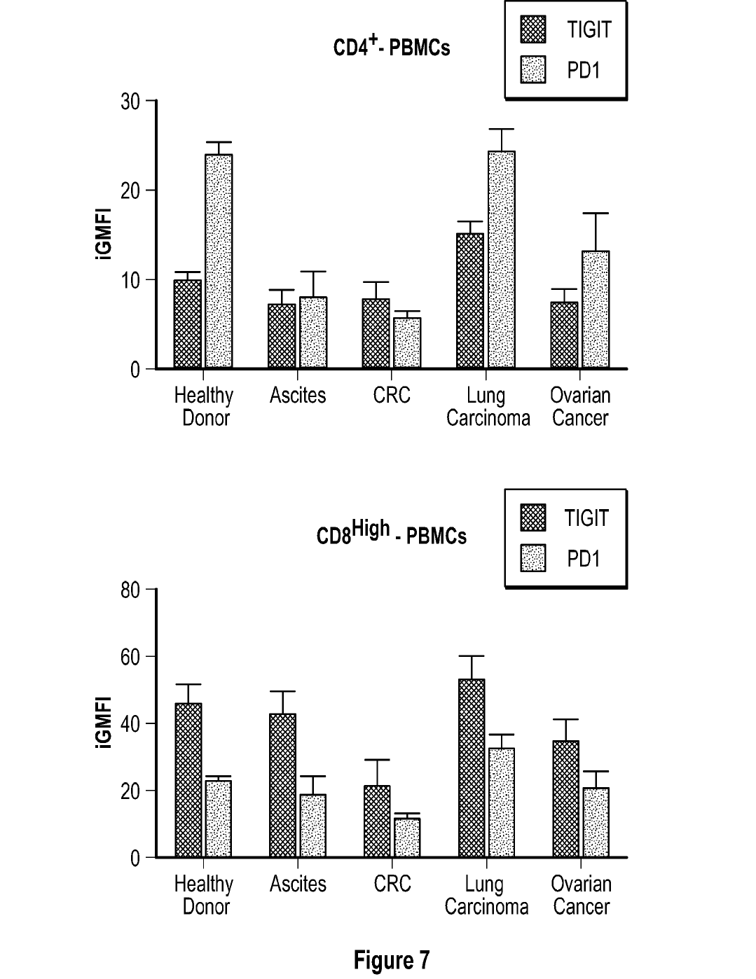

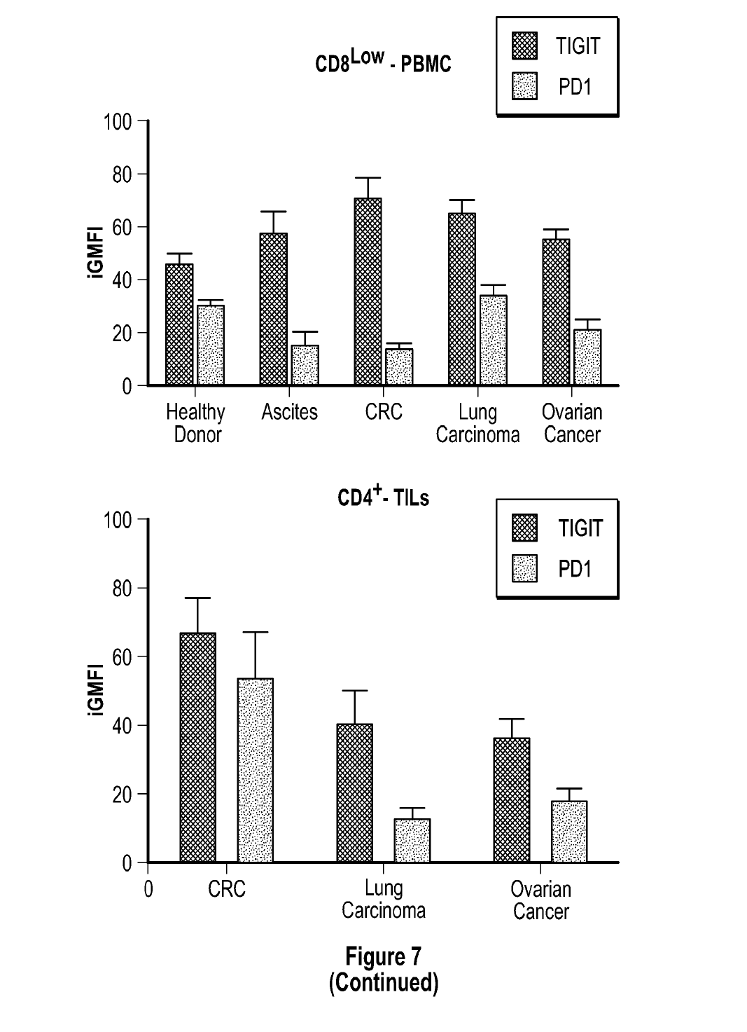

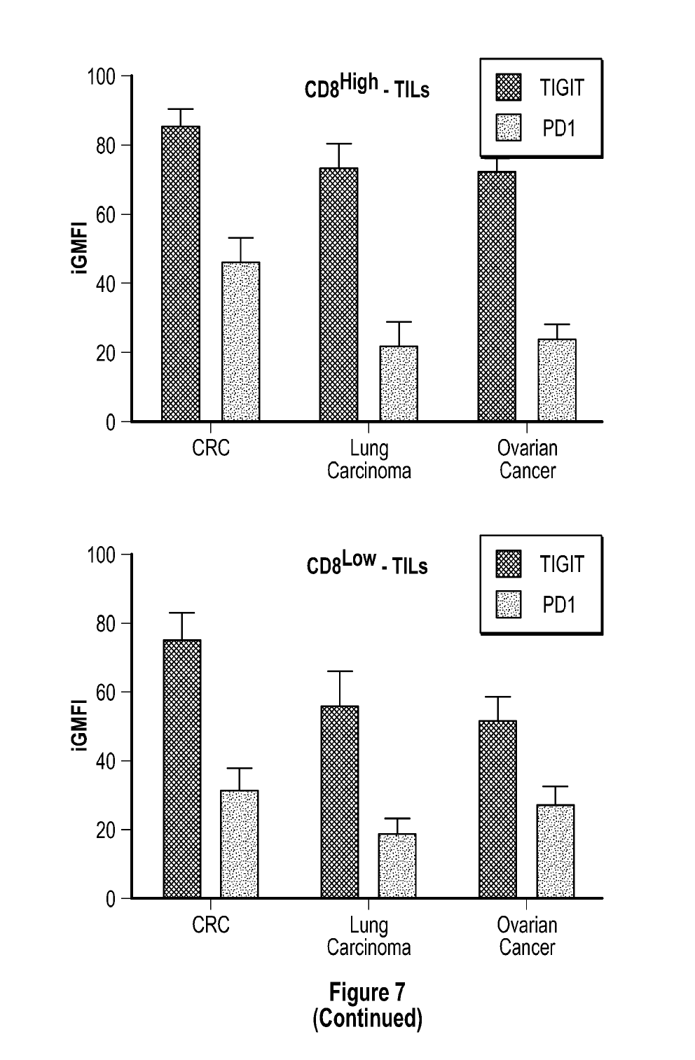

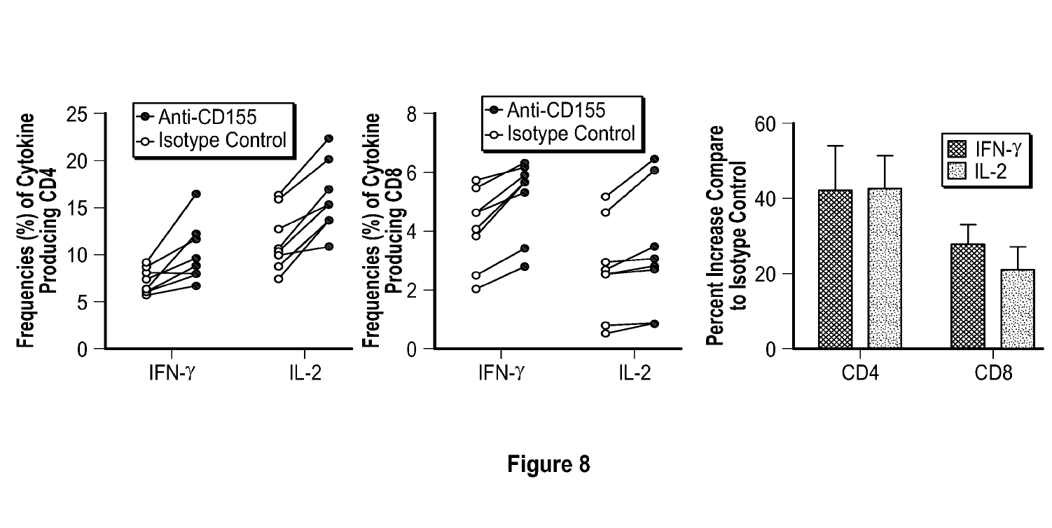

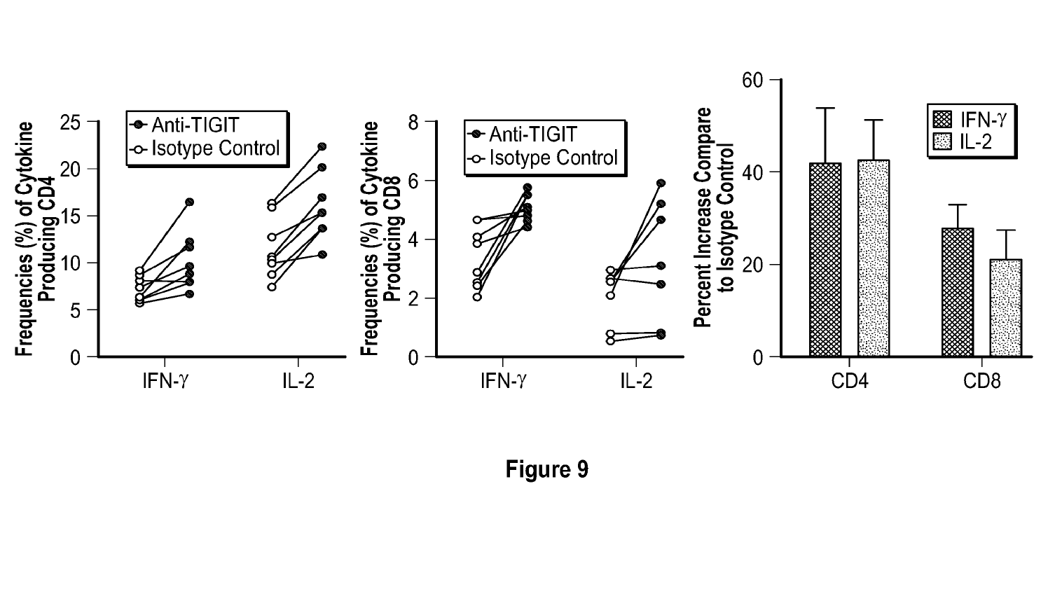

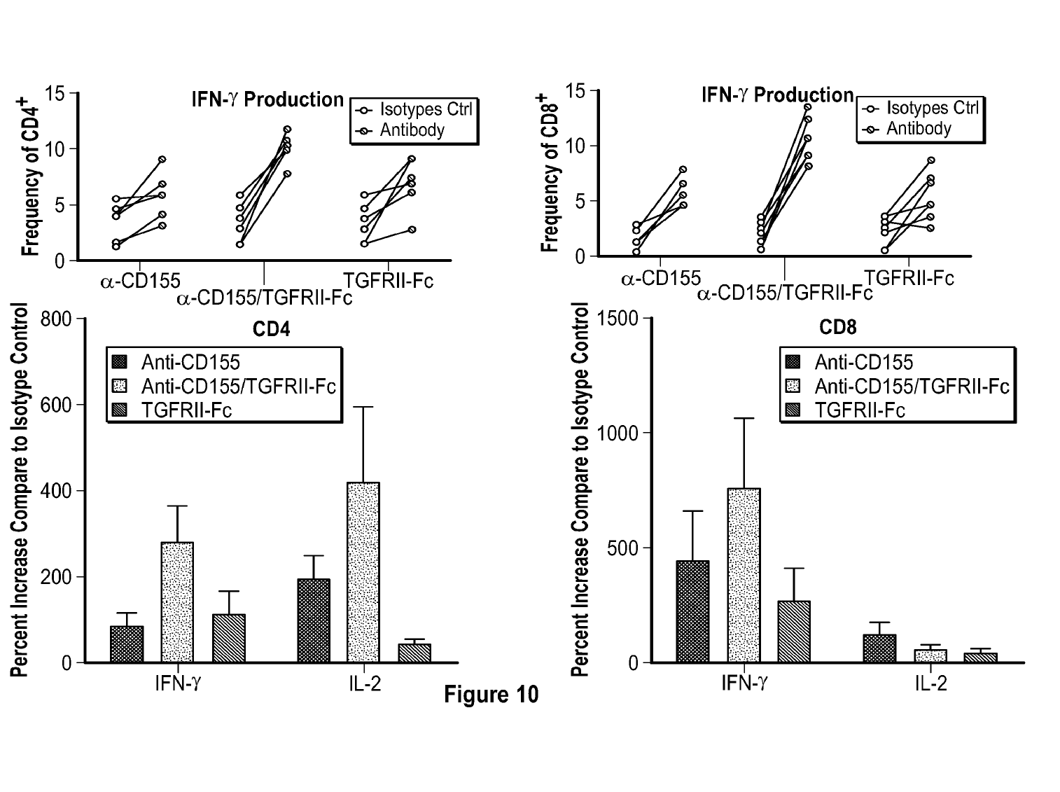

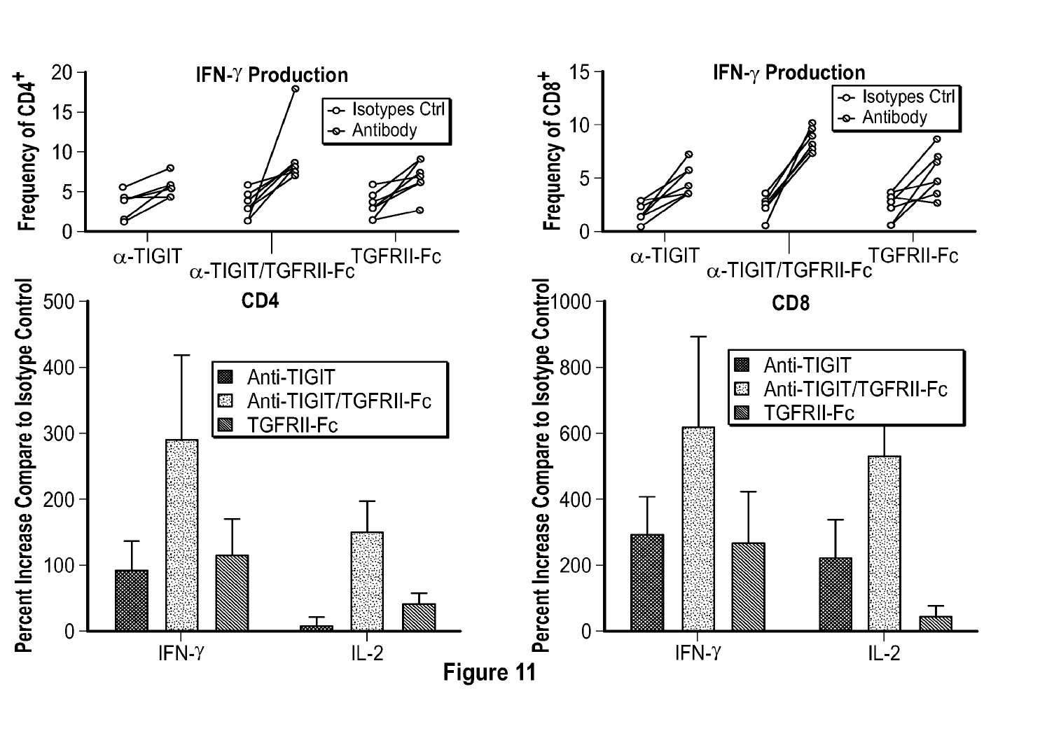

The column segments depicted in FIG. 2A, which represent gene alteration spectra for specific cancer types, are described as follows (left to right). The first column, alterations in lung squamous cell carcinoma, features a lower segment (deletions) and an upper segment (amplifications). The second column, alterations in esophageal cancer, features a lower segment (mutations), a middle segment (deletions), and an upper segment (amplifications). The third column, alterations in head and neck cancer, features a lower segment (mutations) and an upper segment (amplifications). The fourth column, alterations in ovarian cancer, is non-segmented column (amplifications). The fifth column, alterations in non-small cell lung cancer, features a lower segment (mutations), a middle segment (deletions), and an upper segment (amplifications). The sixth column, alterations in cholangiocarcinoma, is non-segmented column (mutations). The seventh column, alterations in cervical cancer, features a lower segment (mutations) and an upper segment (amplifications). The eighth column, alteration in sarcomas, features a lower segment (deletions) and an upper segment (amplifications). The ninth column, alterations in lung adenocarcinoma, features a lower segment (mutations) and an upper segment (amplifications). The tenth column, alterations in diffuse large B cell lymphoma, is a non-segmented column (amplifications). The eleventh column, alterations in uterine cancer, features a lower segment (mutations) and an upper segment (deletions). The twelfth column, alterations in colorectal cancer, is a non-segmented column (mutations). The thirteenth column, alterations in small cell lung cancer, is a non-segmented column (mutations). The fourteenth column, alterations in melanoma, is a non-segmented column (mutations). The fifteenth column, alterations in breast cancer, features a lower segment (mutations) and an upper segment (amplifications). The sixteenth column, alterations in bladder cancer, is a non-segmented column (amplifications). The seventeenth column, alterations in chromophobe renal cell carcinoma, is a non-segmented column (mutations). The eighteenth column, alterations in stomach cancer, features a lower segment (mutations), a middle segment (deletions), and an upper segment (amplifications). The nineteenth column, alterations in prostate cancer, features a lower segment (deletions) and an upper segment (amplifications). The twentieth column, alterations in lower grade glioma (glioblastoma), features a lower segment (deletions) and an upper segment (amplifications). The twenty-first and twenty-second columns, both to glioblastomas, are both non-segmented columns (amplifications). The twenty-third column, alterations in clear cell renal cell carcinoma, features a lower segment (deletions) and an upper segment (amplifications). The twenty-fourth column, alterations in papillary renal cell carcinoma, is a non-segmented column (mutations). The twenty-fifth column, alterations in liver cancer, is a non-segmented column (amplifications). And the twenty-sixth column, alterations in breast cancer (METABRIC data), is a non-segmented column (amplifications).

The column segments depicted in FIG. 2B, which represent gene alteration spectra for specific cancer types, are described as follows (left to right). The first column, alterations in diffuse large B cell lymphoma, features a lower segment (mutations) and an upper segment (deletions). The second column, alterations in cholangiocarcinoma, features a lower segment (mutations) and an upper segment (amplifications). The third column, alterations in bladder cancer, features a lower segment (mutations) and an upper segment (amplifications). The fourth column, alterations in ovarian cancer, features a lower segment (deletions) and an upper segment (amplifications). The fifth column, alterations in lung squamous cell carcinoma, features a lower segment (mutations) and an upper segment (amplifications). The sixth column, alterations in stomach cancer, features a lower segment (mutations), a middle segment (deletions), and an upper segment (amplifications). The seventh column, alterations in cervical cancer, features a lower segment (mutations) and an upper segment (amplifications). The eighth column, alterations in lung adenocarcinoma, is a non-segmented column (amplifications). The ninth column, alterations in adenoid cystic carcinoma, features a lower segment (mutations) and an upper segment (amplifications). The tenth column, alterations in esophageal cancer, features a lower segment (mutations), a middle segment (deletions), and an upper segment (amplifications). The eleventh column, alterations in sarcomas, features a lower segment (deletions) and upper segment (amplifications). The twelfth column, alterations in liver cancer, features a lower segment (mutations) and an upper segment (amplifications). The thirteenth column, alterations in non-small cell lung carcinoma, features a small lower segment (multiple alterations), a middle segment (mutations), and an upper segment (amplifications). The fourteenth column, alterations in cutaneous T cell lymphoma, is a non-segmented column (mutations). The fifteenth column, alterations in mesothelioma, is a non-segmented column (amplifications). The sixteenth column, alterations in uterine cancer, features a lower segment (mutations) and the upper segment (amplifications). The seventeenth column, alterations in breast cancer, features a lower segment (mutations), a middle segment (deletions), and an upper segment (amplifications). The eighteenth column, alterations in thymomas, is a non-segmented column (mutations). The nineteenth column, alterations in lower grade glioma (glioblastoma), features a lower segment (mutations), a middle segment (deletions), and an upper segment (amplifications). The twentieth column, alterations in melanoma, features a lower segment (mutations) and an upper segment (amplifications). The twenty-first column, alterations in head and neck cancers, also features a lower segment (mutations) and an upper segment (amplifications). The twenty-second column, alterations in uveal melanoma, is a non-segmented column (mutations). The twenty-third column, alterations in prostate cancer, features a lower segment (deletions) and an upper segment (amplifications). The twenty-fourth column, alterations in glioblastoma, features a lower segment (mutations) and an upper segment (amplifications). The twenty-fifth column, alterations in non-clear cell renal cell carcinoma, is a non-segmented column (mutations). The twenty-sixth column, alterations in glioblastoma, features a lower segment (mutations) and an upper segment (amplifications). The twenty-seventh column, alterations in colorectal cancer, is a non-segmented column (mutations). The twenty-eighth column, alterations in papillary renal cell carcinoma, is a non-segmented column (deletions). The twenty-ninth column, alterations in breast cancer (METABRIC data), is a non-segmented column (amplifications). The thirtieth column, alterations in pancreatic cancer, is a non-segmented column (mutations). The thirty-first column, alterations in clear cell renal cell carcinoma, is a non-segmented column (amplifications). And the thirty-second column, alterations in chronic lymphocytic leukemia, is a non-segmented column (mutations).

The column segments depicted in FIG. 2C, which represent gene alteration spectra for specific cancer types, are described as follows (left to right). The first column, alterations in sarcomas, features a lower segment (mutations) and an upper segment (amplifications). The second column, alterations in ovarian cancer, features a lower segment (deletions) and an upper segment (amplifications). The third column, alterations in bladder cancer, is a non-segmented column (amplifications). The fourth column, alterations in lung squamous cell carcinoma, features a lower segment (mutations) and an upper segment (amplifications). The fifth column, alterations in cholangiocarcinoma, is a non-segmented column (amplifications). The sixth column, alterations in stomach cancer, features a lower segment (mutations), a middle segment (deletions), and an upper segment (amplifications). The seventh column, alterations in mesothelioma, is a non-segmented column (amplifications). The eighth column, alterations in breast cancer, features a lower segment (mutations), a middle segment (deletions), and an upper segment (amplifications). The ninth column, alterations in non-small cell lung cancer, features a lower segment (mutations), a middle segment (deletions), and an upper segment (amplifications). The tenth column, alterations in lung adenocarcinoma, features a lower segment (mutations) and an upper segment (amplifications). The eleventh column, alterations in uterine cancer, features a lower segment (mutations) and an upper segment (amplifications). The twelfth column, alterations in cervical cancer, is a non-segmented column (amplifications). The thirteenth column, alterations in melanoma, features a lower segment (mutations) and an upper segment (amplifications). The fourteenth column, alterations in liver cancer, features a lower segment (mutations) and an upper segment (amplifications). The fifteenth column, alterations in colorectal cancer, features a lower segment (mutations) and an upper segment (amplifications). The sixteenth column, alterations in lower grade glioma (glioblastoma), features a lower segment (deletions) and an upper segment (amplifications). The seventeenth column, alterations in esophageal cancer, features a lower segment (deletions) and an upper segment (amplifications). The eighteenth column, alterations in prostate cancer, features a lower portion (mutations) and an upper segment (deletions). The nineteenth column, alterations in small cell lung cancer, is a non-segmented column (mutations). The twentieth column, alterations in head and neck cancer, features a lower segment (mutations) and an upper segment (amplifications). The twenty-first column, alterations in testicular germ cell cancer, is a non-segmented column (mutations). The twenty-second column, alterations in multiple myeloma, is a non-segmented column (mutations). The twenty-third column, alterations in papillary renal cell carcinoma, is a non-segmented column (deletions). The twenty-fourth column, alterations in glioblastoma, is a non-segmented column (deletions). The twenty-fifth column, alterations in clear cell renal carcinoma, is a non-segmented column (amplifications). The twenty-sixth column, alterations in chronic lymphocytic leukemia, is a non-segmented column (mutations). And the twenty-seventh column, alterations in breast cancer (METABRIC data), is a non-segmented column (amplifications).

FIG. 3 shows bar graphs indicating the relative expression of PDL1, CD155, TIGIT, PD1 and TGF-β mRNA measured in cancer patients compared to healthy individuals, represented as the z-score of mRNA expression. Each bar represents the mean z-score from each study, with the sample size indicated in parenthesis and the error bars representing the standard error of the mean.

FIG. 4 shows plots indicating the expression of CD155 in various cancer cell lines as measured by flow cytometry.

FIG. 5 is a bar graph comparing expression of CD155 to PDL1 on tumor cells (CD45-EpCAM+), immune cells (CD45+ EpCAM−) and stroma cells (CD45− EpCAM−) isolated from ovarian cancer patient ascites, as measured by integrated geometric mean fluorescence (iGMFI).

FIG. 6 is a bar graph comparing expression of CD155 to PDL1 on tumor cells (CD45− EpCAM+) isolated from dissociated tumor cells from patients with colon rectal cancer (CRC), lung carcinoma or ovarian cancer, as measured by integrated geometric mean fluorescence (iGMFI).

FIG. 7 shows bar graphs comparing expression of TIGIT to PD1 on various cell types isolated from dissociated tumor cells and PBMCs from healthy donors and patients with CRC, lung carcinoma or ovarian cancer, as measured by integrated geometric mean fluorescence (iGMFI).

FIG. 8 shows the frequency of cytokine (IFN-γ or IL-2) producing CD4+ and CD8+ T cells when PBMCs from healthy donors were co-cultured with MDA-MB31 cells (breast adenocarcinoma) treated with an anti-CD155 antibody or isotype control.

FIG. 9 shows the frequency of cytokine (IFN-γ or IL-2) producing CD4+ and CD8+ T cells when PBMCs from healthy donors were co-cultured with MDA-MB31 cells (breast adenocarcinoma) treated with an anti-TIGIT antibody or isotype control.

FIG. 10 shows the frequency of IFN-γ producing CD4+ and CD8+ T cells (top) and percent increase of cytokine production (IFN-γ and IL-2) from CD4+ and CD8+ T cells (bottom) when PBMCs from healthy donors were co-cultured with MDA-MB31 cells (breast adenocarcinoma) treated with an anti-CD155 antibody, or TGF-β receptor II fragment conjugated to an Fc (TGFRII-Fc), alone or in combination. Each triplet of columns in the bar graphs are, left to right, Anti-CD155, Anti-CD155/TGFRII-Fc, and TGFRII-Fc, respectively.

FIG. 11 shows the frequency of IFN-γ producing CD4+ and CD8+ T cells (top) and percent increase of cytokine production (IFN-γ and IL-2) from CD4+ and CD8+ T cells (bottom) when PBMCs from healthy donors were co-cultured with MDA-MB31 cells (breast adenocarcinoma) treated with an anti-TIGIT antibody, or TGF-β receptor II fragment conjugated to an Fc (TGFRII-Fc), alone or in combination. Each triplet of columns in the bar graphs are, left to right, Anti-TIGIT, Anti-TIGIT/TGFRII-Fc, and TGFRII-Fc, respectively.

### DETAILED DESCRIPTION

### Overview

Various diseases are characterized by the development of progressive immunosuppression in a patient. The presence of an impaired immune response in patients with malignancies has been particularly well documented. Cancer patients exhibit a variety of altered immune functions such as a decrease in delayed hypersensitivity, and decrease in lytic function and proliferation response of lymphocytes. Augmenting immune functions in cancer patients may have beneficial effects for tumor control.

In one aspect, the present disclosure relates to a method of treating cancer in a patient comprising administering an effective amount of a CD155/TIGIT antagonist and a TGF-β1 antagonist, thereby treating the patient. Each agent individually targets the immune system and combined treatment (e.g., simultaneous, sequential, concurrent, prior and subsequent administration) provides improved effects in cancer patients in need thereof. Thus, in another aspect, the present disclosure relates to a method of enhancing a cancer-specific immune response in a cancer patient, such as a T cell response (e.g., CD4+ and/or CD8+ T cell response) by administering an effective amount of a CD155/TIGIT antagonist and a TGF-β1 antagonist. Treatment herein includes administration of an effective amount of a CD155/TIGIT antagonist to a patient who has received or is receiving treatment with a TGF-β1 antagonist, or administration of an effective amount of a TGF-β1 antagonist to a patient who has received or is receiving treatment with a CD155/TIGIT antagonist.

### Definitions

Terms used in the claims and specification are defined as set forth below unless otherwise specified. In the case of direct conflict with a term used in a parent provisional patent application, the term used in the instant application shall control.

It must be noted that, as used in the specification and the appended claims, the singular forms “a,” “an” and “the” include plural referents unless the context clearly dictates otherwise.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The term “ameliorating” refers to any therapeutically beneficial result in the treatment of a disease state, e.g., cancer, including prophylaxis, lessening in the severity or progression, remission, or cure thereof.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups 1 e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

As used herein, an “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, larger “peptide insertions,” can also be made, e.g. insertion of about three to about five or even up to about ten, fifteen, or twenty amino acid residues. The inserted residue(s) may be naturally occurring or non- naturally occurring as disclosed above. An “amino acid deletion” refers to the removal of at least one amino acid residue from a predetermined amino acid sequence.

As used herein, the term “antagonist” refers to any molecule that partially or fully blocks, inhibits, or neutralizes a biological activity of a native polypeptide disclosed herein. Suitable antagonist molecules specifically include antagonist antibodies or antibody fragments, fragments or amino acid sequence variants of native polypeptides, peptides, antisense oligonucleotides, small organic molecules, etc. In some embodiments, inhibition in the presence of the antagonist is observed in a dose-dependent manner. In some embodiments, the measured signal (e.g., biological activity) is at least about 5% , at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% lower than the signal measured with a negative control under comparable conditions. Also disclosed herein, are methods of identifying antagonists suitable for use in the methods of the disclosure. For example, these methods include, but are not limited to, binding assays such as enzyme-linked immuno-absorbent assay (ELISA), Forte Bio© systems, and radioimmunoas say (RIA). These assays determine the ability of an antagonist to bind the polypeptide of interest (e.g., a receptor or ligand) and therefore indicate the ability of the antagonist to inhibit, neutralize or block the activity of the polypeptide. Efficacy of an antagonist can also be determined using functional assays, such as the ability of an antagonist to inhibit the function of the polypeptide or an agonist. For example, a functional assay may comprise contacting a polypeptide with a candidate antagonist molecule and measuring a detectable change in one or more biological activities normally associated with the polypeptide. The potency of an antagonist is usually defined by its IC₅₀ value (concentration required to inhibit 50% of the agonist response). The lower the IC₅₀ value the greater the potency of the antagonist and the lower the concentration that is required to inhibit the maximum biological response.

As used herein, the term “antibody” refers to a whole antibody comprising two light chain polypeptides and two heavy chain polypeptides. Whole antibodies include different antibody isotypes including IgM, IgG, IgA, IgD, and IgE antibodies. The term “antibody” includes a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody.

As used herein, the term “antibody fragment,” “antigen-binding fragment,” or similar terms refer to a fragment of an antibody that retains the ability to bind to a target antigen (e.g., CD155, TIGIT or TGF-β1) and inhibit the activity of the target antigen. Such fragments include, e.g., a single chain antibody, a single chain Fv fragment (scFv), an Fd fragment, an Fab fragment, an Fab′ fragment, or an F(ab′)₂ fragment. A scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al. (2001) J Immunol Methods 248(1):47-66; Hudson and Kortt (1999) J Immunol Methods 231(1):177-189; Poljak (1994) Structure 2(12):1121-1123; Rondon and Marasco (1997) Annual Review of Microbiology 51:257-283, the disclosures of each of which are incorporated herein by reference in their entirety.

In some embodiments, an antigen-binding fragment includes the variable region of a heavy chain polypeptide and the variable region of a light chain polypeptide. In some embodiments, an antigen-binding fragment described herein comprises the CDRs of the light chain and heavy chain polypeptide of an antibody.

As used herein, the term “bispecific” or “bifunctional antibody” refers to an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

Traditionally, the recombinant production of bispecific antibodies is based on the co-expression of two immunoglobulin heavy-chain/light-chain pairs, where the two heavy chain/light-chain pairs have different specificities (Milstein and Cuello (1983) Nature 305:537-539). Antibody variable domains with the desired binding specificities (antibody-antigen combining sites) can be fused to immunoglobulin constant domain sequences. The fusion of the heavy chain variable region is preferably with an immunoglobulin heavy-chain constant domain, including at least part of the hinge, CH2, and CH3 regions. For further details of illustrative currently known methods for generating bispecific antibodies see, e.g., Suresh et al. (1986) Methods in Enzymology 121:210; PCT Publication No. WO 96/27011; Brennan et al. (1985) Science 229:81; Shalaby et al., J Exp Med (1992) 175:217-225; Kostelny et al. (1992) J Immunol 148(5):1547-1553; Hollinger et al. (1993) Proc Natl Acad Sci USA 90:6444-6448; Gruber et al. (1994) J Immunol 152:5368; and Tutt et al. (1991) J Immunol 147:60. Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. See, e.g., Kostelny et al. (1992) J Immunol 148(5):1547-1553. The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. The “diabody” technology described by Hollinger et al. (1993) Proc Natl Acad Sci USA 90:6444-6448 has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (scFv) dimers has also been reported. See, e.g., Gruber et al. (1994) J Immunol 152:5368. Alternatively, the antibodies can be “linear antibodies” as described in, e.g., Zapata et al. (1995) Protein Eng. 8(10):1057-1062. Briefly, these antibodies comprise a pair of tandem Fd segments (VH-CH1-VH-CH1) which form a pair of antigen binding regions. Linear antibodies can be bispecific or monospecific.

Antibodies with more than two valencies (e.g., trispecific antibodies) are contemplated and described in, e.g., Tutt et al. (1991) J Immunol 147:60.

The disclosure also embraces variant forms of multi-specific antibodies such as the dual variable domain immunoglobulin (DVD-Ig) molecules described in Wu et al. (2007) Nat Biotechnol 25(11): 1290-1297. The DVD-Ig molecules are designed such that two different light chain variable domains (VL) from two different parent antibodies are linked in tandem directly or via a short linker by recombinant DNA techniques, followed by the light chain constant domain. Similarly, the heavy chain comprises two different heavy chain variable domains (VH) linked in tandem, followed by the constant domain CH1 and Fc region. Methods for making DVD-Ig molecules from two parent antibodies are further described in, e.g., PCT Publication Nos. WO 08/024188 and WO 07/024715. In some embodiments, the bispecific antibody is a Fabs-in-Tandem immunoglobulin, in which the light chain variable region with a second specificity is fused to the heavy chain variable region of a whole antibody. Such antibodies are described in, e.g., International Patent Application Publication No. WO 2015/103072.

In some embodiments, an antagonist described herein is a nanobody, such as a camelid or dromedary antibodies (e.g., antibodies derived from Camelus bactrianus, Calelus dromaderius, or Lama paccos). Such antibodies, unlike the typical two-chain (fragment) or four-chain (whole antibody) antibodies from most mammals, generally lack light chains. See U.S. Pat. No. 5,759,808; Stijlemans et al. (2004) J Biol Chem 279:1256-1261; Dumoulin et al. (2003) Nature 424:783-788; and Pleschberger et al. (2003) Bioconjugate Chem 14:440-448. As with other antibodies of non-human origin, an amino acid sequence of a camelid antibody can be altered recombinantly to obtain a sequence that more closely resembles a human sequence, i.e., the nanobody can be “humanized” to thereby further reduce the potential immunogenicity of the antibody.

In some embodiments, an antagonist described herein is a non-antibody, scaffold protein. These proteins are, generally, obtained through combinatorial chemistry-based adaptation of preexisting antigen-binding proteins. For example, the binding site of human transferrin for human transferrin receptor can be diversified using the system described herein to create a diverse library of transferrin variants, some of which have acquired affinity for different antigens. Ali et al. (1999) J Biol Chem 274:24066-24073. The portion of human transferrin not involved with binding the receptor remains unchanged and serves as a scaffold, like framework regions of antibodies, to present the variant binding sites. The libraries are then screened, as an antibody library is, and in accordance with the methods described herein, against a target antigen of interest to identify those variants having optimal selectivity and affinity for the target antigen. Hey et al. (2005) TRENDS Biotechnol 23(10):514-522.

One of skill in the art would appreciate that the scaffold portion of the non-antibody scaffold protein can include, e.g., all or part of: the Z domain of S. aureus protein A, human transferrin, human tenth fibronectin type III domain, kunitz domain of a human trypsin inhibitor, human CTLA-4, an ankyrin repeat protein, a human lipocalin (e.g., anticalins), human crystallin, human ubiquitin, or a trypsin inhibitor from E. elaterium.

As used herein, we may use the terms “cancer” (or “cancerous”), “hyperproliferative,” and “neoplastic” to refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

As used herein, “cancer antigen” refers to (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

As used herein, the term “cancer-specific immune response” refers to the immune response induced by the presence of tumors, cancer cells, or cancer antigens. In certain embodiments, the response includes the proliferation of cancer antigen specific lymphocytes. In certain embodiments, the response includes expression and upregulation of antibodies and T-cell receptors and the formation and release of lymphokines, chemokines, and cytokines. Both innate and acquired immune systems interact to initiate antigenic responses against the tumors, cancer cells, or cancer antigens. In certain embodiments, the cancer-specific immune response is a T cell response.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The CD155/TIGIT antagonist (e.g., anti-CD155 antibody or anti-TIGIT antibody) and/or TGF-β1 antagonist (e.g., a soluble form of a TGF-β receptor protein), can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

As used herein, the term “CD155” refers to a member of the immunoglobulin superfamily and is a Type I transmembrane glygoprotein highly expressed on dendritic cells, fibroblasts, endothelial cells and some tumor cells. In some embodiments, CD155 binds with high affinity to TIGIT.

As used herein, the term “CD155/TIGIT antagonist” refers to a molecule capable of inhibiting the signaling pathway induced by binding of CD155 to TIGIT. In certain embodiments, a CD155/TIGIT antagonist specifically antagonizes CD155. In certain embodiments, the CD155/TIGIT antagonist disrupts the interaction of CD155 with TIGIT. In certain embodiments, a CD155/TIGIT antagonist is an anti-CD155 antibody. In certain embodiments, a CD155/TIGIT antagonist specifically antagonizes TIGIT. In certain embodiments, the CD155/TIGIT antagonist disrupts the interaction of TIGIT with CD155. In certain embodiments, a CD155/TIGIT antagonist is an anti-TIGIT antibody.

As used herein, “combination therapy” refers to administration of each active agent or therapy in a regimen that will provide beneficial effects of the combination, including co-administration of active agents or therapies in a substantially simultaneous manner, such as in a single formulation having a fixed ratio of active agents or in co-administration in a sequential manner, such as in multiple, separate formulations of each active agent or therapy. Combination therapy also includes combinations where individual elements may be administered at different times and/or by different routes but which act in combination to provide a beneficial effect by co-action or pharmacokinetic and pharmacodynamics effect of each agent or treatment approaches of the combination therapy.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from another peptide may have one or more mutations relative to the starting polypeptide, e.g., one or more amino acid residues which have been substituted with another amino acid residue or which has one or more amino acid residue insertions or deletions. A polypeptide can comprise an amino acid sequence which is not naturally occurring. Such variants necessarily have less than 100% sequence identity or similarity with the starting molecule. In certain embodiments, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In certain embodiments, there is one amino acid difference between a starting polypeptide sequence and the sequence derived there from. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity. In certain embodiments, a polypeptide consists of, consists essentially of, or comprises an amino acid sequence selected from SEQ ID NOs: 1-5. In certain embodiments, a polypeptide includes an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence selected from SEQ ID NOs: 1-5. In certain embodiments, a polypeptide includes a contiguous amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous amino acid sequence selected from SEQ ID NOs: 1-5. In certain embodiments, a polypeptide includes an amino acid sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous amino acids of an amino acid sequence selected from SEQ ID NOs: 1-5.

In certain embodiments, the peptides of the invention are encoded by a nucleotide sequence. Nucleotide sequences of the invention can be useful for a number of applications, including: cloning, gene therapy, protein expression and purification, mutation introduction, DNA vaccination of a host in need thereof, antibody generation for, e.g., passive immunization, PCR, primer and probe generation, and the like. In certain embodiments, the nucleotide sequence of the invention comprises, consists of, or consists essentially of, a nucleotide sequence selected from SEQ ID NO: 6. In certain embodiments, a nucleotide sequence includes a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence set forth in SEQ ID NO: 6. In certain embodiments, a nucleotide sequence includes a contiguous nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous nucleotide sequence set forth in SEQ ID NO: 6. In certain embodiments, a nucleotide sequence includes a nucleotide sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous nucleotides of a nucleotide sequence set forth in SEQ ID NO: 6.

It will also be understood by one of ordinary skill in the art that the polypeptides (e.g., CD155/TIGIT antagonist, TGF-β receptor protein) suitable for use in the methods disclosed herein may be altered such that they vary in sequence from the naturally occurring or native sequences from which they were derived, while retaining the desirable activity of the native sequences. For example, nucleotide or amino acid substitutions leading to conservative substitutions or changes at “non-essential” amino acid residues may be made. Mutations may be introduced by standard techniques, such as site-directed mutagenesis and PCR-mediated mutagenesis.

The polypeptides suitable for use in the methods disclosed herein may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In certain embodiments, a string of amino acids can be replaced with a structurally similar string that differs in order and/or composition of side chain family members. Alternatively, in certain embodiments, mutations may be introduced randomly along all or part of a coding sequence, such as by saturation mutagenesis, and the resultant mutants can be incorporated into binding polypeptides of the invention and screened for their ability to bind to the desired target.

As used herein, the term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from, for example, CD155 or TIGIT, are tested for reactivity with a given anti-CD155 antibody or anti-TIGIT antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

As used herein, the term “Fc region” refers to the portion of a native immunoglobulin formed by the respective Fc domains (or Fc moieties) of its two heavy chains. As used herein, the term “Fc domain” refers to a portion of a single immunoglobulin (Ig) heavy chain wherein the Fc domain does not comprise an Fv domain. As such, an Fc domain can also be referred to as “Ig” or “IgG.” In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. A human IgG1 constant region can be found at Uniprot P01857 and in Table 1 (i.e., SEQ ID NO: 1). The Fc domain of human IgG1 can be found in Table 1 (i.e., SEQ ID NO: 2). The Fc domain encompasses native Fc and Fc variant molecules. As with Fc variants and native Fc's, the term Fc domain includes molecules in monomeric or multimeric form, whether digested from whole antibody or produced by other means. The assignment of amino acid residue numbers to an Fc domain is in accordance with the definitions of Kabat. See, e.g., Sequences of Proteins of Immunological Interest (Table of Contents, Introduction and Constant Region Sequences sections), 5th edition, Bethesda, Md.: NIH vol. 1:647-723 (1991); Kabat et al., “Introduction” Sequences of Proteins of Immunological Interest, US Dept of Health and Human Services, NIH, 5th edition, Bethesda, Md. vol. 1:xiii-xcvi (1991); Chothia & Lesk, J. Mol. Biol. 196:901-917 (1987); Chothia et al., Nature 342:878-883 (1989), each of which is herein incorporated by reference for all purposes.

As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding).

The Fc domains of a polypeptide of the disclosure may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

As used herein, the term “gly-ser polypeptide linker” refers to a peptide that consists of glycine and serine residues. An exemplary gly-ser polypeptide linker comprises the amino acid sequence Ser(Gly₄Ser)n. In certain embodiments, n=1. In certain embodiments, n=2. In certain embodiments, n=3, i.e., Ser(Gly₄Ser)3. In certain embodiments, n=4, i.e., Ser(Gly₄Ser)4. In certain embodiments, n=5. In certain embodiments, n=6. In certain embodiments, n=7. In certain embodiments, n=8. In certain embodiments, n=9. In certain embodiments, n=10. Another exemplary gly-ser polypeptide linker comprises the amino acid sequence (Gly₄Ser)n. In certain embodiments, n=1. In certain embodiments, n=2. In certain embodiments, n=3. In certain embodiments, n=4. In certain embodiments, n=5. In certain embodiments, n=6. Another exemplary gly-ser polypeptide linker comprises the amino acid sequence (Gly₃Ser)n. certain embodiments, n=1. In certain embodiments, n=2. In certain embodiments, n=3. In certain embodiments, n=4. In certain embodiments, n=5. In certain embodiments, n=6.

As used herein, “half-life” refers to the time taken for the serum or plasma concentration of a polypeptide to reduce by 50%, in vivo, for example due to degradation and/or clearance or sequestration by natural mechanisms. The polypeptides (e.g., CD155/TIGIT antagonist and/or TGF-β1 antagonist) suitable for use in the methods disclosed herein is stabilized in vivo and its half-life increased by, e.g., fusion to an Fc region, fusion to serum albumin (e.g., HSA or MSA), through PEGylation, or by binding to serum albumin molecules (e.g., human serum albumin) which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

In certain aspects, the TGF-β1 antagonist comprising a moiety that enhances the serum half-life of the TGF-β1 antagonist, suitable for use in the methods disclosed herein, can employ one or more “linker domains,” such as polypeptide linkers. As used herein, the term “linker” or “linker domain” refers to a sequence which connects two or more domains (e.g., the serum half-life enhancing moiety and TGF-β receptor protein) in a linear sequence. As used herein, the term “polypeptide linker” refers to a peptide or polypeptide sequence (e.g., a synthetic peptide or polypeptide sequence) which connects two or more domains in a linear amino acid sequence of a polypeptide chain. For example, polypeptide linkers may be used to couple a TGF-β receptor protein (e.g., extracellular domain of TGFβRII) to an Fc domain. Preferably, such polypeptide linkers can provide flexibility to the polypeptide molecule. In certain embodiments the polypeptide linker is used to couple (e.g., genetically fuse) one or more Fc domains and/ TGF-β receptor protein (e.g., extracellular domain of TGFβRII).

As used herein, the term a “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

As used herein, the term “human antibody” includes antibodies having variable and constant regions (if present) of human germline immunoglobulin sequences. Human antibodies of the disclosure can include amino acid residues not encoded by human germline immunoglobulin sequences (e.g., mutations introduced by random or site-specific mutagenesis in vitro or by somatic mutation in vivo) (see, Lonberg, N. et al. (1994) Nature 368(6474): 856-859); Lonberg, N. (1994) Handbook of Experimental Pharmacology 113:49-101; Lonberg, N. and Huszar, D. (1995) Intern. Rev. Immunol. Vol. 13: 65-93, and Harding, F. and Lonberg, N. (1995) Ann. N.Y. Acad. Sci 764:536-546). However, the term “human antibody” does not include antibodies in which CDR sequences derived from the germline of another mammalian species, such as a mouse, have been grafted onto human framework sequences (i.e., humanized antibodies).

As used herein, “immune cell” is a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).

As used herein, the terms “inhibits” or “blocks” (e.g., referring to inhibition/blocking of binding of TIGIT and CD155 or TGF-β1 to TGFβRII on cells) are used interchangeably and encompass both partial and complete inhibition/blocking. The inhibition/blocking of TIGIT, CD155 or TGF-β1 preferably reduces or alters the normal level or type of activity that occurs when TIGIT, CD155 or TGF-β1 binding occurs without inhibition or blocking. In some embodiments, the inhibition/blocking of TGF-β1 is accomplished by sequestering TGF-β1 (e.g., through the use of an extracellular domain of a TGF-β receptor (e.g., TGFβRII)), thereby preventing its availability, and thus ability, to bind a TGF-β receptor (e.g., TGFβRII) present on a cell.

As used herein, the term “inhibits growth” (e.g., referring to cells) is intended to include any measurable decrease in the growth of a cell, e.g., the inhibition of growth of a cell by at least about 10%, 20%, 30%, 40%, 50%, 60%, 70%, 80%, 90%, 99%, or 100%.

The term “in vivo” refers to processes that occur in a living organism.

As used herein, the term “isolated antibody” is intended to refer to an antibody which is substantially free of other antibodies having different antigenic specificities (e.g., an isolated antibody that specifically binds to human CD155 is substantially free of antibodies that specifically bind antigens other than CD155; an isolated antibody that specifically binds to human TIGIT is substantially free of antibodies that specifically bind antigens other than TIGIT).

As used herein, “isotype” refers to the antibody class (e.g., IgM or IgG1) that is encoded by heavy chain constant region genes. In some embodiments, a human monoclonal antibody of the disclosure is of the IgG1 isotype. In some embodiments, a human monoclonal antibody of the disclosure is of the IgG2 isotype.

As used herein, the terms “linked,” “fused”, or “fusion”, are used interchangeably. These terms refer to the joining together of two more elements or components or domains, by whatever means including chemical conjugation or recombinant means. Methods of chemical conjugation (e.g., using heterobifunctional crosslinking agents) are known in the art.

The term “mammal” or “subject” or “patient” as used herein includes both humans and non-humans and includes, but is not limited to, humans, non-human primates, canines, felines, murines, bovines, equines, and porcines.

As used herein, the term “monoclonal antibody” refers to an antibody which displays a single binding specificity and affinity for a particular epitope. Accordingly, the term “human monoclonal antibody” refers to an antibody which displays a single binding specificity and which has variable and optional constant regions derived from human germline immunoglobulin sequences. In some embodiments, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

As used herein, the term “nucleic acid” refers to deoxyribonucleotides or ribonucleotides and polymers thereof in either single- or double- stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., Biol. Chem. 260:2605-2608, 1985; and Cassol et al, 1992; Rossolini et al, Mol. Cell. Probes 8:91-98, 1994). For arginine and leucine, modifications at the second base can also be conservative. The term nucleic acid is used interchangeably with gene, cDNA, and mRNA encoded by a gene.

Polynucleotides used herein can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double- stranded regions, single- and double-stranded RNA, and RNA that is mixture of single- and double-stranded regions, hybrid molecules comprising DNA and RNA that can be single- stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, the terms “overexpress,” “elevate,” and “increase” refer to the relative level of protein or gene expression of a specified target (e.g., CD155 or TIGIT). In some embodiments, the term “elevated” is equivalent to the terms “overexpress,” “increased” and similar terms. In certain embodiments, the level of protein or gene expression of a specified target in a cancer cell, tumor, or other sample from a cancer patient is considered to be overexpressed, elevated or increased if it is higher in comparison to the level of protein or gene expression in a healthy (i.e., no cancer) sample.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the terms “polypeptide,” “peptide”, and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “recombinant human antibody” includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared there from, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23(9):1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen may not have sequence identity with the original nucleic acid molecules, but instead will be substantially identical or similar (i.e., have at least 80% identity).

As used herein, the term “serum half-life enhancing moiety” or “moiety that enhances serum-half life” refers to a protein, peptide, or moiety that increases the circulation half-life of a biologically active molecule when fused to or administered together with the biologically active molecule. Examples of a serum half-life enhancing moiety include PEG, human serum albumin (HSA) binders (as disclosed in U.S. Publication Nos. 2005/0287153 and 2007/0003549, PCT Publication Nos. WO 2009/083804 and WO 2009/133208, and SABA molecules as described in US2012/094909), serum albumin (e.g., HSA), Fc or Fc fragments and variants thereof, transferrin and variants thereof, and sugars (e.g., sialic acid). Other exemplary serum half-life enhancing moieties are disclosed in Kontermann et al., Current Opinion in Biotechnology 2011; 22:868-876, which is herein incorporated by reference in its entirety. In certain embodiments, the TGF-β1 antagonist is a fusion protein in which a soluble form of TGF-β receptor protein is linked or fused to a moiety that enhances the serum half-life of the TGF-β1 antagonist. An exemplary fusion protein is a TGFβRII-Fc fusion in which the extracellular domain of TGFβRII is linked to an Fc domain.

The term “sufficient amount” or “amount sufficient to” means an amount sufficient to produce a desired effect, e.g., an amount sufficient to reduce the size of a tumor.

As used herein, “synergy” or “synergistic effect” with regard to an effect produced by two or more individual components refers to a phenomenon in which the total effect produced by these components, when utilized in combination, is greater than the sum of the individual effects of each component acting alone.

The term “T cell” refers to a CD4+ T cell or a CD8+ T cell. The term T cell encompasses TH1 cells, TH2 cells and TH17 cells.

The term “T cell cytotoxicity” includes any immune response that is mediated by CD8+ T cell activation. Exemplary immune responses include cytokine production, CD8+ T cell proliferation, granzyme or perform production, and clearance of an infectious agent.

As used herein, the term “T cell response” refers to any response of T cells, including effector T cells (e.g., CD8⁺ cells) and helper T cells (e.g., CD4⁺ cells). T cell responses include, for example, T cell proliferation and cytokine production (e.g., IL-2 and IFNγ).

As used herein, the term “TGF-β1 antagonist” refers to a molecule capable of inhibiting the binding of TGF-β1 to a TGF-β receptor, such as TGFβRII, thereby preventing signal transduction. In certain embodiments, a TGF-β1 antagonist directly binding to TGF-β1, thereby preventing binding to a TGF-β receptor. In certain embodiments, a TGF-β1 antagonist is a small molecule, nucleic acid or polypeptide. In certain embodiments, a TGF-β1 antagonist is a soluble form of a TGF-β receptor protein. In certain embodiments, a TGF-β1 antagonist is a TGF-β receptor “trap”, which refers to the use of a soluble TGF-β receptor to neutralize TGF-β1 binding. In certain embodiments, a TGF-β1 antagonist is an extracellular domain of TGFβRII or a TGF-β1 binding fragment thereof. In certain embodiments, the extracellular domain of TGFβRII comprises the amino acid sequence set forth in SEQ ID NO: 6.

In certain embodiments, a TGF-β1 antagonist is a fusion protein comprising an extracellular domain of the TGFβRII or a TGF-β1-binding fragment thereof, and a moiety that enhances the serum half-life of the TGF-β1 antagonist. In certain embodiments, a moiety that enhances the serum half life of the TGF-β1 antagonist is an Fc domain of an antibody, polyethylene glycol, or serum albumin. In certain embodiments, a TGF-β1 antagonist is a fusion protein comprising an extracellular domain of the TGFβRII or a TGF-β1-binding fragment thereof, and an IgG Fc domain. In certain embodiments, the IgG Fc domain fused to the TGF-β1 antagonist is a human IgG Fc domain, including but not limited to a human IgG1 Fc domain, a human IgG2 Fc domain, a human IgG3 Fc domain, or a human IgG4 Fc domain. In certain embodiments, a TGF-β1 antagonist is a fusion protein comprising an extracellular domain of the TGFβRII or a TGF-β1-binding fragment thereof, and a human IgG1 Fc domain. In certain embodiments, the TGF-β1 antagonist comprises (a) an extracellular domain of the type 2 TGF-β receptor comprising the amino acid sequence depicted in SEQ ID NO: 5, and (b) a human IgG1 Fc domain comprising the amino acid sequence depicted in SEQ ID NO: 2.

As used herein, the term “TGF-β pathway” refers to the signaling cascade induced by binding of one of three ligands (i.e., TGF-β1, TGF-β2 or TGF-β3) to the type II TGF-β receptor (TGFβRII). Upon ligand binding, TGFβRII recruits the type I TGF-β receptor (TGFβRI), which is then phosphorylated. Phosphorylation of TGFβRI results in the recruitment of receptors Smads (R-Smads), such as Smad-2 and Smad-3, which then bind to Smad-4. This Smad complex translocates into the nucleus and acts as a transcription factor, thereby regulating gene expression. As used herein, the term “TGF-β antagonist” refers to a molecule capable of binding to any component of the TGF-β pathway, thereby inhibiting the TGF-β signaling cascade.

As used herein, the term “therapeutic antibody” refers to an antibody, fragment of an antibody, or construct that is derived from an antibody, and can bind to a cell-surface antigen on a target cell to cause a therapeutic effect. Such antibodies can be chimeric, humanized or fully human antibodies. Methods are known in the art for producing such antibodies. Such antibodies include single chain Fc fragments of antibodies, minibodies and diabodies. Any of the therapeutic antibodies known in the art to be useful for cancer therapy can be used in the combination therapy suitable for use in the methods disclosed herein. Therapeutic antibodies may be monoclonal antibodies or polyclonal antibodies. In preferred embodiments, the therapeutic antibodies target cancer antigens.

The term “therapeutically effective amount” is an amount that is effective to ameliorate a symptom of a disease. A therapeutically effective amount can be a “prophylactically effective amount” as prophylaxis can be considered therapy.

As used herein, the term “TIGIT” or “T cell immunoreceptor with Ig and ITIM domains” refers to a 244 amino acid transmembrane protein that binds to CD155, Nectin-2 (CD112) and Nectin-3 (CD113). In some embodiments, the TIGIT-CD155 interaction negatively regulates T cell activation and proliferation.

### CD155/TIGIT Antagonists

In some aspects, the present disclosure provides methods for treating cancer by administering an antagonist of the CD155/TIGIT pathway in combination with an antagonist of the TGF-β pathway. In some embodiments, the CD155/TIGIT pathway is antagonized by targeting the molecules in the pathway individually (i.e., CD155 or TIGIT), as described infra.

### CD155

CD155, also known as Nectin-like protein 5 (Nec1-5), Tage4, HVED, and PVS was first identified as the poliovirus receptor (PVR). CD155 belongs to the immunoglobulin superfamily and is a transmembrane glycoprotein with three extracellular immunoglobulin-like domains. CD155 is highly expressed on dendritic cells (DC), FDC, fibroblasts, endothelial cells and some tumor cells. In addition, CD155 plays a role in the formation of intercellular adherens junctions between epithelial cells. (See, for example, UniProt P15151; NCBI Gene ID: 5817; US2013/0251720; Zhang et al., Proc. Natl. Acad. Sci., Vol: 105(47): 18284-18289, 2008; and Maier et al., European Journal of Immunology, Vol: 37: 2214-2225, 2007).

CD155 is capable of forming trans-heterodimers with nectin-3. The extracellular domain of CD155 interacts with CD226, CD96, and binds with high affinity to TIGIT. The extracellular domain of CD155 also mediates cell attachment to vitronectin, an extracellular matrix molecule, and the intracellular domain interacts with the dynein light chain Tctex-1/DYNLT1. (See, for example, UniProt P15151 and NCBI Gene ID: 5817).

CD155 colocalizes with integrin αᵥβ₃ at the leading edges of moving cells and enhances growth factor-induced cell movement and proliferation. However, upon cell-cell contact, CD155 is removed from the cell surface by its trans-interaction with the cell adhesion molecule nectin-3, which results in reduced movement and proliferation. (See, Morimoto et al., Oncogene, Vol. 27: 264-273, 2008).

A CD155 polypeptide may be, for example, any peptide encoded by the human gene represented by NCBI Gene ID No. 5817, such as a polypeptide having the sequences described by NCBI RefSeq No. NP_006496.

CD155 is expressed during embryonic development and is associated with cancer. Various types of tumor cells and primary tumors showing tumor cell invasion and migration express or overexpress CD155; for example, breast, colorectal carcinoma, lung adenocarcinoma, bronchial passage, epithelial lining of the gastrointestinal, upper respiratory and genitor-urinary tracts, liver, prostate, melanoma, and brain. As described in Example 1 and FIG. 3, infra, CD155 mRNA was expressed in colorectal cancer (CRC), breast cancer, cervical cancer, stomach cancer, non-small cell lung carcinoma (NSCLC), and pancreatic cancer. Interestingly, CD155 was more highly expressed that PD-L1 in all cancers analyzed. CD155 was also expressed by several cancer cell lines (See, Example 1 and FIG. 4, infra).

CD155 is also believed to function in the immune response. For example, CD155 binds two NK cell receptors, CD96 and CD226 and is believed to play a role in NK cell adhesion and trigger NK cell effector functions. (See, for example, UniProt P15151).

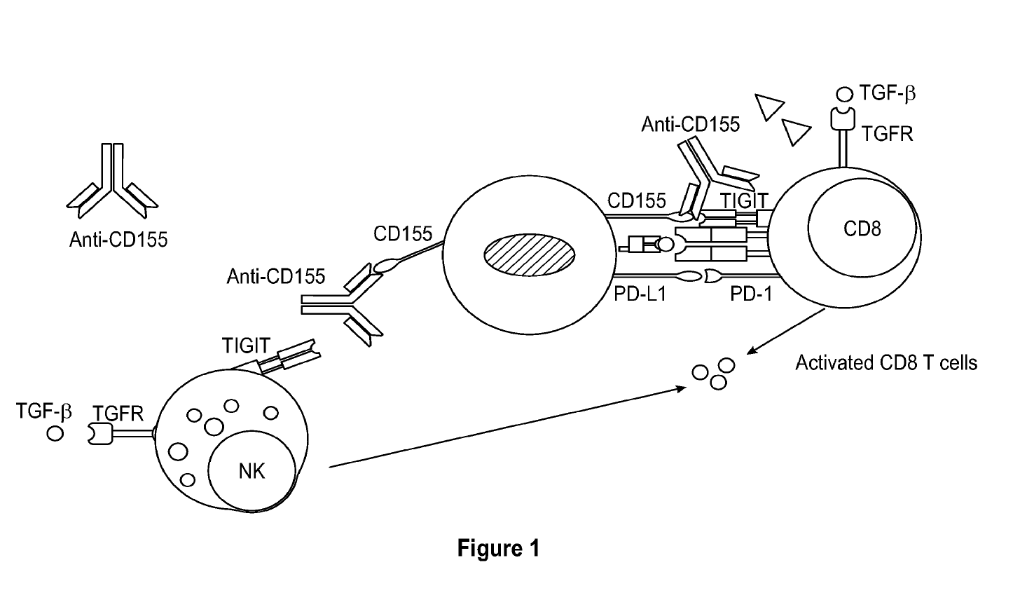

In addition, the interaction between CD155 and TIGIT (T-cell Ig and ITIM domain) triggers an immunosuppressive mechanism in melanoma. TIGIT is expressed by activated T cells, regulatory T cells (Treg), and natural killer (NK) cells, and competes with CD226 for binding to CD155. Notably, CD226 delivers activating signals into T cells; in contrast, TIGIT has immunosuppressive effects. Blockade of CD155 may, at least in part, reverse the immunosuppressive effect of this interaction. For example, blocking CD155-TIGIT signaling may enhance the effector function of cytotoxic T cells. Thus, the TIGIT-CD155 interaction, at least in part, may negatively regulate T cell activation and proliferation. (See, for example, Mahnke, et al., Journal of Investigative Dermatology, Vol. 136: 9-11, 2016; and US2013/0251720). FIG. 1 shows a schematic of blocking CD155 with an anti-CD155 antibody, thereby inducing an immune response.

The present disclosure provides the use of a CD155 antagonist (e.g., anti-CD155 antibody or antigen binding fragment thereof) to inhibit or block CD155 signaling through TIGIT in combination with an antagonist of the TGF-β pathway to induce or enhance an immune response to cancer cells in a subject. In some aspects of the disclosure, a CD155 antagonist is administered with an antagonist of the TGF-β pathway to delay disease progression, reduce or inhibit relapse or cancer progression, treat or delay progression of tumor immunity; and/or increase, enhance or stimulate an immune response or function in a cancer patient. In some embodiments, a CD155 antagonist is administered in combination with an antagonist of the TGF-β pathway to induce or enhance a cancer-specific T cell response in a patient. In some embodiments, a CD155 antagonist in combination with an antagonist of the TGF-β pathway may enhance the effect of an anti-cancer agent and/or anti-cancer therapy. In some embodiments, a CD155 antagonist in combination with an antagonist of the TGF-β pathway may promote the anti-cancer function of NK cells. In some embodiments, a CD155 antagonist in combination with an antagonist of a the TGF-β pathway may increase, enhance or stimulate an immune response or function in a cancer patient.

In some embodiments of the disclosure, anti-CD155 antibodies may be polyclonal antibodies, monoclonal antibodies, human or humanized antibodies, bispecific antibodies, or heteroconjugate antibodies. In some embodiments, the CD155 antagonist may be used in conjunction with another agent.

In some embodiments, the anti-CD155 antibody is SKII.4 or D171. (See, for example, U.S. Pat. No. 6,518,033).

In some embodiments, the anti-CD155 antibodies inhibit the binding of CD155 to TIGIT. Antibodies that bind CD155, and methods of making and using the same, are described in US2014/056890 and U.S. Pat. No. 6,518,033, the entire contents of which are incorporated herein by reference.

### TIGIT

TIGIT (T cell immunoreceptor with Ig and ITIM domains), also known as WUCAM (Washington University cell adhesion molecule), Vstm3 (V-set and transmembrane domain-containing protein 3), Vsig9 (V-set and immunoglobulin domain-containing protein 9), and PRO52254, is a 244 amino acid transmembrane protein. TIGIT was identified as a cell surface bound protein with an IgV domain, a transmembrane domain, and two putative immunoreceptor tyrosine inhibitory motifs. TIGIT functions as an immune receptor expressed by a variety of activated T cells (regulatory T cells (T_{reg}), memory T cells, and follicular B cell helper T cells (T_{fh})) and NK cells. In particular, TIGIT is highly expressed by activated T_{regs}. In addition, expression of TIGIT has been shown to be increased in arthritis, psoriasis, inflammatory bowel disorder, and breast cancer tissue relative to normal control tissue. (See for example, NCBI Gene ID: 201633, UniProt Q495A1, US2004/0121370, US2013/0251720).

A TIGIT polypeptide may be, for example, any peptide encoded by the human gene represented by NCBI Gene ID No. 201633, such as a polypeptide having the sequences described by NCBI RefSeq No. NP_776160.

TIGIT binds CD155, Nectin-2 (CD112) and Nectin-3 (CD113), and competes with CD226 (DNAM-1) and CD96 for binding to CD155 and CD112, respectively. TIGIT binds CD155 with high affinity, and is more effective at blocking the CD155-CD226 interaction than CD226 is as blocking the TIGIT-CD155 interaction. Notably, TIGIT and CD226 have opposite effects on T cells. In particular, TIGIT has been shown to have suppressive activity, whereas CD226 communicates activating signals into T cells. Furthermore, the TIGIT-CD155 interaction has been shown to negatively regulate T cell activation and proliferation. (See, for example, Mahnke, et al., Journal of Investigative Dermatology, Vol. 136: 9-11, 2016; US2013/0251720).

It has been suggested that the interaction of CD155 with CD226 may enhance the destruction of tumor cells. However, the high affinity of TIGIT for CD155 may prevent this response. Thus, the TIGIT/CD155 inhibitory interaction may function in the suppression of anti-self immune responses, but also suppress tumor destruction. (See, WO2016/106302)

Evidence also suggests that TIGIT suppresses T cell activation by promoting the generation of mature immunoregulatory dendritic cells. In addition, TIGIT, as well as other co-inhibitory molecules such as CTLA-4, PD-1, Lag3, and BTLA, may function in the evasion of immunosurveillance by tumor cells. In this regard, the TIGIT/CD155 interaction may shield tumor cells from immune-mediated destruction by inhibiting anti-tumor responses of T and NK cells. (See, WO2016/106302.)

The interaction of TIGIT with CD155 on dendritic cells (DC) is thought to modulate DC function, in particular, cytokine release. For example, TIGIT bound human DC secrete high levels of IL-10, and lower levels of pro-inflammatory and other cytokines (e.g., IL-12p40, IL-12p′70, IL-6, IL-18, and IFNγ). Furthermore, TIGIT is believed to inhibit T cell activation through an inhibitory feedback loop via the induction of IL-10 in DC. (See, US2013/0251720).

The present disclosure provides use of a TIGIT antagonist (e.g., anti-TIGIT antibody or antigen binding fragment thereof) to inhibit or block TIGIT signaling in combination with an antagonist of the TGF-β pathway to induce or enhance an immune response to cancer cells in a subject. In some aspects of the disclosure, a TIGIT antagonist is administered with an antagonist of the TGF-β pathway to delay disease progression, reduce or inhibit relapse or cancer progression, treat or delay progression of tumor immunity; and/or increase, enhance or stimulate an immune response or function in a cancer patient. In some embodiments, a TIGIT antagonist is administered in combination with an antagonist of the TGF-β pathway to induce or enhance a cancer-specific T cell response in a patient. In some embodiments, a TIGIT antagonist in combination with an antagonist of the TGF-β pathway may enhance the effect of an anti-cancer agent and/or anti-cancer therapy. In some embodiments, a TIGIT antagonist in combination with an antagonist of the TGF-β pathway may promote the anti-cancer function of NK cells. In some embodiments, a TIGIT antagonist in combination with an antagonist of the TGF-β pathway may increase, enhance or stimulate an immune response or function in a cancer patient.

In some embodiments of the disclosure, anti-TIGIT antibodies may be polyclonal antibodies, monoclonal antibodies, human or humanized antibodies, bispecific antibodies, and heteroconjugate antibodies. In some embodiments, the TIGIT antagonist may be used in conjunction with another agent. The generation of antibodies and antibody fragments that bind human TIGIT is described in US2004/0121370, WO2015/009856, WO2016/011264, WO2016/106302, and WO2016/028656 as well as methods of using these antibodies e.g., in the treatment of cancer or viral infection. The entire contents of US2004/0121370, WO2015/009856, WO2016/011264, WO2016/106302, and WO2016/028656 are incorporated herein by reference in their entirety.

In some embodiments, the anti-TIGIT antibody is 10A7, 1F4, 14A6, 28H5, 3106, 15A6, 22G2, 11G11, 10D7, MBSA43, or humanized versions thereof, as described in, e.g., WO2015/009856, WO2016/011264, WO2016/028656, and WO2016/106302, the contents of each of which are incorporated herein by reference in their entirety.

In some embodiments, anti-TIGIT antibodies inhibit the binding of TIGIT to cell surface expressed CD155. For example, the anti-TIGIT antibody may be 10A7 or 1FA, which have been observed to bind different TIGIT epitopes. Antibody 10A7 and 1FA are described in WO2015/009856, WO2016/011264, US2013/0251720, the entire contents of which are incorporated herein by reference.

TIGIT binding partners as well as compositions, detection methods, and methods of treatment of immune disorders modulated by TIGIT interaction with those binding partners, and the effects of TIGIT on T cell maturation and activity is discussed in US2013/0251720, the entire content of which is hereby incorporated by reference.

Methods for expressing TIGIT in E. coli, mammalian cells, yeast, and Baculovirus-infected insect cells, as well as methods for preparing antibodies (e.g., monoclonal, polyclonal) that bind TIGIT and methods for purifying TIGIT using anti-TIGIT antibodies are described in US2004/0121370. The entire content of US2004/0121370 is incorporated herein by reference.

### TGF-β Antagonism

The role of TGF-β in tumorigenesis is complex. Clinical and mouse model data have shown that the TGF-β system can function as a tumor suppressor pathway, and therefore reduction or loss of TGF-β receptors or downstream signaling components is seen in many human tumors. However, late stage human tumors frequently show a paradoxical increase in expression of TGF-β that is associated with increased metastasis and poor prognosis. (See, for example, Neuzillet, C., et al. Pharmacology & Therapetuics, Vol. 147: 22-31, 2015; Ikushima, H., et al., Nature Reviews, Vol. 10: 415-424, 2010; Pickup, M., et al., Nature Reviews, Vol. 13: 788-799, 2013).

The TGF-β signaling pathway consists of three primary ligands (i.e., TGF-β1, TGF-β2, and TGF-β3) and two serine/threonine kinase receptors (i.e., TGF-β Receptor I (TGFβRI) and TGF-β Receptor II (TGFβRII)). The pathway also consists of a type 3 receptor (TGFβRIII; i.e., betaglycan) which has two TGF-β binding sites in its extracellular domain. TGFβRII is the specific receptor for all three TGF-β ligands. Binding of the ligand to TGFβRII causes formation of a heterotetrameric complex with TGFβRI, wherein TGFβRII phosphorylates TGFβRI. Phosphorylated TGFβRI recruits and phosphorylates receptor Smads (R-Smads), such as Smad2 and Smad3. These activated R-Smads form a complex with Smad4, which translocates to the nucleus to act as a transcription factor.

Several inhibitors of various components of the TGF-β pathway have been contemplated and developed (Korpal, M. and Kang Y., 2010 Eur J Cancer 46, 1232-1240; Bonafoux, D. and Wen-Cherng. L., 2009 Expert Opin Ther Patents 19, 17591769; Prudhomme, G. J., 2007 Laboratory Investigation 87, 1077-1091). In certain embodiments, inhibition of the TGF-β pathway is accomplished by blocking ligand synthesis, ligand-receptor interaction, or signal transduction. These antagonists fall into three major classes: (a) ligand traps, including monoclonal TGF-β-neutralizing antibodies and soluble receptors (including fusions and peptides thereof); (b) nucleic acid-based therapies, including antisense oligodeoxynucleotides (ODNs), ribozymes, small inhibitory RNA (siRNA), Smad6 or Smad7; and (c) small molecules that inhibit TGF-β signaling by, for example, blocking TGF-β1. Exemplary agents capable of inhibiting the TGF-β signaling pathway are disclosed in Published PCT Application WO 2015/140150, for example in Table 1, and U.S. Pat. No. 8,800,906 B2, the contents of each are herein incorporated by reference.

Although inhibition of the TGF-β pathway is a viable option for cancer therapeutics, there remains a need to identify combination therapies to further modulate the immune response in cancer patient.

In some aspects, the present disclosure provides a combination of a TGF-β antagonist and a CD155/TIGIT pathway antagonist to treat cancer. In certain embodiments, the TGF-β antagonist is an agent capable of inhibiting the TGF-β pathway. In other embodiments, the TGF-β antagonist is an agent capable of binding a component of the TGF-β pathway. In certain embodiments, the TGF-β antagonist binds TGF-β1.

### A. TGF-β Traps

In some embodiments, the TGF-β antagonist is a polypeptide. In some embodiments, the TGF-β antagonist is a peptide moiety that functionally neutralizes one or more isoforms of TGF-β. In certain embodiments, the peptide moiety is a TGF-β sequestering trap comprising an extracellular domain of a TGF-β receptor or an engineered polypeptide that specifically binds to and functionally neutralizes one or more TGF-β isoforms. In some embodiments, the TGF-β isoform neutralized is TGF-β1 since it is an important mediator of immune regulation and its association with cancer progression has been well documented. Therefore, the TGF-β antagonists described infra encompass antagonists that specifically target TGF-β1. In some embodiments, a TGF-β sequestering trap comprises the extracellular domain of the type 2 TGF-β receptor (TGFβRII) and functionally neutralizes one or more TGF-β isoforms. In some embodiments, the TGF-β antagonist is a soluble form of a TGF-β receptor protein. In some embodiments, the TGF-β receptor protein is an extracellular domain of the type 2 TGF-β receptor (TGFβRII) or a TGF-β1-binding fragment thereof. In some embodiments, the TGF-β receptor protein comprises the amino acid sequence set forth in SEQ ID NO: 5 (amino acids Thr23-Asp159), corresponding to the extracellular domain of TGFβRII.

In some embodiments, the TGF-β antagonist is a fusion protein comprising an extracellular domain of the type 2 TGF-β receptor, or a TGF-β1-binding fragment thereof, and a moiety that enhances the serum half-life of the TGF-β antagonist. In certain embodiments, the moiety that enhances the serum half-life of the TGF-β antagonist is a polyethylene glycol, an Fc portion of an antibody, or an albumin protein. In certain embodiments, the TGF-β antagonist is a fusion protein comprising an extracellular domain of the type 2 TGF-β receptor, or a TGF-β1-binding fragment thereof, and a human IgG1 Fc domain. In certain embodiments, the TGF-β antagonist comprises (a) an extracellular domain of the type 2 TGF-β receptor comprising the amino acid sequence depicted in SEQ ID NO: 5, and (b) a human IgG1 Fc domain comprising the amino acid sequence depicted in SEQ ID NO: 2.

In certain embodiments, the TGF-β antagonist is a bivalent TGF-β trap as described by Zwaagstra et al. (Mol Cancer Ther; 11(7): 1477-87, 2012). Specifically, traps using “natural sequence” flexible linkers by fusing the C-terminal disordered region from one receptor ectodomain to the N-terminal disordered region of the second ectodomain are used in the present disclosure.

In some embodiments, the TGF-β antagonist comprising the extracellular domain of the type 2 TGF-β receptor (or a TGF-β1-binding fragment thereof) is dimeric. Dimerization of such moieties has been shown to increase inhibitory potency by increased affinity through avidity. See, e.g., De Crescenzo et al. (2004) J Biol Chem 279(25):26013-26018, the disclosure of which is incorporated herein by reference in its entirety. One of skill in the art would appreciate the numerous ways in which such moieties can be multimerized, such as, but not limited to, fusions with a dimerization domain (e.g., an immunoglobulin Fc domain, as described above), post-expression cross-linking, and the like. A dimerization domain is an amino acid sequence capable of associating with or binding to another dimerization domain. The association or binding may be covalent or non-covalent. And there are two types of dimers, those capable of forming homodimers (with the same sequence), or heterodimers (with another sequence). In some embodiments, the dimerization domain is a leucine zipper coiled coil polypeptide. A leucine zipper typically comprises about 35 amino acids containing a characteristic seven residue repeat with hydrophobic residues at the first and fourth residues of the repeat (Harbury et al. (1993), Science 262:1401). Thus a leucine zipper is amenable to fusion to a polypeptide for the purpose of oligomerizing the polypeptide as it is a small molecule and is less likely to disrupt the polypeptides normal function than would a larger interaction domain. Examples of leucine zippers include but are not limited leucine zipper domains from polypeptides such as GCN4, C/EBP, c-Fos, c-Jun, c-Myc and c-Max.

Additional examples of dimerization domains include helix-loop-helix domains (Murre et al. (1989), Cell 58:537-544). The retinoic acid receptor, thyroid hormone receptor, other nuclear hormone receptors (Kurokawa et al. (1993), Genes Dev. 7:1423-1435) and yeast transcription factors GAL4 and HAP1 (Marmonstein et al. (1992), Nature 356:408-414; Zhang et al. (1993), Proc. Natl. Acad. Sci. USA 90:2851-2855; U.S. Pat. No. 5,624,818) all have dimerization domains with this motif.

In some embodiments, the association or binding between two proteins may be facilitated by the binding of non-amino acid molecules to the dimerization domain. For example, an avidin molecule attached to one dimerization domain and a biotin molecule attached to another dimerization domain would result in formation of an avidin-biotin bridge, thus promoting dimerization of the two domains.

Two proteins can be cross-linked using any of a number of known chemical cross linkers. Examples of such cross linkers are those which link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate). Preferably the cross-linking of two proteins will not interfere or significantly interfere with the function of the proteins (e.g., antagonist function). One of skill in the art is well aware of methods to evaluate the activity of cross-linked proteins, such as cross-linked TGF-β antagonists, including the protein binding studies described herein (e.g., TGF-β1: TGF-β Trap binding methods) and the functional studies exemplified in the working examples.

In some embodiments, the TGF-β antagonist is a heterotrimeric fusion trap comprising the ectodomain of TGFβRII coupled to the N- and C-terminal ends of the endoglin-domain of TGFβRIII, as described in Published US Patent Application 2015/0045299, herein incorporated by reference.

1. Serum Half-Life Enhancing Moiety

As described supra, in certain embodiments a TGF-β antagonist (e.g., TGFβRII extracellular domain) is fused to a moiety that enhances the serum half-life of the TGF-β antagonist. Non-limiting examples of serum half-life enhancing moieties are described infra. It should be understood that other moieties that increase the circulation half-life of the TGF-β antagonist (e.g., TGFβRII extracellular domain) are also applicable to the present disclosure. In certain embodiments, the serum half-life enhancing moiety is an antibody Fc domain (e.g., IgG1 Fc domain).

In certain embodiments, the serum half-life of a TGF-β antagonist (e.g., TGFβRII extracellular domain) fused to a serum half-life enhancing moiety is increased relative to a TGF-β antagonist (e.g., TGFβRII extracellular domain) alone (i.e., not fused to a serum half-life enhancing moiety). In certain embodiments, the serum half-life of a TGF-β antagonist (e.g., TGFβRII extracellular domain) fused to a serum half-life enhancing moiety is at least 20, 40, 60, 80, 100, 120, 150, 180, 200, 400, 600, 800 or 1000% longer relative to the serum half-life of TGF-β antagonist (e.g., TGFβRII extracellular domain) alone. In certain embodiments, the serum half-life of the TGF-β antagonist (e.g., TGFβRII extracellular domain) fused to a serum half-life enhancing moiety is at least 1.5- fold, 2-fold, 2.5-fold, 3-fold, 3.5 fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of the TGF-β antagonist (e.g., TGFβRII extracellular domain) alone. In certain embodiments, the serum half-life of the TGF-β antagonist (e.g., TGFβRII extracellular domain) fused to a serum half-life enhancing moiety is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

Fc Domains

In certain embodiments, a TGF-β antagonist (e.g., TGFβRII extracellular domain) includes an Fc domain, such as that with an amino acid sequences set forth in SEQ ID NO: 5. The Fc domain does not contain a variable region that binds to antigen. Fc domains useful for producing the TGF-β antagonist (e.g., TGFβRII extracellular domain) suitable for use in the methods disclosed herein may be obtained from a number of different sources. In certain embodiments, an Fc domain of the TGF-β antagonist (e.g., TGFβRII extracellular domain) is derived from a human immunoglobulin. In certain embodiments, the Fc domain is from a human IgG1 constant region (SEQ ID NO: 1). The Fc domain of human IgG1 is set forth in SEQ ID NO: 2. It is understood, however, that the Fc domain may be derived from an immunoglobulin of another mammalian species, including for example, a rodent (e.g. a mouse, rat, rabbit, guinea pig) or non- human primate (e.g. chimpanzee, macaque) species. Moreover, the Fc domain or portion thereof may be derived from any immunoglobulin class, including IgM, IgG, IgD, IgA, and IgE, and any immunoglobulin isotype, including IgG1, IgG2, IgG3, and IgG4.

In some aspects, a TGF-β antagonist (e.g., TGFβRII extracellular domain) includes a mutant Fc domain. In some aspects, a TGF-β antagonist (e.g., TGFβRII extracellular domain) includes a mutant, IgG1 Fc domain. In some aspects, a mutant Fc domain comprises one or more mutations in the hinge, CH2, and/or CH3 domains. In some aspects, a mutant Fc domain includes a D265A mutation.

In certain embodiments, an Fc domain employed in the TGF-β antagonist (e.g., TGFβRII extracellular domain) suitable for use in the methods disclosed herein is altered or modified, e.g., by amino acid mutation (e.g., addition, deletion, or substitution). As used herein, the term “Fc domain variant” refers to an Fc domain having at least one amino acid modification, such as an amino acid substitution, as compared to the wild-type Fc from which the Fc domain is derived. For example, wherein the Fc domain is derived from a human IgG1 antibody, a variant comprises at least one amino acid mutation (e.g., substitution) as compared to a wild type amino acid at the corresponding position of the human IgG1 Fc region. Exemplary mutations are disclosed in Published U.S. Patent Application Nos. 2006/0235208, 2003/0108548, US 2007/0111281; U.S. Pat. No. 5,624,821; and International PCT Publication Nos. WO 2005/063815, WO 2005/018572, each document is hereby incorporated by reference.

PEGylation

In certain embodiments, a TGF-β antagonist (e.g., TGFβRII extracellular domain) suitable for use in the methods disclosed herein includes a polyethylene glycol (PEG) domain. PEGylation is well known in the art to confer increased circulation half-life to proteins. Methods of PEGylation are well known and disclosed in, e.g., U.S. Pat. No. 7,610,156, U.S. Pat. No. 7,847,062, all of which are hereby incorporated by reference.

PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ₋₁CH₂CH₂OH, where n is 20 to 2300 and X is H or a terminal modification, e.g., a C₁₋₄ alkyl. In certain embodiments, the PEG suitable for use in the methods disclosed herein terminates on one end with hydroxy or methoxy, i.e., X is H or CH₃ (“methoxy PEG”). PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP- A 0 473 084 and U.S. Pat. No. 5,932,462, both of which are hereby incorporated by reference. One form of PEGs includes two PEG side-chains (PEG2) linked via the primary amino groups of a lysine (Monfardini et al., Bioconjugate Chem 1995;6:62-9).

Other Serum Half-Life Enhancing Moieties

In certain embodiments, the serum half-life enhancing moiety is a serum albumin, or fragments thereof. Methods of fusing serum albumin to proteins are disclosed in, e.g., US2010/0144599, US2007/0048282, and US2011/0020345, which are herein incorporated by reference in their entirety. In certain embodiments, the serum half-life enhancing moiety is human serum albumin (HSA), or variants or fragments thereof, such as those disclosed in U.S. Pat. No. 5,876,969, WO 2011/124718, WO 2013/075066, and WO 2011/0514789.

In certain embodiments, the serum half-life enhancing moiety is a serum albumin binding protein such as those described in US2005/0287153, US2007/0003549, US2007/0178082, US2007/0269422, US2010/0113339, WO2009/083804, and WO2009/133208, which are herein incorporated by reference in their entirety.

In certain embodiments, the serum half-life enhancing moiety is transferrin, as disclosed in U.S. Pat. No. 7,176,278 and U.S. Pat. No. 8,158,579, which are herein incorporated by reference in their entirety.

In certain embodiments, the serum half-life enhancing moiety is a serum immunoglobulin binding protein such as those disclosed in US2007/0178082, which is herein incorporated by reference in its entirety.

In certain embodiments, the serum half-life enhancing moiety is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3(HSA), i.e., a Fn3 protein that binds to human serum albumin.

2. Methods of Making TGF-β Traps

Also provided in the disclosure are methods for generating the TGF-β antagonist molecules for use in the methods described herein. In certain embodiments, the TGF-β antagonist is a fusion protein comprising an extracellular domain of a TGF-β receptor and a moiety that enhances the serum half-life of the antagonist.

Fc Domain

In certain embodiments, the moiety that enhances the serum half-life of the TGF-β antagonist (e.g., TGFβRII extracellular domain) is an Fc domain. A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or portions thereof) can be derived from these sequences using art recognized techniques. The genetic material obtained using any of the foregoing methods may then be altered or synthesized to obtain polypeptides suitable for use in the methods disclosed herein. It will further be appreciated that the scope of this invention encompasses alleles, variants and mutations of constant region DNA sequences.

Fc domain sequences can be cloned, e.g., using the polymerase chain reaction and primers which are selected to amplify the domain of interest. To clone an Fc domain sequence from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, Calif. (1990); Ho et al. 1989. Gene 77:51; Horton et al. 1993. Methods Enzymol. 217:270). PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7: 1509); rapid amplification of cDNA ends (Ruberti, F. et al. 1994. J. Immunol. Methods 173:33); antibody leader sequences (Larrick et al. Biochem Biophys Res Commun 1989; 160: 1250). The cloning of antibody sequences is further described in Newman et al., U.S. Pat. No. 5,658,570, filed Jan. 25, 1995, which is herein incorporated by reference.

TGF-β antagonists (e.g., TGFβRII extracellular domain) suitable for use in the methods disclosed herein may comprise one or more Fc domains (e.g., 2, 3, 4, 5, 6, 7, 8, 9, 10, or more Fc domains). In certain embodiments, the Fc domains may be of different types. In certain embodiments, at least one Fc domain present in the TGF-β antagonist (e.g., TGFβRII extracellular domain) comprises a hinge domain or portion thereof. In certain embodiments, the TGF-β antagonist (e.g., TGFβRII extracellular domain) suitable for use in the methods disclosed herein comprises at least one Fc domain which comprises at least one CH2 domain or portion thereof. In certain embodiments, the TGF-β antagonist (e.g., TGFβRII extracellular domain) suitable for use in the methods disclosed herein comprises at least one Fc domain which comprises at least one CH3 domain or portion thereof. In certain embodiments, the TGF-β antagonist (e.g., TGFβRII extracellular domain) suitable for use in the methods disclosed herein comprises at least one Fc domain which comprises at least one CH4 domain or portion thereof. In certain embodiments, the TGF-β antagonist (e.g., TGFβRII extracellular domain) suitable for use in the methods disclosed herein comprises at least one Fc domain which comprises at least one hinge domain or portion thereof and at least one CH2 domain or portion thereof (e.g., in the hinge-CH2 orientation). In certain embodiments, the TGF-β antagonist (e.g., TGFβRII extracellular domain) suitable for use in the methods disclosed herein comprises at least one Fc domain which comprises at least one CH2 domain or portion thereof and at least one CH3 domain or portion thereof (e.g., in the CH2-CH3 orientation). In certain embodiments, the TGF-β antagonist (e.g., TGFβRII extracellular domain) suitable for use in the methods disclosed herein comprises at least one Fc domain comprising at least one hinge domain or portion thereof, at least one CH2 domain or portion thereof, and least one CH3 domain or portion thereof, for example in the orientation hinge-CH2-CH3, hinge-CH3-CH2, or CH2-CH3-hinge.

In certain embodiments, a TGF-β antagonist (e.g., TGFβRII extracellular domain) comprises at least one complete Fc region derived from one or more immunoglobulin heavy chains (e.g., an Fc domain including hinge, CH2, and CH3 domains, although these need not be derived from the same antibody). In certain embodiments, a TGF-β antagonist (e.g., TGFβRII extracellular domain) comprises at least two complete Fc domains derived from one or more immunoglobulin heavy chains. In certain embodiments, the complete Fc domain is derived from a human IgG immunoglobulin heavy chain (e.g., human IgG1).

In certain embodiments, the TGF-β antagonist (e.g., TGFβRII extracellular domain) suitable for use in the methods disclosed herein comprises at least one Fc domain comprising a complete CH3 domain. In certain embodiments, the TGF-β antagonist (e.g., TGFβRII extracellular domain) suitable for use in the methods disclosed herein comprises at least one Fc domain comprising a complete CH2 domain. In certain embodiments, the TGF-β antagonist (e.g., TGFβRII extracellular domain) suitable for use in the methods disclosed herein comprises at least one Fc domain comprising at least a CH3 domain, and at least one of a hinge region, and a CH2 domain. In certain embodiments, the TGF-β antagonist (e.g., TGFβRII extracellular domain) suitable for use in the methods disclosed herein comprises at least one Fc domain comprising a hinge and a CH3 domain. In certain embodiments, the TGF-β antagonist (e.g., TGFβRII extracellular domain) suitable for use in the methods disclosed herein comprises at least one Fc domain comprising a hinge, a CH2, and a CH3 domain. In certain embodiments, the Fc domain is derived from a human IgG immunoglobulin heavy chain (e.g., human IgG1).

The constant region domains or portions thereof making up an Fc domain of the TGF-β antagonist (e.g., TGFβRII extracellular domain) suitable for use in the methods disclosed herein may be derived from different immunoglobulin molecules. For example, a polypeptide suitable for use in the methods disclosed herein may comprise a CH2 domain or portion thereof derived from an IgG1 molecule and a CH3 region or portion thereof derived from an IgG3 molecule. In another example, the TGF-β antagonist (e.g., TGFβRII extracellular domain) can comprise an Fc domain comprising a hinge domain derived, in part, from an IgG1 molecule and, in part, from an IgG3 molecule. As set forth herein, it will be understood by one of ordinary skill in the art that an Fc domain may be altered such that it varies in amino acid sequence from a naturally occurring antibody molecule.

In certain embodiments, the TGF-β antagonist (e.g., TGFβRII extracellular domain) suitable for use in the methods disclosed herein lacks one or more constant region domains of a complete Fc region, i.e., they are partially or entirely deleted. In certain embodiments, the TGF-β antagonist (e.g., TGFβRII extracellular domain) suitable for use in the methods disclosed herein will lack an entire CH2 domain. In certain embodiments, the TGF-β antagonist (e.g., TGFβRII extracellular domain) suitable for use in the methods disclosed herein comprise CH2 domain-deleted Fc regions derived from a vector (e.g., from IDEC Pharmaceuticals, San Diego) encoding an IgG1 human constant region domain (see, e.g., WO02/060955A2 and WO02/096948A2). This exemplary vector is engineered to delete the CH2 domain and provide a synthetic vector expressing a domain-deleted IgG1 constant region. It will be noted that these exemplary constructs are preferably engineered to fuse a binding CH3 domain directly to a hinge region of the respective Fc domain.

In other constructs it may be desirable to provide a peptide spacer between one or more constituent Fc domains. For example, a peptide spacer may be placed between a hinge region and a CH2 domain and/or between a CH2 and a CH3 domain. For example, compatible constructs could be expressed wherein the CH2 domain has been deleted and the remaining CH3 domain (synthetic or unsynthetic) is joined to the hinge region with a 1-20, 1-10, or 1-5 amino acid peptide spacer. Such a peptide spacer may be added, for instance, to ensure that the regulatory elements of the constant region domain remain free and accessible or that the hinge region remains flexible. Preferably, any linker peptide compatible used in the instant invention will be relatively non-immunogenic and not prevent proper folding of the Fc.

PEGylation

In certain embodiments, pegylated TGF-β antagonist (e.g., TGFβRII extracellular domain) is produced by site-directed pegylation, particularly by conjugation of PEG to a cysteine moiety at the N- or C-terminus. A PEG moiety may also be attached by other chemistry, including by conjugation to amines. PEG conjugation to peptides or proteins generally involves the activation of PEG and coupling of the activated PEG-intermediates directly to target proteins/peptides or to a linker, which is subsequently activated and coupled to target proteins/peptides (see Abuchowski et al., JBC 1977;252:3571 and JBC 1977;252:3582, and Harris et. al., in: Poly(ethylene glycol) Chemistry: Biotechnical and Biomedical Applications; (J. M. Harris ed.) Plenum Press: New York, 1992; Chap. 21 and 22). A variety of molecular mass forms of PEG can be selected, e.g., from about 1,000 Daltons (Da) to 100,000 Da (n is 20 to 2300), for conjugating to a TGF-β antagonist (e.g., TGFβRII extracellular domain). The number of repeating units “n” in the PEG is approximated for the molecular mass described in Daltons. It is preferred that the combined molecular mass of PEG on an activated linker is suitable for pharmaceutical use. Thus, in one embodiment, the molecular mass of the PEG molecules does not exceed 100,000 Da. For example, if three PEG molecules are attached to a linker, where each PEG molecule has the same molecular mass of 12,000 Da (each n is about 270), then the total molecular mass of PEG on the linker is about 36,000 Da (total n is about 820). The molecular masses of the PEG attached to the linker can also be different, e.g., of three molecules on a linker two PEG molecules can be 5,000 Da each (each n is about 110) and one PEG molecule can be 12,000 Da (n is about 270).

One skilled in the art can select a suitable molecular mass for PEG, e.g., based on how the pegylated TGF-β antagonist (e.g., TGFβRII extracellular domain) will be used therapeutically, the desired dosage, circulation time, resistance to proteolysis, immunogenicity, and other considerations. For a discussion of PEG and its use to enhance the properties of proteins, see N. V. Katre, Advanced Drug Delivery Reviews 1993;10:91-114.

In certain embodiments, carbonate esters of PEG are used to form the PEG-TGF-β antagonist (e.g., TGFβRII extracellular domain) conjugates. N,N′-disuccinimidylcarbonate (DSC) may be used in the reaction with PEG to form active mixed PEG-succinimidyl carbonate that may be subsequently reacted with a nucleophilic group of a linker or an amino group of TGF-β antagonist (e.g., TGFβRII extracellular domain) (see U.S. Pat. No. 5,281,698 and U.S. Pat. No. 5,932,462). In a similar type of reaction, 1,1′-(dibenzotriazolyl)carbonate and di-(2-pyridyl)carbonate may be reacted with PEG to form PEG-benzotriazolyl and PEG-pyridyl mixed carbonate (U.S. Pat. No. 5,382,657), respectively. Pegylation of a TGF-β antagonist (e.g., TGFβRII extracellular domain) can be performed according to the methods of the state of the art, for example by reaction of a TGF-β antagonist (e.g., TGFβRII extracellular domain) with electrophilically active PEGs (Shearwater Corp., USA, www.shearwatercorp.com). Preferred PEG reagents suitable for use in the methods disclosed herein are, e.g., N-hydroxysuccinimidyl propionates (PEG-SPA), butanoates (PEG-SBA), PEG-succinimidyl propionate or branched N-hydroxysuccinimides such as mPEG2-NHS (Monfardini, C, et al., Bioconjugate Chem. 6 (1995) 62-69).

In certain embodiments, PEG molecules may be coupled to sulfhydryl groups on IL-2 (Sartore, L., et al., Appl. Biochem. Biotechnol., 27, 45 (1991); Morpurgo et al., Biocon. Chem., 7, 363-368 (1996); Goodson et al., Bio/Technology (1990) 8, 343; U.S. Pat. No. 5,766,897). U.S. Pat. No. 6,610,281 and U.S. Pat. No. 5,766,897 describe exemplary reactive PEG species that may be coupled to sulfhydryl groups.

In certain embodiments where PEG molecules are conjugated to cysteine residues on a TGF-β antagonist (e.g., TGFβRII extracellular domain) the cysteine residues are native to a TGF-β antagonist (e.g., TGFβRII extracellular domain) whereas in certain embodiments, one or more cysteine residues are engineered into a TGF-β antagonist (e.g., TGFβRII extracellular domain). Mutations may be introduced into the coding sequence of a TGF-β antagonist (e.g., TGFβRII extracellular domain) to generate cysteine residues. This might be achieved, for example, by mutating one or more amino acid residues to cysteine. Preferred amino acids for mutating to a cysteine residue include serine, threonine, alanine and other hydrophilic residues. Preferably, the residue to be mutated to cysteine is a surface-exposed residue. Algorithms are well-known in the art for predicting surface accessibility of residues based on primary sequence or a protein.

In certain embodiments, pegylated TGF-β antagonist (e.g., TGFβRII extracellular domain) comprise one or more PEG molecules covalently attached to a linker.

In certain embodiments, a TGF-β antagonist (e.g., TGFβRII extracellular domain) is pegylated at the C-terminus. In certain embodiments, a protein is pegylated at the C-terminus by the introduction of C-terminal azido-methionine and the subsequent conjugation of a methyl-PEG-triarylphosphine compound via the Staudinger reaction. This C-terminal conjugation method is described in Cazalis et al., C-Terminal Site-Specific PEGylation of a Truncated Thrombomodulin Mutant with Retention of Full Bioactivity, Bioconjug Chem. 2004; 15(5): 1005- 1009. Monopegylation of a TGF-β antagonist (e.g., TGFβRII extracellular domain) can also be achieved according to the general methods described in WO 94/01451. WO 94/01451 describes a method for preparing a recombinant polypeptide with a modified terminal amino acid alpha-carbon reactive group. The steps of the method involve forming the recombinant polypeptide and protecting it with one or more biologically added protecting groups at the N-terminal alpha-amine and C-terminal alpha-carboxyl. The polypeptide can then be reacted with chemical protecting agents to selectively protect reactive side chain groups and thereby prevent side chain groups from being modified. The polypeptide is then cleaved with a cleavage reagent specific for the biological protecting group to form an unprotected terminal amino acid alpha-carbon reactive group. The unprotected terminal amino acid alpha-carbon reactive group is modified with a chemical modifying agent. The side chain protected terminally modified single copy polypeptide is then deprotected at the side chain groups to form a terminally modified recombinant single copy polypeptide. The number and sequence of steps in the method can be varied to achieve selective modification at the N- and/or C-terminal amino acid of the polypeptide.

The ratio of TGF-β antagonist (e.g., TGFβRII extracellular domain) to activated PEG in the conjugation reaction can be from about 1:0.5 to 1:50, between from about 1: 1 to 1:30, or from about 1:5 to 1:15. Various aqueous buffers can be used to catalyze the covalent addition of PEG to a TGF-β antagonist (e.g., TGFβRII extracellular domain). In certain embodiments, the pH of a buffer used is from about 7.0 to 9.0. In certain embodiments, the pH is in a slightly basic range, e.g., from about 7.5 to 8.5. Buffers having a pKa close to neutral pH range may be used, e.g., phosphate buffer.

Conventional separation and purification techniques known in the art can be used to purify PEGylated TGF-β antagonist (e.g., TGFβRII extracellular domain), such as size exclusion (e.g. gel filtration) and ion exchange chromatography. Products may also be separated using SDS-PAGE. Products that may be separated include mono-, di-, tri- poly- and un-pegylated IL-2 as well as free PEG. The percentage of mono-PEG conjugates can be controlled by pooling broader fractions around the elution peak to increase the percentage of mono-PEG in the composition.

In certain embodiments, PEGylated TGF-β antagonist (e.g., TGFβRII extracellular domain) suitable for use in the methods disclosed herein contains one, two or more PEG moieties. In certain embodiments, the combined or total molecular mass of PEG in PEG-TGF-β antagonist (e.g., TGFβRII extracellular domain) is from about 3,000 Da to 60,000 Da, optionally from about 10,000 Da to 36,000 Da. In certain embodiments, PEG in pegylated TGF-β antagonist (e.g., TGFβRII extracellular domain) is a substantially linear, straight-chain PEG.

In certain embodiments, pegylated TGF-β antagonist (e.g., TGFβRII extracellular domain) suitable for use in the methods disclosed herein will preferably retain at least 25%, 50%, 60%, 70%, 80%, 85%, 90%, 95% or 100% of the biological activity associated with the unmodified protein. In certain embodiments, biological activity refers to the ability to bind TGF-β1. The serum clearance rate of PEG-modified TGF-β antagonist (e.g., TGFβRII extracellular domain) may be decreased by about 10%, 20%, 30%, 40%, 50%, 60%, 70%, 80%, or even 90%, relative to the clearance rate of the unmodified TGF-β antagonist (e.g., TGFβRII extracellular domain). PEG-modified TGF-β antagonist (e.g., TGFβRII extracellular domain) may have a circulation half-life (t̂) which is enhanced relative to the half-life of unmodified TGF-β antagonist (e.g., TGFβRII extracellular domain). The half-life of PEG-TGF-β antagonist (e.g., TGFβRII extracellular domain), may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of unmodified TGF-β antagonist (e.g., TGFβRII extracellular domain). In certain embodiments, the protein half-life is determined in vitro, such as in a buffered saline solution or in serum. In certain embodiments, the protein half-life is an in vivo circulation half-life, such as the half-life of the protein in the serum or other bodily fluid of an animal.

Linkers

In certain embodiments, the serum half-life enhancing moiety is optionally fused to TGF-β antagonist (e.g., TGFβRII extracellular domain) via a linker. Linkers suitable for fusing the serum half-life enhancing moiety to TGF-β antagonist (e.g., TGFβRII extracellular domain) are well known in the art, and are disclosed in, e.g., US2010/0210511 US2010/0179094, and US2012/0094909, which are herein incorporated by reference in its entirety. Exemplary linkers include gly-ser polypeptide linkers, glycine-proline polypeptide linkers, and proline-alanine polypeptide linkers. In certain embodiments, the linker is a gly-ser polypeptide linker, i.e., a peptide that consists of glycine and serine residues.

Exemplary gly-ser polypeptide linkers comprise the amino acid sequence Ser(Gly₄Ser)n. In certain embodiments, n=1. In certain embodiments, n=2. In certain embodiments, n=3, i.e., Ser(Gly₄Ser)3. In certain embodiments, n=4, i.e., Ser(Gly₄Ser)4. In certain embodiments, n=5. In certain embodiments, n=6. In certain embodiments, n=7. In certain embodiments, n=8. In certain embodiments, n=9. In certain embodiments, n=10. Another exemplary gly-ser polypeptide linker comprises the amino acid sequence Ser(Gly₄Ser)n. In certain embodiments, n=1. In certain embodiments, n=2. In certain embodiments, n=3. In certain embodiments, n=4. In certain embodiments, n=5. certain embodiments, n=6. Another exemplary gly-ser polypeptide linker comprises (Gly₄Ser)n. In certain embodiments, n=1. In certain embodiments, n=2. In certain embodiments, n=3. In certain embodiments, n=4. In certain embodiments, n=5. In certain embodiments, n=6. Another exemplary gly-ser polypeptide linker comprises (Gly₃Ser)n. In certain embodiments, n=1. In certain embodiments, n=2. In certain embodiments, n=3. In certain embodiments, n=4. In certain embodiments, n=5. In certain embodiments n=6.

### B. Other TGF-β Antagonists

In some embodiments, the TGF-β antagonist is a small molecule inhibitor. In certain embodiments, a small molecule inhibitor targets the TGF-β receptor type I kinase (ALK5). In certain embodiments, the antagonist is an ALK5 inhibitor. In certain embodiments the inhibitor of TGF-β is SB431542 (see. E.g. Laping, N.J., et. al., (2002), Molecular Pharmacology 62 (1): 58-64, commercially available e.g. Sigma Prod. No. S 4317). In certain embodiments, a small molecule inhibitor minimizes ligand-receptor interactions by directly blocking the catalytic activity of the TGF-β receptor kinase. Several small molecule inhibitors have been shown to have a preclinical effect, such as SD-208 (small molecule inhibitor of the ATP binding site of the TGF-βRI kinase; Bonniaud, P. et al., 2005 Am J Respir Crit. Care Med. 171, 889-98; Uhl, M. et al., 2004 Cancer Res 64, 7954-61; Ge, R. et al., 2006 Clin Cancer Res 12, 4315-30)and Ki26894 (small molecule TGF-13RI kinase inhibitor; Ehata, S. et al., 2007 Cancer Sci 98, 127-33). In certain embodiments, a small molecule inhibitor targets both TGF-β receptors. For example, LY2109761 is a small molecule TGF-βRI and TGF-βRII dual inhibitor shown to have a preclinical effect (Melisi, D. et al., 2008 Mol Cancer Therapy 7, 829-40; Zhang, B. et al,, 2009 Cancer Lett 277, 114-20). In addition, LY2157299 is a small molecule TGF-βRI kinase inhibitor entering Phase I clinical trials (Calvo-Aller, E. B. J. et al., 2008 J Clin Oncol 26 Abstract # 14554).

In some embodiments, a small molecule inhibitor targets downstream signaling effectors of the TGF-β pathway. For example, the small molecule SIS3 (6,7-dimethyl-2-[(2E)-3-(1-methyl-2-phenyl-1H-pyrrolo[2,3-b]pyridin-3-yl-prop-2-enoyl)-1,2,3,4-tetrahydroisoquinoline hydrochloride) was described by Jinnin et al., (Jinnin M. et al., 2006, Mol Pharmacol 69: 597-607) as a specific inhibitor of Smad3 that suppresses Smad3 phosphorylation, DNA-Smad3 binding, and the interaction of Smad3 with Smad4.

In some embodiments, a TGF-β antagonist is a neutralizing anti-TGF-β antibody or binding fragment thereof. In some embodiments, the anti-TGF-β antibody neutralizes a TGF-β ligand (i.e., TGF-β1, TGF-(32, or TGF-β3). In some embodiments, the anti-TGF-β antibody neutralizes a TGF-β receptor (i.e., TGF-β receptor type 1 or type 2). Neutralizing antibodies minimize interactions between ligands and receptors. In certain embodiments, the neutralizing ability of an antibody is determined using a neutralization assay. An example of a neutralization assay is determining the neutralization of the growth inhibitory activity of TGF-β1, TGF-β2, and TGF-β3 in vitro on mink lung MvILu epithelial cells, as described in Lucas, C. et al 1990 J. Immunol. 145, 1415-1472.

In some embodiments, a TGF-β antagonist is an antisense oligonucleotide. Antisense oligonucleotides are single-stranded polynucleotide molecules, usually 13-25 nucleotides in length that hybridize to complementary RNA, inhibit mRNA function, and prevent protein synthesis through accelerated mRNA degradation by RNase H or steric blockade. In certain embodiments, the antisense oligonucleotide is specific for mRNA encoding TGF-β1, TGF-β2, or TGF-β3 isotypes or other components of the TGF-β signaling assembly. In certain embodiments, the antisense oligonucleotide comprises a modified nucleoside such as 2′-O, 4′-c-methylene linked bicyclic ribonucleotides, known as locked nucleic acids (LNA; e.g., oxyl-LNA, amino-LNA, thio-LNA), phosphorodiamidate morpholino oligomers (PMO), phosphorothioate (PS), 2′-O-mehyl (2′-Ome), 2′-fluro (2′-fluoro (2′-F)), or 2′-methoxyethyl (2′-MOE) derivatives. Antisense-mediated inhibition of TGF-β1 gene expression has been shown to be effective in preclinical trials, with AP12009 being at an advanced stage of clinical development (Schlingensiepen, K. H. et al., 2008 Recent Results Cancer Res 177, 137-50). In some embodiments, a TGF-β antagonist is an antisense RNA molecule specific for TGF-β2 mRNA, such as belagenpumatucel-L, and/or TGF-β1 mRNA or TGF-β3 mRNA, or other components of mRNA encoding TGF-β signaling assembly.

In some embodiments, a TGF-β antagonist is a silencing RNA molecule (siRNA). siRNA refers to nucleotides of 19-23 bases in length which incorporate into an RNA-induced silencing complex in order to guide the complex to homologous endogenous mRNA for cleavage and degradation of the target mRNA. In some embodiments, a TGF-β antagonist is a silencing RNA molecule (siRNA) specifically for mRNA encoding TGF-β1, TGF-β2, or TGF-β3 isotypes or other components of the TGF-β signaling assembly.

In some embodiments, a TGF-β antagonist is a short hairpin RNA (shRNA). shRNA refers to an artificial double-stranded oligonucleotide with a tight hairpin turn that can be used to silence target gene expression via RNA interference. In some embodiments, a TGF-β antagonist is a short hairpin RNA (shRNA) specific for mRNA encoding TGF-β1, TGF-β2, or TGF-β3 isotypes or other components of the TGF-β signaling assembly.

In some embodiments, a TGF-β antagonist is a microRNA (miRNA) molecule microRNAs are small non-coding RNAs belonging to a class of regulatory molecules found in plants and animals that control gene expression by binding to complementary sites on target mRNA transcripts. In some embodiments, a TGF-β antagonist is a microRNA (miRNA) molecule specific for mRNA encoding TGF-β1, TGF-β2, or TGF-β3 isotypes or other components of the TGF-β signaling assembly

In some embodiments, a TGF-β antagonist is an aptamer and/or spiegelmer molecule. Aptamers are small peptide, DNA or RNA molecules containing a target-binding and a scaffolding domain that stabilize and interfere with the function of the target. Spiegelmers are L-RNA aptamers. In certain embodiments, TGF-β antagonist is an aptamer and/or spiegelmer molecule specific for TGF-β1, TGF-β2, or TGF-β3 isotypes or other components of the TGF-β signaling assembly.

In some embodiments, a TGF-β antagonist is a ribozyme. A ribozyme is an enzymatic nucleic acid (e.g., RNA) molecule that can cleave and/or ligate to a part of itself or to other separate nucleic acid molecules, and therefore down regulate expression of targeted RNA. In certain embodiments, the ribozyme is specific for mRNA encoding TGF-β1, TGF-β2, or TGF-β3 isotypes or other components of the TGF-β signaling assembly.

### Methods of Making Antibodies

In certain embodiments, the CD155/TIGIT antagonist and TGF-β1 antagonist, for use in the methods disclosed herein are antibodies or binding fragments thereof. In certain embodiments, the CD155/TIGIT antagonist is an anti-CD155 antibody. In certain embodiments, the CD155/TIGIT antagonist is an anti-TIGIT antibody. In certain embodiments, the TGF-β1 antagonist is an anti-TGF-β1 antibody. In certain embodiments, the TGF-β1 antagonist is an antibody that binds to a signaling component of the TGF-β pathway. The disclosure also features methods for producing any of the antibodies or antigen-binding fragments thereof described herein.

Also, encompassed by the present disclosure are antibodies that bind to an epitope on CD155, TIGIT or TGF-β pathway component (e.g., TGF-β1), which comprises all or a portion of an epitope recognized by the particular antibodies described herein (e.g., the same or an overlapping region or a region between or spanning the region).

Also encompassed by the present disclosure are antibodies that bind the same epitope and/or antibodies that compete for binding to human CD155, TIGIT or TGF-β pathway component (e.g., TGF-β1) with the antibodies described herein. Antibodies that recognize the same epitope or compete for binding can be identified using routine techniques. Such techniques include, for example, an immunoassay, which shows the ability of one antibody to block the binding of another antibody to a target antigen, i.e., a competitive binding assay. Competitive binding is determined in an assay in which the immunoglobulin under test inhibits specific binding of a reference antibody to a common antigen, such as CD155, TIGIT or TGF-β1. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

Other techniques include, for example, epitope mapping methods, such as, x-ray analyses of crystals of antigen:antibody complexes which provides atomic resolution of the epitope. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

In some embodiments, methods for preparing an antibody described herein can include immunizing a subject (e.g., a non-human mammal) with an appropriate immunogen. Suitable immunogens for generating any of the antibodies described herein are set forth herein. For example, to generate an antibody that binds to CD155, TIGIT or TGF-β pathway component (e.g., TGF-β1), a skilled artisan can immunize a suitable subject (e.g., a non-human mammal such as a rat, a mouse, a gerbil, a hamster, a dog, a cat, a pig, a goat, a horse, or a non-human primate) with a full-length polypeptide.

A suitable subject (e.g., a non-human mammal) can be immunized with the appropriate antigen along with subsequent booster immunizations a number of times sufficient to elicit the production of an antibody by the mammal. The immunogen can be administered to a subject (e.g., a non-human mammal) with an adjuvant. Adjuvants useful in producing an antibody in a subject include, but are not limited to, protein adjuvants; bacterial adjuvants, e.g., whole bacteria (BCG, Corynebacterium parvum or Salmonella minnesota) and bacterial components including cell wall skeleton, trehalose dimycolate, monophosphoryl lipid A, methanol extractable residue (MER) of tubercle bacillus, complete or incomplete Freund's adjuvant; viral adjuvants; chemical adjuvants, e.g., aluminum hydroxide, and iodoacetate and cholesteryl hemisuccinate. Other adjuvants that can be used in the methods for inducing an immune response include, e.g., cholera toxin and parapoxvirus proteins. See also Bieg et al. (1999) Autoimmunity 31(1):15-24. See also, e.g., Lodmell et al. (2000) Vaccine 18:1059-1066; Johnson et al. (1999) J Med Chem 42:4640-4649; Baldridge et al. (1999) Methods 19:103-107; and Gupta et al. (1995) Vaccine 13(14): 1263-1276.

In some embodiments, the methods include preparing a hybridoma cell line that secretes a monoclonal antibody that binds to the immunogen. For example, a suitable mammal such as a laboratory mouse is immunized with a polypeptide as described above (e.g., CD155, TIGIT or TGF-β1). Antibody-producing cells (e.g., B cells of the spleen) of the immunized mammal can be isolated two to four days after at least one booster immunization of the immunogen and then grown briefly in culture before fusion with cells of a suitable myeloma cell line. The cells can be fused in the presence of a fusion promoter such as, e.g., vaccinia virus or polyethylene glycol. The hybrid cells obtained in the fusion are cloned, and cell clones secreting the desired antibodies are selected. For example, spleen cells of Balb/c mice immunized with a suitable immunogen can be fused with cells of the myeloma cell line PAI or the myeloma cell line Sp2/0-Ag 14. After the fusion, the cells are expanded in suitable culture medium, which is supplemented with a selection medium, for example HAT medium, at regular intervals in order to prevent normal myeloma cells from overgrowing the desired hybridoma cells. The obtained hybrid cells are then screened for secretion of the desired antibodies, e.g., an antibody that binds to TIGIT and inhibits the interaction between TIGIT and a TIGIT receptor (e.g., CD155).

In some embodiments, a skilled artisan can identify an antibody from a non-immune biased library as described in, e.g., U.S. Pat. No. 6,300,064 (to Knappik et al.; Morphosys AG) and Schoonbroodt et al. (2005) Nucleic Acids Res 33(9):e81.

In some embodiments, the methods described herein can involve, or be used in conjunction with, e.g., phage display technologies, bacterial display, yeast surface display, eukaryotic viral display, mammalian cell display, and cell-free (e.g., ribosomal display) antibody screening techniques (see, e.g., Etz et al. (2001) J Bacteriol 183:6924-6935; Cornelis (2000) Curr Opin Biotechnol 11:450-454; Klemm et al. (2000) Microbiology 146:3025-3032; Kieke et al. (1997) Protein Eng 10:1303-1310; Yeung et al. (2002) Biotechnol Prog 18:212-220; Boder et al. (2000) Methods Enzymology 328:430-444; Grabherr et al. (2001) Comb Chem High Throughput Screen 4:185-192; Michael et al. (1995) Gene Ther 2:660-668; Pereboev et al. (2001) J Virol 75:7107-7113; Schaffitzel et al. (1999) J Immunol Methods 231:119-135; and Hanes et al. (2000) Nat Biotechnol 18:1287-1292).

In some embodiments, a combination of selection and screening can be employed to identify an antibody of interest from, e.g., a population of hybridoma-derived antibodies or a phage display antibody library. Suitable methods are known in the art and are described in, e.g., Hoogenboom (1997) Trends in Biotechnology 15:62-70; Brinkman et al. (1995), supra; Ames et al. (1995), supra; Kettleborough et al. (1994), supra; Persic et al. (1997), supra; and Burton et al. (1994), supra. For example, a plurality of phagemid vectors, each encoding a fusion protein of a bacteriophage coat protein (e.g., pIII, pVIII, or pIX of M13 phage) and a different antigen-combining region are produced using standard molecular biology techniques and then introduced into a population of bacteria (e.g., E. coli). Expression of the bacteriophage in bacteria can, in some embodiments, require use of a helper phage. In some embodiments, no helper phage is required (see, e.g., Chasteen et al. (2006) Nucleic Acids Res 34(21):e145). Phage produced from the bacteria are recovered and then contacted to, e.g., a target antigen bound to a solid support (immobilized). Phage may also be contacted to antigen in solution, and the complex is subsequently bound to a solid support.

A subpopulation of antibodies screened using the above methods can be characterized for their specificity and binding affinity for a particular antigen (e.g., CD155, TIGIT or TGF-β1) using any immunological or biochemical based method known in the art. For example, specific binding of an antibody, may be determined for example using immunological or biochemical based methods such as, but not limited to, an ELISA assay, SPR assays, immunoprecipitation assay, affinity chromatography, and equilibrium dialysis as described above. Immunoassays which can be used to analyze immunospecific binding and cross-reactivity of the antibodies include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, and protein A immunoassays. Such assays are routine and well known in the art.

The antibodies or antigen-binding fragments thereof described herein can be produced using a variety of techniques known in the art of molecular biology and protein chemistry. For example, a nucleic acid encoding one or both of the heavy and light chain polypeptides of an antibody can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Appropriate host cells for the expression of antibodies or antigen-binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof can be expressed in, and purified from, transgenic animals (e.g., transgenic mammals). For example, an antibody can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

An antibody or fragment thereof can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) “Protein Purification, 3^{rd} edition,” Springer-Verlag, New York City, N.Y. The degree of purification necessary will vary depending on the desired use. In some instances, no purification of the expressed antibody or fragments thereof will be necessary.

### Methods of Making Polypeptides

In some aspects, the polypeptides described herein (e.g., CD155/TIGIT antagonist and/or a TGF-β1 antagonist) are made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The methods of making polypeptides also include a vector capable of expressing the peptides in an appropriate host. The vector comprises the DNA molecule that codes for the peptides operatively linked to appropriate expression control sequences. Methods of affecting this operative linking, either before or after the DNA molecule is inserted into the vector, are well known. Expression control sequences include promoters, activators, enhancers, operators, ribosomal nuclease domains, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be suitable for use in the methods disclosed herein. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides since it is the most cost-effective method of making small peptides. Compounds that contain derivatized peptides or which contain non-peptide groups may be synthesized by well-known organic chemistry techniques.

Other methods are of molecule expression/synthesis are generally known in the art to one of ordinary skill.

### Expression of Polypeptides

The nucleic acid molecules described above can be contained within a vector that is capable of directing their expression in, for example, a cell that has been transduced with the vector. Accordingly, in addition to polypeptide mutants, expression vectors containing a nucleic acid molecule encoding a mutant and cells transfected with these vectors are among the certain embodiments.

Vectors suitable for use include T7-based vectors for use in bacteria (see, for example, Rosenberg et al., Gene 56: 125, 1987), the pMSXND expression vector for use in mammalian cells (Lee and Nathans, J. Biol. Chem. 263:3521, 1988), and baculovirus-derived vectors (for example the expression vector pBacPAKS from Clontech, Palo Alto, Calif.) for use in insect cells. The nucleic acid inserts, which encode the polypeptide of interest in such vectors, can be operably linked to a promoter, which is selected based on, for example, the cell type in which expression is sought. For example, a T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type- specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neoⁿ) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that are suitable for use include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a polypeptide mutant are also suitable for use. A cell is a transfected cell, i.e., a cell into which a nucleic acid molecule, for example a nucleic acid molecule encoding a mutant polypeptide, has been introduced by means of recombinant DNA techniques. The progeny of such a cell are also considered suitable for use in the methods disclosed herein.

The precise components of the expression system are not critical. For example, a polypeptide mutant can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., as therapeutic agents, as described herein.

### Pharmaceutical Compositions and Modes of Administration

In certain embodiments, a CD155/TIGIT antagonist (e.g., anti-CD155 antibody or anti-TIGIT antibody) and a TGF-β1 antagonist (e.g., a soluble form of a TGF-β receptor protein), are administered together (simultaneously or sequentially). In certain embodiments, the CD155/TIGIT antagonist (e.g., anti-CD155 antibody or anti-TIGIT antibody) is administered alone. In certain embodiments, the TGF-β1 antagonist (e.g., a soluble form of a TGF-β receptor protein) is administered alone.

In certain embodiments, the invention provides for a pharmaceutical composition comprising a CD155/TIGIT antagonist (e.g., anti-CD155 antibody or anti-TIGIT antibody) with a pharmaceutically acceptable diluent, carrier, solubilizer, emulsifier, preservative and/or adjuvant, and a pharmaceutical composition comprising a TGF-β1 antagonist (e.g., a soluble form of a TGF-β receptor protein) with a pharmaceutically acceptable diluent, carrier, solubilizer, emulsifier, preservative and/or adjuvant.

In certain embodiments, the invention provides for pharmaceutical compositions comprising a CD155/TIGIT antagonist (e.g., anti-CD155 antibody or anti-TIGIT antibody) and a TGF-β1 antagonist (e.g., a soluble form of a TGF-β receptor protein), with a pharmaceutically acceptable diluent, carrier, solubilizer, emulsifier, preservative and/or adjuvant. In certain embodiments, each of the agents, e.g., CD155/TIGIT antagonist (e.g., anti-CD155 antibody or anti-TIGIT antibody) and TGF-β1 antagonist (e.g., a soluble form of a TGF-β receptor protein), can be formulated as separate compositions. In certain embodiments, acceptable formulation materials preferably are nontoxic to recipients at the dosages and concentrations employed. In certain embodiments, the formulation material(s) are for s.c. and/or I.V. administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen- sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In certain embodiments, the formulation comprises PBS; 20 mM NaOAC, pH 5.2, 50 mM NaCl; and/or 10 mM NAOAC, pH 5.2, 9% Sucrose. In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the CD155/TIGIT antagonist (e.g., anti-CD155 antibody or anti-TIGIT antibody) and/or the TGF-β1 antagonist (e.g., a soluble form of a TGF-β receptor protein).

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising a CD155/TIGIT antagonist (e.g., anti-CD155 antibody or anti-TIGIT antibody) and/or a TGF-β1 antagonist (e.g., a soluble form of a TGF-β receptor protein), can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising a CD155/TIGIT antagonist (e.g., anti-CD155 antibody or anti-TIGIT antibody) and/or a TGF-β1 antagonist (e.g., a soluble form of a TGF-β receptor protein), can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising CD155/TIGIT antagonist (e.g., anti-CD155 antibody or anti-TIGIT antibody) and/or TGF-β1 antagonist (e.g., a soluble form of a TGF-β receptor protein), in a pharmaceutically acceptable vehicle. In certain embodiments, a vehicle for parenteral injection is sterile distilled water in which CD155/TIGIT antagonist (e.g., anti-CD155 antibody or anti-TIGIT antibody) and/or TGF-β1 antagonist (e.g., a soluble form of a TGF-β receptor protein), are formulated as a sterile, isotonic solution, and properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, CD155/TIGIT antagonist (e.g., anti-CD155 antibody or anti-TIGIT antibody) and/or TGF-β1 antagonist (e.g., a soluble form of a TGF-β receptor protein), can be formulated as a dry powder for inhalation. In certain embodiments, an inhalation solution comprising CD155/TIGIT antagonist (e.g., anti-CD155 antibody or anti-TIGIT antibody) and/or TGF-β1 antagonist (e.g., a soluble form of a TGF-β receptor protein), can be formulated with a propellant for aerosol delivery. In certain embodiments, solutions can be nebulized. Pulmonary administration is further described in PCT application No. PCT/US94/001875, which describes pulmonary delivery of chemically modified proteins.

In certain embodiments, it is contemplated that formulations can be administered orally. In certain embodiments, CD155/TIGIT antagonist (e.g., anti-CD155 antibody or anti-TIGIT antibody) and/or TGF-β1 antagonist (e.g., a soluble form of a TGF-β receptor protein), that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of CD155/TIGIT antagonist (e.g., anti-CD155 antibody or anti-TIGIT antibody) and/or TGF-β1 antagonist (e.g., a soluble form of a TGF-β receptor protein). In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of CD155/TIGIT antagonist (e.g., anti-CD155 antibody or anti-TIGIT antibody) and/or TGF-β1 antagonist (e.g., a soluble form of a TGF-β receptor protein), in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving CD155/TIGIT antagonist (e.g., anti-CD155 antibody or anti-TIGIT antibody) and/or TGF-β1 antagonist (e.g., a soluble form of a TGF-β receptor protein), in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15: 167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising CD155/TIGIT antagonist (e.g., anti-CD155 antibody or anti-TIGIT antibody) and/or TGF-β1 antagonist (e.g., a soluble form of a TGF-β receptor protein), to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which CD155/TIGIT antagonist (e.g., anti-CD155 antibody or anti-TIGIT antibody) and/or TGF-β1 antagonist (e.g., a soluble form of a TGF-β receptor protein), are being used, the route of administration, and the size (body weight, body surface or organ size) and/or condition (the age and general health) of the patient. In certain embodiments, the clinician can titer the dosage and modify the route of administration to obtain the optimal therapeutic effect.

In certain embodiments, the frequency of dosing will take into account the pharmacokinetic parameters of CD155/TIGIT antagonist (e.g., anti-CD155 antibody or anti-TIGIT antibody) and/or TGF-β1 antagonist (e.g., a soluble form of a TGF-β receptor protein), in the formulation used. In certain embodiments, a clinician will administer the composition until a dosage is reached that achieves the desired effect. In certain embodiments, the composition can therefore be administered as a single dose or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of the combination therapy may be administered by different routes.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration. In certain embodiments, it can be desirable to use a pharmaceutical composition comprising CD155/TIGIT antagonist (e.g., anti-CD155 antibody or anti-TIGIT antibody) and/or TGF-β1 antagonist (e.g., a soluble form of a TGF-β receptor protein), in an ex vivo manner. In such instances, cells, tissues and/or organs that have been removed from the patient are exposed to a pharmaceutical composition comprising CD155/TIGIT antagonist (e.g., anti-CD155 antibody or anti-TIGIT antibody) and/or TGF-β1 antagonist (e.g., a soluble form of a TGF-β receptor protein), after which the cells, tissues and/or organs are subsequently implanted back into the patient.

In certain embodiments, CD155/TIGIT antagonist (e.g., anti-CD155 antibody or anti-TIGIT antibody) and/or TGF-β1 antagonist (e.g., a soluble form of a TGF-β receptor protein), can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### Kits

A kit can include a CD155/TIGIT antagonist (e.g., anti-CD155 antibody or anti-TIGIT antibody) and a TGF-β1 antagonist (e.g., a soluble form of a TGF-β receptor protein), as disclosed herein, and instructions for use. The kits may comprise, in a suitable container, a CD155/TIGIT antagonist (e.g., anti-CD155 antibody or anti-TIGIT antibody), a TGF-β1 antagonist (e.g., a soluble form of a TGF-β receptor protein), one or more controls, and various buffers, reagents, enzymes and other standard ingredients well known in the art. Certain embodiments include a kit with a CD155/TIGIT antagonist (e.g., anti-CD155 antibody or anti-TIGIT antibody) and a TGF-β1 antagonist (e.g., a soluble form of a TGF-β receptor protein) in the same vial. In certain embodiments, a kit includes a CD155/TIGIT antagonist (e.g., anti-CD155 antibody or anti-TIGIT antibody) and a TGF-β1 antagonist (e.g., a soluble form of a TGF-β receptor protein) in separate vials.

The container can include at least one vial, well, test tube, flask, bottle, syringe, or other container means, into which a CD155/TIGIT antagonist (e.g., anti-CD155 antibody or anti-TIGIT antibody) and a TGF-β1 antagonist (e.g., a soluble form of a TGF-β receptor protein), may be placed, and in some instances, suitably aliquoted. Where an additional component is provided, the kit can contain additional containers into which this component may be placed. The kits can also include a means for containing a CD155/TIGIT antagonist (e.g., anti-CD155 antibody or anti-TIGIT antibody) and a TGF-β1 antagonist (e.g., a soluble form of a TGF-β receptor protein), and any other reagent containers in close confinement for commercial sale. Such containers may include injection or blow-molded plastic containers into which the desired vials are retained. Containers and/or kits can include labeling with instructions for use and/or warnings.

### Methods of Use

The CD155/TIGIT antagonist (e.g., anti-CD155 antibody or anti-TIGIT antibody) and TGF-β1 antagonist (e.g., a soluble form of a TGF-β receptor protein), and/or nucleic acids expressing them, described herein, are useful for treating a disorder associated with abnormal apoptosis or a differentiative process (e.g., cellular proliferative disorders (e.g., hyperproliferaetive disorders) or cellular differentiative disorders, such as cancer). Non-limiting examples of cancers that are amenable to treatment with the methods of the present invention are described below.

Examples of cellular proliferative and/or differentiative disorders include cancer (e.g., carcinoma, sarcoma, metastatic disorders or hematopoietic neoplastic disorders, e.g., leukemias). A metastatic tumor can arise from a multitude of primary tumor types, including but not limited to those of prostate, colon, lung, breast and liver. Accordingly, the compositions used herein, comprising, e.g., CD155/TIGIT antagonist (e.g., anti-CD155 antibody or anti-TIGIT antibody) and/or TGF-β1 antagonist (e.g., a soluble form of a TGF-β receptor protein), can be administered to a patient who has cancer.

In certain embodiments, a CD155/TIGIT antagonist (e.g., anti-CD155 antibody or anti-TIGIT antibody) is administered to a patient with cancer who has received or is receiving treatment with a TGF-β1 (e.g., a soluble form of a TGF-β receptor protein) antagonist, thereby treating the patient.

In certain embodiments, a TGF-β1 (e.g., a soluble form of a TGF-β receptor protein) antagonist is administered to a patient with cancer who has received or is receiving treatment with a CD155/TIGIT antagonist (e.g., anti-CD155 antibody or anti-TIGIT antibody), thereby treating the patient.

As described above, the compositions described herein (e.g., a CD155/TIGIT antagonist and a TGF-β antagonist) can be used to treat a variety of cancers such as but not limited to: leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer (including castration-resistant prostate cancer), squamous cell carcinoma, squamous cell carcinoma of the head and neck, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer(for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, cancer of the urinary system, and any other cancer type described herein (see, e.g., FIGS. 2A, 2B, and 2C) or otherwise known in the art.

In certain embodiments, the compositions described herein (e.g., a CD155/TIGIT antagonist and a TGF-β antagonist) can be used to treat colorectal cancer, breast cancer, stomach cancer, non-small cell lung cancer, cervical cancer, or pancreatic cancer.

In certain embodiments, the methods described herein further comprise a step of determining the level of CD155 expression in the tumor or cancer. In certain embodiments, the compositions described herein (e.g., a CD155/TIGIT antagonist and a TGF-β antagonist) can be used to treat cancer wherein the cancer cells express CD155. In certain embodiments, the compositions described herein (e.g., a CD155/TIGIT antagonist and a TGF-β antagonist) can be used to treat cancer wherein the cancer cells express elevated levels of CD155 relative to normal cells (i.e., not cancer cells) of the same histological type. In some embodiments, the elevated expression level of CD155 is in comparison to the levels in a reference sample. In some embodiments, the elevated expression level of CD155 is in comparison to a pre-determined level of CD155. In some embodiments, the elevated expression level of CD155 is in comparison to PD-L1. In some embodiments, the term “elevated” is equivalent to the terms “overexpress,” “increased” and similar terms. In some embodiments, monoclonal and polyclonal antibodies that bind CD155 can be used to assay the expression level of CD155. For example, in some embodiments, an anti-CD155 antibody is a monoclonal antibody, such as D171. Methods for assaying for the presence of CD155 are described in U.S. Pat. No. 6,518,033, the entire contents of which are incorporated herein by reference. (U.S. Pat. No. 6,518,033) Antibodies that bind CD155, and methods of making and using the same are described in US2014/056890, the entire contents of which is incorporated herein by reference.

In certain embodiments, the methods described herein further comprise a step of determining the level of TIGIT expression by immune cells (e.g., tumor infiltrating lymphocytes) or cells of the tumor or cancer. In certain embodiments, the compositions described herein (e.g., a CD155/TIGIT antagonist and a TGF-β antagonist) can be used to treat cancer wherein the cancer cells express TIGIT. In some embodiments, TIGIT expression is measured on activated T cells (regulatory T cells (T_{reg}), memory T cells, and follicular B cell helper T cells (T_{fh})) and NK cells. In some embodiments, TIGIT expression is measured on tumor infiltrating lymphocytes (TILs). In certain embodiments, the compositions described herein (e.g., a CD155/TIGIT antagonist and a TGF-β antagonist) can be used to treat cancer wherein the cancer cells express elevated levels of TIGIT relative to normal cells (i.e., not cancer cells) of the same histological type. In some embodiments, the elevated expression level of TIGIT is in comparison to the levels in a reference sample. In some embodiments, the elevated expression level of TIGIT is in comparison to a pre-determined level of TIGIT. In some embodiments, the elevated expression level of TIGIT is in comparison to PD-1.

In certain embodiments, the methods described herein further comprise a step of determining the level of expression of a TGF-β signaling component (e.g., TGF-β1) by immune cells (e.g., tumor infiltrating lymphocytes) or cells of the tumor or cancer. In certain embodiments, the compositions described herein (e.g., a CD155/TIGIT antagonist and a TGF-β antagonist) can be used to treat cancer wherein the cancer cells express a TGF-β signaling component (e.g., TGF-β1). In certain embodiments, the compositions described herein (e.g., a CD155/TIGIT antagonist and a TGF-β antagonist) can be used to treat cancer wherein the cancer cells express elevated levels of a TGF-β signaling component (e.g., TGF-β1) relative to normal cells (i.e., not cancer cells) of the same histological type. In some embodiments, the elevated expression level of a TGF-β signaling component (e.g., TGF-β1) is in comparison to the levels in a reference sample. In some embodiments, the elevated expression level of a TGF-β signaling component (e.g., TGF-β1) is in comparison to a pre-determined level of the TGF-β signaling component (e.g., TGF-β1).

In certain embodiments, the disclosure provides methods for treating cancer comprising: i) obtaining the results of an assay, wherein a test tissue sample obtained from a subject with a cancer tissue is assessed for expression of CD155, TIGIT or a TGF-β signaling component (e.g., TGF-β1); ii) administering to a subject diagnosed with cancer a therapeutically effective amount of a CD155/TIGIT antagonist and a therapeutically effective amount of a TGF-β1 antagonist, wherein expression of CD155, TIGIT or a TGF-β signaling component (e.g., TGF-β1) in the test tissue exceeds a predetermined level. In certain embodiments, the test tissue sample comprises tumor cells and tumor-infiltrating inflammatory cells. In certain embodiments, the assay assesses a proportion of cells in the test tissue sample for expression of CD155, TIGIT or a TGF-β signaling component (e.g., TGFβRII) on a cell surface.

Methods for determining the level of expression of CD155, TIGIT or a TGF-β signaling component (e.g., TGF-β1) in a cell, tumor, or cancer are known by those of skill in the art. For nucleic acid expression, these methods include, but are not limited to, PCR-based assays, microarray analyses, and nucleotide sequencing (e.g., NextGen sequencing). Additional methods include determining gene expression in various tissues by Southern blotting, Northern blotting to quantitate the transcription of mRNA (Thomas, PNAS 77: 5201-5205, 1980), dot blotting (DNA analysis) or in situ hybridization, using an appropriately labeled probe, based on the sequences of CD155, TIGIT or TGF-β signaling component (e.g., TGF-β1). Alternatively, antibodies may be employed that can recognize specific duplexes, including DNA duplexes, RNA duplexes, and DNA-RNA hybrid duplexes or DNA-protein duplexes.

For protein expression, these methods include, but are not limited to, Western blot analysis, protein arrays, immunohistochemistry (IHC) assays, immunoprecipitation, immunoblot, enzyme-linked immunosorbant assays (ELISA) and FACS. For example, immunohistochemical staining of tissue sections and assay of cell culture or body fluids are used to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence of a polypeptide or against a synthetic peptide based on the DNA sequences encoding the polypeptide or against an exogenous sequence fused to a DNA encoding a polypeptide and encoding a specific antibody epitope. In some embodiments, protein expression is determined by the integrated geometic mean fluorescence intensity (iGMFI), which is a relative quantitative measure of the intensity of the marker expression on a particular cell type, calculated by multiplying the frequency of the cell population by the geometric fluorescence intensity of the marker.

Methods for determining whether a tumor or cancer has an elevated level of expression of CD155, TIGIT or a TGF-β signaling component (e.g., TGF-β1) can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a processed cell lysate. In some embodiments, the sample is processed to DNA or RNA. In some embodiments, the sample is peripheral blood mononuclear cells (PBMCs). In some embodiments, the sample is dissociated tumor cells. In some embodiments, the sample is ascites. In some embodiments, tumor cells, as determined by CD45-EpCAM+ staining, are measured for expression. In some embodiments, immune cells, as determined by CD45+EpCAM− staining, are measured for expression. In some embodiments, stroma cells, as determined by CD45-EpCAM− staining, are measured for expression.

In some embodiments, CD155 expression is higher than PD-L1 expression on tumor cells (CD45-EpCAM+), immune cells (CD45+EpCAM−) and stroma cells (CD45-EpCAM−). In some embodiments, CD155 expression is higher than PD-L1 expression on tumor cells (CD45-EpCAM+), immune cells (CD45+EpCAM−) and stroma cells (CD45-EpCAM−) present in ovarian cancer ascites. In some embodiments, CD155 expression is higher than PD-L1 expression on tumor cells (CD45-EpCAM+) in patients having colorectal cancer. In some embodiments, CD155 expression is higher than PD-L1 expression on tumor cells (CD45-EpCAM+) in patients having non-small cell lung carcinoma. In some embodiments, CD155 expression is higher than PD-L1 expression on tumor cells (CD45-EpCAM+) in patients having ovarian cancer.

In some embodiments, TIGIT expression is higher than PD-1 expression in CD4+ PBMCs, CD4+ tumor infiltrating lymphocytes (TILs), CD8^{high} PBMCs, CD8^{high} TILs, CD81^{low} PBMCs and CD8^{low} TILs from a patient with cancer. In some embodiments, TIGIT expression is higher than PD-1 expression in CD4+ PBMCs from a patient with cancer. In some embodiments, TIGIT expression is higher than PD-1 expression in CD4+ TILs from a patient with cancer. In some embodiments, TIGIT expression is higher than PD-1 expression in CD8^{high} PBMCs from a patient with cancer. In some embodiments, TIGIT expression is higher than PD-1 expression in CD8^{high} TILs from a patient with cancer. In some embodiments, TIGIT expression is higher than PD-1 expression in CD8^{low} PBMCs from a patient with cancer. In some embodiments, TIGIT expression is higher than PD-1 expression in CD8^{lows} TILs from a patient with cancer.

It will be appreciated by those skilled in the art that amounts for each of the CD155/TIGIT antagonist (e.g., anti-CD155 antibody or anti-TIGIT antibody) and/or TGF-β1 antagonist (e.g., a soluble form of a TGF-β receptor protein), that are sufficient to reduce tumor growth and size, or a therapeutically effective amount, will vary not only on the particular compounds or compositions selected, but also with the route of administration, the nature of the condition being treated, and the age and condition of the patient, and will ultimately be at the discretion of the patient's physician or pharmacist. The length of time during which the compounds used in the instant method will be given varies on an individual basis.

In certain embodiments, the CD155/TIGIT antagonist (e.g., anti-CD155 antibody or anti-TIGIT antibody) and/or TGF-β1 antagonist (e.g., a soluble form of a TGF-β receptor protein) disclosed herein are used to treat cancer. In certain embodiments, the CD155/TIGIT antagonist (e.g., anti-CD155 antibody or anti-TIGIT antibody) and/or TGF-β1 antagonist (e.g., a soluble form of a TGF-β receptor protein) disclosed herein induce a durable clinical response in a patient.

In certain embodiments, the CD155/TIGIT antagonist (e.g., anti-CD155 antibody or anti-TIGIT antibody) and/or TGF-β1 antagonist (e.g., a soluble form of a TGF-β receptor protein) disclosed herein are used to treat melanoma, leukemia, lung cancer, breast cancer, prostate cancer, ovarian cancer, colon cancer, and brain cancer.

In certain embodiments, the CD155/TIGIT antagonist (e.g., anti-CD155 antibody or anti-TIGIT antibody) and/or TGF-β1 antagonist (e.g., a soluble form of a TGF-β receptor protein) disclosed herein inhibit the growth and/or proliferation of tumor cells.

In certain embodiments, the CD155/TIGIT antagonist (e.g., anti-CD155 antibody or anti-TIGIT antibody) and/or TGF-β1 antagonist (e.g., a soluble form of a TGF-β receptor protein) disclosed herein reduce tumor size.

In certain embodiments, the CD155/TIGIT antagonist (e.g., anti-CD155 antibody or anti-TIGIT antibody) and/or TGF-β1 antagonist (e.g., a soluble form of a TGF-β receptor protein) disclosed herein inhibit metastases of a primary tumor.

The development of cancer is often a result of the immune system's failure to adequately recognize and respond to the presence of cancer cells, cancer antigens, and/or tumors. Therefore, inducing or enhancing a cancer-specific immune response in a patient is beneficial and in some instances will treat the cancer. In certain embodiments, a cancer-specific immune response involves an increase in T cell proliferation and function (i.e., a T cell response). In certain embodiments, a cancer-specific immune response involves an increase in tissue infiltrating lymphocytes (TILs). In certain embodiments, a cancer-specific immune response involves an increase in cytotoxic T cells. In certain embodiments, a cancer-specific immune response involves the production of antibodies against cancer cells, cancer antigens, and/or tumors.

In certain embodiments, the CD155/TIGIT antagonist (e.g., anti-CD155 antibody or anti-TIGIT antibody) and TGF-β1 antagonist (e.g., a soluble form of a TGF-β receptor protein) disclosed herein promote a cancer-specific immune response in a patient. In certain embodiments, the disclosure provides methods for enhancing a cancer-specific immune response in a patient who has received or is receiving treatment with a TGF-β1 antagonist, comprising administering an effective amount of a CD155/TIGIT antagonist. In some embodiments, a patient has received TGF-β1 antagonist treatment any length of time (e.g. 1 year, 6 months, 1 day) before receiving the CD155/TIGIT antagonist. In some embodiments, the TGF-β1 antagonist is no longer biologically active within the patient, based on the pre-determined pharmacokinetics of the TGF-β1 antagonist, when the CD155/TIGIT antagonist is administered. In some embodiments, a patient has received TGF-β1 antagonist treatment the same day as the CD155/TIGIT antagonist but before the CD155/TIGIT antagonist is administered. In some embodiments, a patient receives TGF-β1 antagonist treatment prior to receiving a CD155/TIGIT antagonist. In some embodiments, a patient receiving TGF-β1 antagonist treatment is administered a CD155/TIGIT antagonist simultaneously, concurrently, or sequentially.

In certain embodiments, the disclosure provides methods for enhancing a cancer-specific immune cell response in a patient who has received or is receiving treatment with a CD155/TIGIT antagonist, comprising administering an effective amount of a TGF-β1 antagonist. In some embodiments, a patient has received CD155/TIGIT antagonist treatment any length of time (e.g. 1 year, 6 months, 1 day) before receiving the TGF-β1 antagonist. In some embodiments, the CD155/TIGIT antagonist is no longer biologically active within the patient, based on the pre-determined pharmacokinetics of the CD155/TIGIT antagonist, when the TGF-β1 antagonist is administered. In some embodiments, a patient has received CD155/TIGIT antagonist treatment the same day as the TGF-β1 antagonist but before the TGF-β1 antagonist is administered. In some embodiments, a patient receives CD155/TIGIT antagonist treatment prior to receiving a TGF-β1 antagonist. In some embodiments, a patient receiving CD155/TIGIT antagonist treatment is administered a TGF-β1 antagonist simultaneously, concurrently, or sequentially.

In certain embodiments, the disclosure provides methods for enhancing a cancer-specific immune cell response in a patient comprising administering an effective amount of a CD155/TIGIT antagonist and a TGF-β1 antagonist. In some embodiments, the patient is administered an effective about of a CD155/TIGIT antagonist and a TGF-β1 antagonist simultaneously, concurrently, or sequentially.

In certain embodiments, the cancer-specific immune response in a patient is greater than the cancer-specific immune response following administration of either the CD155/TIGIT antagonist or TGF-β1 antagonist alone. In certain embodiments, the cancer-specific immune response comprises the production of IFNγ by one or both of CD4+ T cells and CD8+ T cells. In certain embodiments, the cancer-specific immune response comprises the production of IL-2 by one or both of CD4+ T cells and CD8+ T cells.

In certain embodiments, the cancer-specific immune response is a T cell response. In certain embodiments, the T cell response in a patient is greater than the T cell response following administration of either the CD155/TIGIT antagonist or TGF-β1 antagonist alone. In certain embodiments, the T cell response comprises the production of IFNγ by one or both of CD4+ T cells and CD8+ T cells. In certain embodiments, the T cell response comprises the production of IL-2 by one or both of CD4+ T cells and CD8+ T cells.

Effective treatment of cancer (and thus the reduction thereof) can be detected by any variety of suitable methods. Methods for detecting cancers and effective cancer treatment include clinical examination (symptoms can include swelling, palpable lumps, enlarged lymph nodes, bleeding, visible skin lesions, and weight loss); imaging (X-ray techniques, mammography, colonoscopy, computed tomography (CT and/or CAT) scanning, magnetic resonance imaging (MRI), etc.); immunodiagnostic assays (e.g., detection of CEA, AFP, CA125, etc.); antibody-mediated radioimaging; and analyzing cellular/tissue immunohistochemistry. Other examples of suitable techniques for assessing a cancerous state and effective cancer treatment include PCR and RT-PCR (e.g., of cancer cell associated genes or “markers”), biopsy, electron microscopy, positron emission tomography (PET), computed tomography, magnetic resonance imaging (MRI), karyotyping and other chromosomal analysis, immunoassay/immunocytochemical detection techniques (e.g., differential antibody recognition), histological and/or histopathologic assays (e.g., of cell membrane changes), cell kinetic studies and cell cycle analysis, ultrasound or other sonographic detection techniques, radiological detection techniques, flow cytometry, endoscopic visualization techniques, and physical examination techniques.

In other aspects, the disclosure provides methods for treating chronic infectious diseases by administering a combination of CD155/TIGIT antagonist and TGF-β1 antagonist, as described herein. Long-term persistence of an infectious agent (e.g., bacteria, virus, and fungus) results in a chronic infection. As disclosed herein, a combination of CD155/TIGIT antagonist and TGF-β1 antagonist leads to T cell activation (i.e., production of IFNγ and IL-2). At least because Th1 cytokine production by T cells is associated with productive immune responses, administration of a CD155/TIGIT pathway antagonist in combination with a TGF-β1 antagonist is useful for treating chronic infectious diseases.

In some embodiments, a CD155/TIGIT antagonist and a TGF-β1 antagonist are useful in treating poliovirus infection. Polioviruses belong to the Picornaviridae family, which includes viruses composed of a single-stranded, positive-sense RNA genome and a protein capsid. Infection with poliovirus results in poliomyelitis, a neurological disease. Since the poliovirus receptor (PVR) is CD155, in some embodiments antagonizing the CD155/TIGIT pathway prevents continued infection. In some embodiments, a CD155/TIGIT antagonist and a TGF-β1 antagonist inhibit chronic infection of the poliovirus. In some embodiments, a CD155/TIGIT antagonist and a TGF-β1 antagonist inhibit chronic infection of the poliovirus and enhance an immune response. In some embodiments, the CD155/TIGIT antagonist and the TGF-β1 antagonist disclosed herein treat a poliovirus infection.

It will be appreciated by those skilled in the art that reference herein to treatment extends to prophylaxis as well as the treatment of the noted cancers and symptoms.

### EXAMPLES

### Example 1

### Analysis of Immune Modulator Expression in Various Cancers

To determine whether expression levels of known immune modulators are altered in cancer, a bio-informatics mining of the data available in the public domain was conducted using The Cancer Genome Atlas (TCGA). Using the cBioportal database, which harbors data on 21441 tumor samples from 91 cancer studies, the frequency of altered genes (amplification, mutation and deletion) in the TCGA studies was assessed. Specifically, the frequency in the alterations of individual genes TIGIT, CD155 and TGF-β was analyzed. The results in FIGS. 2A-2C showed fewer deletions of these genes compared to mutations or amplifications in cancer patients, therefore indicating a viable pathway to target in a broad spectrum of cancer types, including those described in FIGS. 2A-2C.

Expression levels of PD-L1, CD155, TIGIT, PD-1 and TGF-β messenger RNA (mRNA) were also assessed across different human cancers (i.e., colorectal cancer (CRC), breast cancer, cervical cancer, stomach cancer, non-small cell lung carcinoma (NSCLC), and pancreatic cancer) relative to normal healthy individuals. mRNA expression z-scores for each cancer were downloaded from the TCGA database and graphed using Graphpad Prism (see FIG. 3). The z-scores were generated by TCGA using RNA Seq Ver2 RSEM (RNA-Seq by Expectation Maximization; Illumina). Expression of CD155 was also measured in various human cancer cell lines. Cells were thawed, washed and rested overnight and stained for flow cytometry with antibody specific for CD155. The stained cells were acquired on a BD LSRFortessa and analyzed using Flowjo software. FIG. 4 shows that CD155 was expressed on several cancer cell lines, including those of the lymphoid population (i.e., Burkitt's Lymphoma). Normally, cells in the lymphoid population do not express adhesion type molecules, such as CD155.

Taken together, these results indicate CD155 and TIGIT are highly expressed on multiple cancer types.

### Example 2

### Assessment of Immune Modulators in Samples from Cancer Patients

Expression of CD155, PD-L1, TIGIT and PD-1 in samples from healthy donors or patients having colorectal cancer (CRC), ovarian cancer or lung cancer was analyzed. The lung cancer samples are from a mixed population, including non-small cell lung carcinoma (NSCLC). The characteristics of the samples analyzed are shown in the Table below:

| Samples (n) | Diagnosis (n) | Cell types (n) |
|---|---|---|
| Cancer Patients (n = 32) | Colorectal cancer (n = 12) | PBMCs (n = 6) |
| | | DTC (n = 6) |
| | Lung cancer (n = 12) | PBMCs (n = 6) |
| | | DTC (n = 6) |
| | Ovarian Cancer (n = 18) | Ascites (n = 6) |
| | | PBMCs (n = 6) |
| | | DTC (n = 6) |
| Normal Donors (n = 11) | Healthy (n = 11) | PBMCs (n = 11) |

DTC = dissociated tumor cells,
PBMCs = peripheral blood mononuclear cells

Dissociated tumor cells (DTCs), peripheral blood mononuclear cells (PBMCs) and/or ascites cells from ovarian cancer, CRC and NSCLC were obtained from Conversant Bio, along with PBMCs from normal healthy donors. Cells were thawed, washed and rested overnight to allow the recovery of surface marker expression. Cells were then stained with antibody specific markers for immune cells (i.e., CD3, CD28 CD4, CD8 and CD45), tumor cells (CD45-EpCAM+), PD-L1, CD155, PD-1 and TIGIT, which were measured by flow cytometry using the BD LSRFortessa cytometer and analyzed using Flowjo software. Antibodies for CD3, CD28, CD45, CD155, EpCAM, and PD-L1 were obtained from BioLegend, whereas anti-PD-1 was obtained from Bioscience and anti-TIGIT was obtained from EBioscience. For PD-L1 and CD155, the integrated geometric mean fluorescence intensity (iGMFI), a relative quantitative measure of the intensity of the marker expression on a particular cell type, was calculated by multiplying the frequency of the cell population by the geometric fluorescence intensity of the marker. For PD-1 and TIGIT, the frequencies of positive events were compared across different indications.

FIG. 5 shows that CD155 expression was higher than PD-L1 expression on tumor cells (CD45-EpCAM+), immune cells (CD45+EpCAM−) and stroma cells (CD45-EpCAM−) present in ovarian cancer ascites. FIG. 6 shows that CD155 expression was also higher than PD-L1 expression on tumor cells (CD45-EpCAM+) isolated from DTCs from patients having CRC, lung or ovarian cancers.

FIG. 7 shows TIGIT expression compared to PD-1 expression in cells isolated from DTC and PBMC samples. Specifically, CD4+ PBMCs, CD4+ tumor infiltrating lymphocytes (TILs), CD8^{high} PBMCs, CD8^{high} TILs, CD8^{low} PBMCs and CD8^{low} TILs. TILs were isolated from the DTC samples of the indicated cancers. TIGIT was found to be more highly expressed than PD-1 on CD8+ and CD4+ TILs in CRC, lung and ovarian cancers.

### Example 3

### Effect of Blockade of Immune Modulators

It was next assessed whether antibody blockade of CD155 or TIGIT had an effect on cytokine production by T cells. MDA-MB321 cells (ATCC® HTB-26; human breast adenocarcinoma cell line), which express CD155, were co-cultured with activated human primary T cells isolated from PBMCs of healthy donors. Specifically, 5×10⁴ MDA-MB231 cells were plated in a 96-well plate and allowed to rest for 24 hours, whereas T cells were stimulated with soluble anti-human CD3 (BioLegend; clone OKT3) and anti-human CD28 (BioLegend; clone CD28.2) at a final concentration of 10 μg/ml and 1 μg/ml respectively, and then added to the MDA-MB231 cells at 3:1 ratio (T cells:tumor cells). The cells were then incubated for 4 hours and a brefeldin A/monensin (Sigma) combination was added for the last 12 hours. Anti-CD155 (mouse anti-human; clone SKII.4, BioLegend), anti-TIGIT (clone MBSA43, eBioscience), and mouse IgG1 (eBioscience) were added individually to the cultures when the cells were combined. All the blocking or isotype control antibodies were used at a final concentration of 5 μg/ml. The following flow cytometry antibodies were used: AF488-CD3, BV421-CD4, APC-Cy7-CD8, PE-Cy7-IFN-γ (clone 4S.B3; eBioscience) and PerCp-eFluor 710 (clone MQ1-17H12; eBioscience).

After staining, the cells were analyzed for cytokine production on a BD LSRII and analyzed using Flowjo software. The results, shown in FIGS. 8 (CD155 blockade) and 9 (TIGIT blockade), showed that antibody blockade of CD155 and TIGIT promoted T cell activation of both CD4+ and CD8+ T cells, as measured IFN-γ and IL-2 production.

Using the same system, it was next assessed whether blockade of the CD155/TIGIT pathway in combination with TGF-β neutralization further enhanced T cell activation. To neutralize TGF-β, a TGF-β receptor II (TGFRII) fused to an Fc (TGFRII-Fc; 341-BR R&D Systems) was utilized at a concentration of 2.5 μg/ml. The cells were incubated for 72 hours and the brefeldin A/monensin combination was added for the last 12 hours. The anti-CD155 or anti-TIGIT antibodies were added alone or in combination with TGFRII-Fc when the cells were mixed. The cells were stained with the same flow cytometry antibodies described above and analyzed for cytokine production on a BD LSRII and analyzed using Flowjo software.

The results of combining anti-CD155 with TGFRII-Fc are shown in FIG. 10. Combination of CD155 antibody blockade with TGF-β neutralization via a TGFBRII-Fc protein resulted in enhanced IFN-γ and IL-2 production by CD4+ and CD8+ T cells as compared to either CD155 or TGF-β neutralization alone. The results of combining anti-TIGIT with TGFRII-Fc are shown in FIG. 11. As with combination of anti-CD155 and TGFRII-Fc, combination of TIGIT antibody blockade with TGF-β neutralization via a TGFBRII-Fc protein resulted in enhanced IFN-γ and IL-2 production by CD4+ and CD8+ T cells as compared to either TIGIT or TGF-β neutralization alone.

These results confirm simultaneously neutralizing CD155/TIGIT and TGF-β pathways enhances T cell activation as compared to individually neutralizing these pathways.

### Equivalents

Those skilled in the art will recognize or be able to ascertain, using no more than routine experimentation, many equivalents of the specific embodiments described herein. Such equivalents are intended to be encompassed by the following claims.

TABLE 1
Summary Table of Sequences

| SEQ | | |
|---|---|---|
| ID | | |
| NO | Description | Sequence |
| 1 | Human IgG1 | ASTKGPSVFPLAPSSKSTSGGTAALGCLVKDYFPEPVTVSWNSGA |
| | constant | LTSGVHTFPAVLQSSGLYSLSSVVTVPSSSLGTQTYICNVNHKPSN |
| | region | TKVDKKVEPKSCDKTHTCPPCPAPELLGGPSVFLFPPKPKDTLMIS |
| | (amino acid | RTPEVTCVVVDVSHEDPEVKFNWYVDGVEVHNAKTKPREEQYN |
| | sequence) | STYRVVSVLTVLHQDWLNGKEYKCKVSNKALPAPIEKTISKAKG |
| | | QPREPQVYTLPPSRDELTKNQVSLTCLVKGFYPSDIAVEWESNGQ |
| | | PENNYKTTPPVLDSDGSFFLYSKLTVDKSRWQQGNVFSCSVMHE |
| | | ALHNHYTQKSLSLSPGK |
| 2 | Human IgG1 | PKSCDKTHTCPPCPAPELLGGPSVFLFPPKPKDTLMISRTPEVTCV |
| | Fc domain | VVDVSHEDPEVKFNWYVDGVEVHNAKTKPREEQYNSTYRVVSV |
| | (amino acid | LTVLHQDWLNGKEYKCKVSNKALPAPIEKTISKAKGQPREPQVY |
| | sequence) | TLPPSRDELTKNQVSLTCLVKGFYPSDIAVEWESNGQPENNYKTT |
| | | PPVLDSDGSFFLYSKLTVDKSRWQQGNVFSCSVMHEALHNHYTQ |
| | | KSLSLSPGK |
| 3 | Human | MGRGLLRGLWPLHIVLWTRIASTIPPHVQKSDVEMEAQKDEIICP |
| | TGFβRII | SCNRTAHPLRHINNDMIVTDNNGAVKFPQLCKFCDVRFSTCDNQ |
| | Isoform A | KSCMSNCSITSICEKPQEVCVAVWRKNDENITLETVCHDPKLPYH |
| | Precursor | DFILEDAASPKCIMKEKKKPGETFFMCSCSSDECNDNIIFSEEYNTS |
| | Polypeptide | NPDLLLVIFQVTGISLLPPLGVAISVIIIFYCYRVNRQQKLSSTWET |
| | (NCBI | GKTRKLMEFSEHCAIILEDDRSDISSTCANNINHNTELLPIELDTLV |
| | RefSeq | GKGRFAEVYKAKLKQNTSEQFETVAVKIFPYEEYASWKTEKDIFS |
| | Accession | DINLKHENILQFLTAEERKTELGKQYWLITAFHAKGNLQEYLTRH |
| | No: | VISWEDLRKLGSSLARGIAHLHSDHTPCGRPKMPIVHRDLKSSNIL |
| | NP | VKNDLTCCLCDFGLSLRLDPTLSVDDLANSGQVGTARYMAPEVL |
| | 001020018) | ESRMNLENVESFKQTDVYSMALVLWEMTSRCNAVGEVKDYEPP |
| | | FGSKVREHPCVESMKDNVLRDRGRPEIPSFWLNHQGIQMVCETL |
| | | TECWDHDPEARLTAQCVAERFSELEHLDRLSGRSCSEEKIPEDGS |
| | | LNTTK |
| 4 | Human | MGRGLLRGLWPLHIVLWTRIASTIPPHVQKSVNNDMIVTDNNGA |
| | TGFβRII | VKFPQLCKFCDVRFSTCDNQKSCMSNCSITSICEKPQEVCVAVWR |
| | Isoform B | KNDENITLETVCHDPKLPYHDFILEDAASPKCIMKEKKKPGETFF |
| | Precursor | MCSCSSDECNDNIIFSEEYNTSNPDLLLVIFQVTGISLLPPLGVAISV |
| | Polypeptide | IIIFYCYRVNRQQKLSSTWETGKTRKLMEFSEHCAIILEDDRSDISS |
| | (NCB I | TCANNINHNTELLPIELDTLVGKGRFAEVYKAKLKQNTSEQFETV |
| | RefSeq | AVKIFPYEEYASWKTEKDIFSDINLKHENILQFLTAEERKTELGKQ |
| | Accession | YWLITAFHAKGNLQEYLTRHVISWEDLRKLGSSLARGIAHLHSD |
| | No: NP | HTPCGRPKMPIVHRDLKSSNILVKNDLTCCLCDFGLSLRLDPTLSV |
| | 003233 | DDLANSGQVGTARYMAPEVLESRMNLENVESFKQTDVYSMALV |
| | | LWEMTSRCNAVGEVKDYEPPFGSKVREHPCVESMKDNVLRDRG |
| | | RPEIPSFWLNHQGIQMVCETLTECWDHDPEARLTAQCVAERFSEL |
| | | EHLDRLSGRSCSEEKIPEDGSLNTTK |
| 5 | Extracellular | TIPPHVQKSVNNDMIVTDNNGAVKFPQLCKFCDVRFSTCDNQKS |
| | domain of | CMSNCSITSICEKPQEVCVAVWRKNDENITLETVCHDPKLPYHDF |
| | Human | ILEDAASPKCIMKEKKKPGETFFMCSCSSDECNDNIIFSEEYNTSNP |
| | TGFβRII | D |
| | (Thr23- | |
| | Asp159) | |
| | amino acid | |
| | sequence | |
| 6 | Extracellular | GGATCCACCATCCCCCCACACGTGCAGAAATCCGTGAACAAC |
| | domain of | GACATGATCGTGACCGACAACAACGGCGCTGTGAAGTTCECCC |
| | Type 2 TGF- | AGCTGTGCAAGTTCTGCGAEGTGCGGTTCTCTACCTGCGACAA |
| | β receptor | CCAGAAATCCTGCATGTCCAACTGCTCCATCACCTCCATETGC |
| | nucleic acid | GAGAAGCCCCAGGAAGTGTGCGTCGCCGTCTGGCGGAAGAAC |
| | sequence | GACGAGAACATCACCETGGAAACCGTGTGCCACGACCCCAAG |
| | | CTGCCCTACCACGACTTCATCCTGGAAGATGCCGCCTCCCCCA |
| | | AGTGCATCATGAAGGAAAAGAAGAAGCCCGGCGAGACTTTCT |
| | | TCATGTGCAGCTGCTCCTCCGACGAGTGCAACGACAACATCAT |
| | | CTTCTCCGAAGAGTACAACACCTCCAACCCEGACTGAAGCTT |

1. A method for treating cancer in a patient, the method comprising: administering to the patient an effective amount of: (i) a CD155/TIGIT antagonist; and (ii) a TGF-β1 antagonist, thereby treating the patient.

2. A method for treating cancer in a patient who has received or is receiving treatment with a TGF-β1 antagonist, the method comprising: administering to the patient an effective amount of a CD155/TIGIT antagonist, thereby treating the patient.

3. A method for treating cancer in a patient who has received or is receiving treatment with a CD155/TIGIT antagonist, the method comprising: administering to the patient an effective amount of a TGF-β1 antagonist, thereby treating the patient.

4. The method of any one of claims 1 to 3, wherein cancer cells of the cancer express CD155.

5. The method of claim 4, wherein the cancer cells overexpress CD155 relative to normal cells of the same histological type.

6. The method of any one of claims 1 to 5, further comprising determining whether cancer cells of the cancer express or overexpress CD155.

7. The method of any one of claims 1 to 6, wherein the CD155/TIGIT antagonist is an anti-CD155 antibody or an antigen-binding fragment thereof.

8. The method of any one of claims 1 to 6, wherein the CD155/TIGIT antagonist is an anti-TIGIT antibody or an antigen-binding fragment thereof.

9. The method of claim 7 or 8, wherein the antibody is a humanized antibody or a fully human antibody.

10. The method of any one of claims 1 to 9, wherein the TGF-β1 antagonist comprises a small molecule, nucleic acid or polypeptide.

11. The method of claim 10, wherein the TGF-β1 antagonist is a soluble form of a TGF-β receptor protein.

12. The method of claim 11, wherein the TGF-β receptor protein is an extracellular domain of the type 2 TGF-β receptor or a TGF-β1-binding fragment thereof.

13. The method of claim 12, wherein the TGF-β receptor protein comprises the amino acid sequence depicted in SEQ ID NO: 5.

14. The method of any one of claims 11 to 13, wherein the TGF-β1 antagonist comprises a fusion protein comprising an extracellular domain of the type 2 TGF-β receptor, or a TGF-β1-binding fragment thereof, and a moiety that enhances the serum half-life of the TGF-β1 antagonist.

15. The method of claim 14, wherein the moiety comprises polyethylene glycol, an Fc portion of an antibody, or an albumin protein.

16. The method of claim 15, wherein the TGF-β1 antagonist comprises a fusion protein comprising an extracellular domain of the type 2 TGF-β receptor or a TGF-β1-binding fragment thereof and a human IgG Fc domain.

17. The method of claim 16, wherein the human IgG Fc domain is a human IgG1 Fc domain, a human IgG2 Fc domain, a human IgG3 Fc domain, or a human IgG4 Fc domain.

18. The method of claim 16 or 17, wherein the TGF-β1 antagonist comprises (a) an extracellular domain of the type 2 TGF-β receptor comprising the amino acid sequence depicted in SEQ ID NO: 5, and (b) a human IgG1 Fc domain comprising the amino acid sequence depicted in SEQ ID NO: 2.

19. The method of any of the preceding claims, wherein treatment comprises a cancer-specific immune response in the patient.

20. The method of claim 19, wherein the cancer-specific immune response is a T cell response.

21. The method of claim 20, wherein the T cell response in the patient is greater than the T cell response following administration of either the CD155/TIGIT antagonist or the TGF-β1 antagonist alone.

22. The method of claim 20 or 21, wherein the T cell response comprises the production of IFNγ by one or both of CD4⁺ T cells and CD8⁺ T cells.

23. The method of any one of claims 20 to 22, wherein the T cell response comprises the production of IL-2 by one or both of CD4⁺ T cells and CD8⁺ T cells.

24. The method of any one of claims 20 to 23, wherein the T cell response comprises proliferation of one or both of CD4⁺ T cells and CD8⁺ T cells.

25. The method of any of the preceding claims, wherein treatment comprises delaying cancer progression in the patient.

26. The method of any of the preceding claims, wherein treatment comprises enhancing a tumor-specific immune response in the patient.

27. A method for treating cancer in a patient, the method comprising: administering to the patient an effective amount of:
(i) a CD155/TIGIT antagonist, wherein the CD155/TIGIT antagonist is an anti-CD155 antibody or antigen-binding fragment thereof, or an anti-TIGIT antibody or antigen-binding fragment thereof; and
(ii) a TGF-β1 antagonist, wherein the TGF-β1 antagonist comprises a fusion protein comprising an extracellular domain of the type 2 TGF-β receptor or a TGF-β1-binding fragment thereof and a human IgG1 Fc domain,
thereby treating the patient.

28. A method for enhancing a cancer-specific immune response in a cancer patient who has received or is receiving treatment with a TGF-β1 antagonist, the method comprising:
administering to the patient an effective amount of a CD155/TIGIT antagonist, thereby enhancing a cancer-specific immune response in the patient as compared to the cancer-specific immune response in the patient following administration of the TGF-β1 antagonist alone.

29. A method for enhancing a cancer-specific immune response in a cancer patient who has received or is receiving treatment with a CD155/TIGIT antagonist, the method comprising:
administering to the patient an effective amount of a TGF-β1 antagonist thereby enhancing a cancer-specific immune response in the patient as compared to the cancer-specific immune response in the patient following administration of the CD155/TIGIT antagonist alone.

30. A method for enhancing a cancer-specific immune response in a cancer patient, the method comprising: administering to the patient an effective amount of: (i) a CD155/TIGIT antagonist; and (ii) a TGF-β1 antagonist, thereby enhancing a cancer-specific immune response in the patient as compared to the cancer-specific immune response in the patient following administration of either the TGF-β1 antagonist or the CD155/TIGIT antagonist alone.

31. A method for enhancing a cancer-specific immune response in a cancer patient, the method comprising: administering to the patient an effective amount of:
(i) a CD155/TIGIT antagonist, wherein the CD155/TIGIT antagonist is an anti-CD155 antibody or antigen-binding fragment thereof, or an anti-TIGIT antibody or antigen-binding fragment thereof; and
(ii) a TGF-β1 antagonist, wherein the TGF-β1 antagonist comprises a fusion protein comprising an extracellular domain of the type 2 TGF-β receptor or a TGF-β1-binding fragment thereof and a human IgG1 Fc domain,
thereby enhancing a cancer-specific immune response in the patient as compared to the cancer-specific immune response in the patient following administration of either the TGF-β1 antagonist or the CD155/TIGIT antagonist alone.

32. The method of any one of claims 27 to 31, wherein cancer cells of the cancer express CD155.

33. The method of claim 32, wherein the cancer cells overexpress CD155 relative to normal cells of the same histological type.

34. The method of any one of claims 27 to 33, further comprising determining whether cancer cells of the cancer express or overexpress CD155.

35. The method of any one of claims 27 to 34, wherein the CD155/TIGIT antagonist is an anti-CD155 antibody or an antigen-binding fragment thereof.

36. The method of any one of claims 27 to 34, wherein the CD155/TIGIT antagonist is an anti-TIGIT antibody or an antigen-binding fragment thereof.

37. The method of claim 35 or 36, wherein the antibody is a humanized antibody or a fully human antibody.

38. The method of any one of claims 27 to 37, wherein the TGF-β1 antagonist comprises a small molecule, nucleic acid or polypeptide.

39. The method of claim 38, wherein the TGF-β1 antagonist is a soluble form of a TGF-β receptor protein.

40. The method of claim 39, wherein the TGF-β receptor protein is an extracellular domain of the type 2 TGF-β receptor or a TGF-β1-binding fragment thereof.

41. The method of claim 40, wherein the TGF-β receptor protein comprises the amino acid sequence depicted in SEQ ID NO: 5.

42. The method of any one of claims 39 to 41, wherein the TGF-β1 antagonist comprises a fusion protein comprising an extracellular domain of the type 2 TGF-β receptor, or a TGF-β1-binding fragment thereof, and a moiety that enhances the serum half-life of the TGF-β1 antagonist.

43. The method of claim 42, wherein the moiety comprises polyethylene glycol, an Fc portion of an antibody, or an albumin protein.

44. The method of claim 43, wherein the TGF-β1 antagonist comprises a fusion protein comprising an extracellular domain of the type 2 TGF-β receptor or a TGF-β1-binding fragment thereof and a human IgG Fc domain.

45. The method of claim 44, wherein the human IgG Fc domain is a human IgG1 Fc domain, a human IgG2 Fc domain, a human IgG3 Fc domain, or a human IgG4 Fc domain.

46. The method of claim 44 or 45, wherein the TGF-β1 antagonist comprises (a) an extracellular domain of the type 2 TGF-β receptor comprising the amino acid sequence depicted in SEQ ID NO: 5, and (b) a human IgG1 Fc domain comprising the amino acid sequence depicted in SEQ ID NO: 2.

47. The method of any one of claims 27 to 46, wherein the cancer-specific immune response is a T cell response.

48. The method of claim 47, wherein the T cell response in the patient is greater than the T cell response following administration of either the CD155/TIGIT antagonist or the TGF-β1 antagonist alone.

49. The method of claim 47 or 48, wherein the T cell response comprises the production of IFNγ by one or both of CD4⁺ T cells and CD8⁺ T cells.

50. The method of any one of claims 47 to 49, wherein the T cell response comprises the production of IL-2 by one or both of CD4⁺ T cells and CD8⁺ T cells.

51. The method of any one of claims 47 to 50, wherein the T cell response comprises proliferation of one or both of CD4⁺ T cells and CD8⁺ T cells.

52. The method of any one of claims 27 to 51, wherein treatment comprises delaying cancer progression in the patient.

53. The method of any one of claims 27 to 52, wherein treatment comprises enhancing a tumor-specific immune response in the patient.

54. A CD155/TIGIT antagonist and a TGF-β1 antagonist for use in treating cancer in a patient, wherein the treatment comprises administering to the patient an effective amount of a CD155/TIGIT antagonist and an effective amount of a TGF-β1 antagonist, thereby treating the patient.

55. A CD155/TIGIT antagonist for use in treating cancer in a patient who has received or is receiving treatment with a TGF-β1 antagonist, wherein the treatment comprises administering to the patient an effective amount of a CD155/TIGIT antagonist, thereby treating the patient.

56. A TGF-β1 antagonist for use in treating cancer in a patient who has received or is receiving treatment with a CD155/TIGIT antagonist, wherein the treatment comprises administering to the patient an effective amount of a TGF-β1 antagonist, thereby treating the patient.

57. The CD155/TIGIT antagonist and/or the TGF-β1 antagonist for use according to any one of claims 54 to 56, wherein cancer cells of the cancer express CD155.

58. The CD155/TIGIT antagonist and/or the TGF-β1 antagonist for use according to claim 57, wherein the cancer cells overexpress CD155 relative to normal cells of the same histological type.

59. The CD155/TIGIT antagonist and/or the TGF-β1 antagonist for use according to any one of claims 54 to 58, further comprising determining whether cancer cells of the cancer express or overexpress CD155.

60. The CD155/TIGIT antagonist and/or the TGF-β1 antagonist for use according to any one of claims 54 to 59, wherein the CD155/TIGIT antagonist is an anti-CD155 antibody or an antigen-binding fragment thereof.

61. The CD155/TIGIT antagonist and/or the TGF-β1 antagonist for use according to any one of claims 54 to 60, wherein the CD155/TIGIT antagonist is an anti-TIGIT antibody or an antigen-binding fragment thereof.

62. The CD155/TIGIT antagonist and/or the TGF-β1 antagonist for use according to claim 60 or 61, wherein the antibody is a humanized antibody or a fully human antibody.

63. The CD155/TIGIT antagonist and/or the TGF-β1 antagonist for use according to any one of claims 54 to 62, wherein the TGF-β1 antagonist comprises a small molecule, nucleic acid or polypeptide.

64. The CD155/TIGIT antagonist and/or the TGF-β1 antagonist for use according to claim 63, wherein the TGF-β1 antagonist is a soluble form of a TGF-β receptor protein.

65. The CD155/TIGIT antagonist and/or the TGF-β1 antagonist for use according to claim 64, wherein the TGF-β receptor protein is an extracellular domain of the type 2 TGF-β receptor or a TGF-β1-binding fragment thereof.

66. The CD155/TIGIT antagonist and/or the TGF-β1 antagonist for use according to claim 65, wherein the TGF-β receptor protein comprises the amino acid sequence depicted in SEQ ID NO: 5.

67. The CD155/TIGIT antagonist and/or the TGF-β1 antagonist for use according to any one of claims 64 to 66, wherein the TGF-β1 antagonist comprises a fusion protein comprising an extracellular domain of the type 2 TGF-β receptor, or a TGF-β1-binding fragment thereof, and a moiety that enhances the serum half-life of the TGF-β1 antagonist.

68. The CD155/TIGIT antagonist and/or the TGF-β1 antagonist for use according to claim 67, wherein the moiety comprises polyethylene glycol, an Fc portion of an antibody, or an albumin protein.

69. The CD155/TIGIT antagonist and/or the TGF-β1 antagonist for use according to claim 68, wherein the TGF-β1 antagonist comprises a fusion protein comprising an extracellular domain of the type 2 TGF-β receptor or a TGF-β1-binding fragment thereof and a human IgG Fc domain.

70. The CD155/TIGIT antagonist and/or the TGF-β1 antagonist for use according to claim 69, wherein the human IgG Fc domain is a human IgG1 Fc domain, a human IgG2 Fc domain, a human IgG3 Fc domain, or a human IgG4 Fc domain.

71. The CD155/TIGIT antagonist and/or the TGF-β1 antagonist for use according to claim 69 or 70, wherein the TGF-β1 antagonist comprises (a) an extracellular domain of the type 2 TGF-β receptor comprising the amino acid sequence depicted in SEQ ID NO: 5, and (b) a human IgG1 Fc domain comprising the amino acid sequence depicted in SEQ ID NO: 2.

72. The CD155/TIGIT antagonist and/or the TGF-β1 antagonist for use according to any one of claims 54 to 71, wherein treatment comprises a cancer-specific immune response in the patient.

73. The CD155/TIGIT antagonist and/or the TGF-β1 antagonist for use according to claim 72, wherein the cancer-specific immune response is a T cell response.

74. The CD155/TIGIT antagonist and/or the TGF-β1 antagonist for use according to claim 73, wherein the T cell response in the patient is greater than the T cell response following administration of either the CD155/TIGIT antagonist or the TGF-β1 antagonist alone.

75. The CD155/TIGIT antagonist and/or the TGF-β1 antagonist for use according to claim 73 or 74, wherein the T cell response comprises the production of IFNγ by one or both of CD4⁺ T cells and CD8⁺ T cells.

76. The CD155/TIGIT antagonist and/or the TGF-β1 antagonist for use according to any one of claims 73 to 75, wherein the T cell response comprises the production of IL-2 by one or both of CD4⁺ T cells and CD8⁺ T cells.

77. The CD155/TIGIT antagonist and/or the TGF-β1 antagonist for use according to any one of claims 73 to 76, wherein the T cell response comprises proliferation of one or both of CD4⁺ T cells and CD8⁺ T cells.

78. The CD155/TIGIT antagonist and/or the TGF-β1 antagonist for use according to any one of claims 54 to 77, wherein treatment comprises delaying cancer progression in the patient.

79. The CD155/TIGIT antagonist and/or the TGF-β1 antagonist for use according to any one of claims 54 to 78, wherein treatment comprises enhancing a tumor-specific immune response in the patient.


SEQUENCE LISTING (11 sequences)

SEQ ID NO: 1 - protein, 330 aa, Homo sapiens [sequence omitted]

SEQ ID NO: 2 - protein, 231 aa, Homo sapiens
  misc_feature: Human IgG1 Fc domain
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 592 aa, Homo sapiens
  misc_feature: Human TGF-beta-RII Isoform A Precursor Polypeptide
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 4 - protein, 567 aa, Homo sapiens
  misc_feature: Human TGF-beta-RII Isoform B Precursor Polypeptide
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 5 - protein, 137 aa, Homo sapiens
  misc_feature: Extracellular domain of Human TGF-beta-RII (Thr23-Asp159)
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 6 - protein, 425 aa, Homo sapiens
  misc_feature: Extracellular domain of Type 2 TGF-beta- receptor nucleic acid sequence
GGATCCACCATCCCCCCACACGTGCAGAAATCCGTGAACAACGACATGATCGTGACCGACAACAACGGCGCTGTGAAGTTCECCCAGCTGTGCAAGTTCTGCGAEGTGCGGTTCTCTACCTGCGACAACCAGAAATCCTGCATGTCCAACTGCTCCATCACCTCCATETGCGAGAAGCCCCAGGAAGTGTGCGTCGCCGTCTGGCGGAAGAACGACGAGAACATCACCETGGAAACCGTGTGCCACGACCCCAAGCTGCCCTACCACGACTTCATCCTGGAAGATGCCGCCTCCCCCAAGTGCATCATGAAGGAAAAGAAGAAGCCCGGCGAGACTTTCTTCATGTGCAGCTGCTCCTCCGACGAGTGCAACGACAACATCATCTTCTCCGAAGAGTACAACACCTCCAACCCEGACTGAAGCTT

SEQ ID NO: 7 - protein, 51 aa, Artificial Sequence
  Synthetic linker
  misc_feature (2)..(51): At least 1 and up to 10 Gly4Ser may be present
SGGGGSGGGGSGGGGSGGGGSGGGGSGGGGSGGGGSGGGGSGGGGSGGGGS

SEQ ID NO: 8 - protein, 16 aa, Artificial Sequence
  Synthetic linker
SGGGGSGGGGSGGGGS

SEQ ID NO: 9 - protein, 21 aa, Artificial Sequence
  Synthetic linker
SGGGGSGGGGSGGGGSGGGGS

SEQ ID NO: 10 - protein, 30 aa, Artificial Sequence
  Synthetic linker
  misc_feature (1)..(30): At least 1 and up to 6 Gly4Ser may be present
GGGGSGGGGSGGGGSGGGGSGGGGSGGGGS

SEQ ID NO: 11 - protein, 24 aa, Artificial Sequence
  Synthetic linker
  misc_feature (1)..(24): At least 1 and up to 6 Gly3Ser may be present
GGGSGGGSGGGSGGGSGGGSGGGS